SEQUENCE LISTING (82 sequences)

SEQ ID NO: 1 - DNA, 1527 bp, Zea mays
atgggggccttcgcgaaggaggagcaccagttcctcgccgagctcggcctcgcgcagcgcaacccgggcgcgttcgcatgcggtgcgtggggcggctcaggtcccaccgtcacctccacgagcccgaccaacaatcaggtcatcgcagaggtcgtggaggcgtccgtgcacgactacgaggagggcatgcgcgcctgcttcgacgccgctaagacatggatggcgattcctgctccaaagcgaggagagatcgttaggcaaatcggtgatgcactgagagcaaagctccatcaccttggcaggcttgtgtcacttgagatggggaaaattcttcctgaagggatcggtgaggttcaggaaatcattgacatgtgtgattatgctgtgggactaagtcgccaactaaatggatccattataccatctgaacgaccaaaccatatgatgatggaggtgtggaatccccttggagttgtgggtgttataacggcatttaatttcccttgtgctgtgcttggttggaatgcttgcattgctttggtctgtggaaattgtgtcgtctggaaaggtgctccaacaactccactgatcactattgcaatgacaaaaatagtggctagtgtattggagaagaacaacttaccaggcgcaatttttacatctttttgtgggggcactgaaattggtcaagcaattgcccttgacattaggatacctttggtttcattcactggaagtacaagggctggtctaatggttcagcaacaagttagtgcaagatttggtaaatgccttcttgaacttagcggaaacaatgccatcattgttatggatgatgcagatatccagctagctgtgcgatctgtcttgttcgctgctgttggtacagcaggacaacgctgcactacatgtcgtaggctgattcttcatgagaacatatatcaaaccttccttgatcagcttgttgaggtatacaaacaagtccgaattggggatccgttggagaaaggcaccttactgggaccactgcacactcctgcttcaaaagagaactttttgaaaggcattcagactatcaaatctcagggagggaaaatcctttttggaggatctgccattgaatcagaaggaaactttgtgcaaccaacaattgtggaaattacaccttctgcaccagttgtgaaagaagaactctttggtcctgtcctttatgctatgaaatttcagaccttgaaggaagcaattgaaatcaataactctgttcctcaaggattgagcagttctatatttacaaagaggccagatattatttttaagtggctcgggccccatggtagtgattgtggcatagtgaatgtgaacatacctactaatggtgctgaaattggtggagcatttggtggagaaaaagcaactggtggtggacgagaagcagggagtgattcctggaagcagtacatgaggagggcgacttgtacaatcaactatgggagcgagctacctctagcccagggaatcaattttggt

SEQ ID NO: 2 - protein, 509 aa, Zea mays
MGAFAKEEHQFLAELGLAQRNPGAFACGAWGGSGPTVTSTSPTNNQVIAEVVEASVHDYEEGMRACFDAAKTWMAIPAPKRGEIVRQIGDALRAKLHHLGRLVSLEMGKILPEGIGEVQEIIDMCDYAVGLSRQLNGSIIPSERPNHMMMEVWNPLGVVGVITAFNFPCAVLGWNACIALVCGNCVVWKGAPTTPLITIAMTKIVASVLEKNNLPGAIFTSFCGGTEIGQAIALDIRIPLVSFTGSTRAGLMVQQQVSARFGKCLLELSGNNAIIVMDDADIQLAVRSVLFAAVGTAGQRCTTCRRLILHENIYQTFLDQLVEVYKQVRIGDPLEKGTLLGPLHTPASKENFLKGIQTIKSQGGKILFGGSAIESEGNFVQPTIVEITPSAPVVKEELFGPVLYAMKFQTLKEAIEINNSVPQGLSSSIFTKRPDIIFKWLGPHGSDCGIVNVNIPTNGAEIGGAFGGEKATGGGREAGSDSWKQYMRRATCTINYGSELPLAQGINFG

SEQ ID NO: 3 - protein, 549 aa, Zea mays
MSRLLSRQHLAAVRRSAPFACVSRWLHTPSFATVSPQEVSGSSPAEVQNFVQGSWTASANWNWIVDPLNGDKFIKVAEVQGTEIKPFVESLSKCPKHGLHNPLKAPERYLMYGDISAKAAHMLGQPAVLDFFAKLIQRVSPKSYQQALAEVQVSQKFLENFCGDQVRFLARSFAVPGNHLGQRSNGYRWPYGPVAIITPFNFPLEIPLLQLMGALYMGNKPVLKVDSKVSIVMEQMIRLLHDCGLPAEDMDFINSDGAVMNKLLLEANPKMTLFTGSSRVAEKLAADLKGRVKLEDAGFDWKILGPDVQEVDYVAWVCDQDAYACSGQKCSAQSVLFMHKNWSSSGLLEKMKKLSERRKLEDLTIGPVLTVTTEAMIEHMNNLLKIRGSKVLFGGEPLANHSIPKIYGAMKPTAVFVPLEEILKSGNFELVTKEIFGPFQVVTEYSEDQLELVLEACERMNAHLTAAVVSNDPLFLQDVLGRSVNGTTYAGIRARTTGAPQNHWFGPAGDPRGAGIGTPEAIKLVWSCHREVIYDVGPVPESWALPSAT

SEQ ID NO: 4 - protein, 496 aa, Zea mays
MGRTEAADDGAESGGLGLGVGVGVGETVRELREAYESGRTRSLAWRQAQLRGLLRLLEEKEVEAFQALHKDLGKHHAEAYRDEVGVLIKSANGALQQLGKWMAPEKVRVPLIAWPATAQVVPEPLGVVLVFSCWNVPLGLSLEPLIGAIAAGNAVALKPSELSPCTARFLGDNIGRYMDSSAVKVVQGGPDVGVQLMEHRWDKVLFTGSPRIARAVMAAASRHLTPVALELGGKCPCIFDAMGSARDLQISVNRMIAGKWSSCAGQACIAIDYVLVEERFAPILIKVLKSTLKRFFPEADHMARIVNERHFERLSNLLKDRSVAPSVLHGGSMDSKNLCIEPTILLNPPLDSAIMTEEIFGPLLPIITVKNIEDSIAFVKAMPKPLAIYAFTRDAALRRRIVDETSSGSVTFNDAVVQYAIDGLPFGGVGQSGFGQYHGKYSFEMFSHKKAVMKRGYLVELTLRYPPWDESKVTLMRYLYRFNYFAFVLSFLGLRR

SEQ ID NO: 5 - protein, 485 aa, Zea mays
MGSVPEEKAKLGFGGLVGDLREVYESGRTQGLEWRQSQLRGLVRLLEEKEEEIFDVLHEDLGKHRGEAFRDEVGVLKKSVVDKLQNLKNWAAPEKAHTPLVAFPATALVVPEPLGVVLVFSCWNLPIGLALEPLSGALAAGNAVVVKPSELAPATSAFLAANIPKYLDSKAVKVVEGGPEVGEKLMEHRWDKVLFTGSSRVGRLIMAQAAKHLTPVALELGSKCPCIVDWLDSDRDSQVAVNRIIGAKWSTCSGQACIAIDYLLVEEEFAPILIEMLKSTLERFFTKPEYMARILNEKQFQRLSGFLADRRVASSVVHGGHFNPKTLSMEPTLLLNPPLDSDIMTEEIFGPLLPIITVKKIEDSIKFLRSKPKPLAIYAFTRNEKLKQRIIDETSSGSITFNDAIVQYGLDSIPFGGVGHSGFGQYHGKYSFDMFSHKKAVLKRSFLVEFMFRYPPWDETKIGMLRRVYRFDYVSLFLALIGLRR

SEQ ID NO: 6 - protein, 490 aa, Zea mays
MAEETVQELRASFASGRTRRAEWRAEQLKGLIRMIDEKEAEISAALHEDLAKPHMESYLHEISITRSSCKFALDGLKSWMKPEKVIIFSSYCHCAQIPAALTTFPSSAQIVPEPLGVVLIISAWNYPFILSIDPVIGAIAAGNAVVLKPSEIAPATSSLLAKLLPEYVDNSCIKVVEGGVAETTSLLEQRWDKIFYTGNGTVGRIVMAAAAKHLTPVALELGGKSPVVVDSNVDLHVAVKRIVVGKWGCNNGQACIAPDYIITTKSFAPELVASLKRVLERFYGEDPLQSADLSRIVNSKHFRRLTELIEEKSVADKIVYGGEVDEKQLKIAPTLLLDVPQDSAIMTGEIFGPLLPIVTVEKIEESFDLINARPKPLAAYLFTKNKKLQEEFVADVPAGGMLVNDTVLHLANPYMPFGGVGDSGMGCYHGKFGFDCFSHKKGVLVRGFGGEANARYPPYTTEKQKILRGLINGSFIALILALLGFPREKR

SEQ ID NO: 7 - protein, 478 aa, Zea mays
MAEETVRELRASFAAGQTRPAEWRAAQLKGLIRMIDEKEAEISAALHEDLAKPHMESFLHEISLTKSSCKFALKGLKNWMKPEKVPAAITTFPSSAQIVPEPLGVVLIISAWNYPFILSIDPVIGAIAAGNAVVLKPSEIAPATSSLLAKLLPEYVDNSCIKVVEGSVPETTALLEQRWDKIFYTGNGTVGRIVMAAAAKHLTPVALELGGKSPVIVDSNVDLHVAAKRIVVGKWGCNNGQACIAPDYIITTKSFAPELVASFKRVLERFYGEDPLESADLSRIVNSKQFKRLTNLIEEKRVADKIVYGGKADEKQLKISPTLLLDVPEDSEIMTGEIFGPLLPIVTVEKIEESFDLINAKPKPLAAYLFTKNRKLQEEFVASVPAGGMLVNDTALHLTNPYMPFGGVGDSGMGCYHGKFGFDCFSHKKGVLIRGFGGEANARYPPYTTEKQKILRGLINGSFIALILALLGFPREKR

SEQ ID NO: 8 - protein, 274 aa, Zea mays
MDAEAAAAAAATAVEERERLRRSFASGRTRPAAWREAQLRGLLRMATEREDDICAALHADLAKPLTECYVHEISLVISSCKFALKNLKKWMKPRKVPGGLLTFPSAASVAAEPLGVVLVISAWNYPFLLAIDPVVGAFAAGNAVALKPSEVAPATSLLLADLLPRYVDPSCVRVVQGGIPETTALLELQWDKIFYTGNSRVGRIVMSYAAKHLTPVVLGARWQMPCRRRLPTSTSTSLPRGSPPASGAATAAKPASRRTTSSLPNRSRQSCWSP

SEQ ID NO: 9 - protein, 491 aa, Zea mays
MDAEAAAAAAATAVEERERLQRSFASERTRPAAWREAHLRALLRMATEREEDICAALHADLAKPLTECYVHEISLVISSCKFALKNLKKWMKPRKVPGGLLTFPSAASVAAEPLGVVLVISAWNYPFLLAIDPVVGAFAAGNAVALKPSEVAPATSLLLADLLPRYVDPSCVRVVQGGIAETTALLELQWDKIFYTGNSRVGRIVMSYAAKHLTPVVLELGGKCPVVVDSDVNLHVAAKRIAAGKWGCNSGQACVSPDYVVTTKSFAPKLLESLKRVLFEFYGEEPLRSPDLSRVVNSNHFNRLMALMDDYSVSGNVAFGGQIDERRLKIAPTLLLDVPLDSAMMKEEIFGPLLPIITVDKIGESFAVINSMPKPLAAYLFSNDGQLKQQFERTVSAGGIMFNDTGIHLTNPNLPFGGVGESGMGAYHGAFSFDAFSHRKAVLDRSFLGEARARYPPYTPAKLAILRGVLNGSPLATVQAAAGCTGGASAD

SEQ ID NO: 10 - protein, 527 aa, Zea mays
MAMAMMAMRRAVALGARHIPAAAASSFRVVSLRHMSADASAAMEKIRAAGLLRTQGLIAGQWVDAYDGKTIEVQNPATGEVLANVSFMGSRETSDAIASAHSTFYSWSKLTASERSKALRKWYDLIISHKEELALLMTLEQGKPMKEALGEVNYGASFIEYFAEEAKRIYGDIIPPTLSDRRLLVLKQPVGVVGAITPWNFPLAMITRKVGPALACGCTVVVKPSEFTPLTALAAADLALQAGIPAGALNVVMGNAAEIGDALLQSTQVRKITFTGSTAVGKKLMAGSANTVKKVSLELGGNAPCIVFDDADIDVAVKGSLAAKFRNSGQTCVCANRILVQEGIYEKFAKAFIQAVQSLKVGNGLEESTSQGPLINEAAVQKVEKFINDATSKGANVMLGGKRHSLGMSFYEPTVVGNVSNDMLLFREEVFGPVAPLIPFKTEEEAVHMANDTNAGLAAYIFTKSIPRSWRVSESLEYGLVGVNEGIISTEVAPFGGVKQSGLGREGSKYGIDEYLELKYICMGNLG

SEQ ID NO: 11 - protein, 502 aa, Zea mays
MATANGSSKGPFEVPKVEVRFTKLFIDGKFVDAVSGKTFETRDPRTGEVIASIAEGGKADVDLAVKAAREAFDNGPWPRMTGYERGRILHRFADLIDEHVEELAALDTVDAGKLFAVGKARDIPGAAHLLRYYAGAADKVHGATLKMAQRMHGYTLKEPVGVVGHIVPWNYPTTMFFFKVGPALAAGCAVVVKPAEQTPLSALFYAHLAREAGVPAGVLNVVPGFGPTAGAAVAAHMDVDKVSFTGSTEVGRLVMRAAAESNLKPVSLELGGKSPVIVFDDADLDMAVNLVNFATYTNKGEICVAGTRIYVQEGIYDEFVKKAAELASKSVVGDPFNPSVSQGPQVDKDQYEKVLRYIDIGKREGATLVTGGKPCGDKGYYIEPTIFTDVKDDMTIAQDEIFGPVMALMKFKTVEEVIQKANNTRYGLAAGIVTKNIDVANTVSRSIRAGAIWINCYFAFDPDAPFGGYKMSGFGKDMGMDALDKYLQTKTVVTPLYNTPWL

SEQ ID NO: 12 - protein, 506 aa, Zea mays
MMASQAMVPLRQLFVDGEWRPPAQGRRLPVVNPTTEAHIGEIPAGTAEDVDAAVAAARAALKRNRGRDWARAPGAVRAKYLRAIAAKVIERKHELAKLEALDCGKPYDEAAWDMDDVAGCFEYFADQAEALDKRQNSPVSLPMETFKCHLRREPIGVVGLITPWNYPLLMATWKVAPALAAGCAAVLKPSELASVTCLELADICKEVGLPPGVLNIVTGLGPDAGAPLSAHPDVDKVAFTGSFETGKKIMAAAAPMVKPVTLELGGKSPIVVFDDVDIDKAVEWTLFGCFWTNGQICSATSRLLVHTKIAKEFNEKMVAWAKNIKVSDPLEEGCRLGPVVSEGQYEKIKKFILNAKSEGATILTGGVRPAHLEKGFFIEPTIITDITTSMEIWREEVFGPVLCVKEFSTEDEAIELANDTQYGLAGAVISGDRERCQRLSEEIDAGIIWVNCSQPCFCQAPWGGNKRSGFGRELGEGGIDNYLSVKQVTEYISDEPWGWYRSPSKL

SEQ ID NO: 13 - protein, 506 aa, Zea mays
MAPPQTIPRRGLFIGGAWREPCLGRRLPVVNPATEATIGDIPAGTAEDVEIAVAAARDAFSRDGGRHWSRAPGAVRANFLRAIAAKIKDRKSELALLETLDSGKPLDEASGDMDDVAACFEYYADLAEALDGKQQSPISLPMENFKSYVLKEPIGVVGLITPWNYPLLMATWKVAPALAAGCTTILKPSELASVSCLELGAICMEIGLPPGVLNIITGLGPEAGAPLSSHSHVDKVAFTGSTETGKRIMISAAQMVKPVSLELGGKSPLIVFDDIGDIDKAVEWTMFGIFANAGQVCSATSRLLLHEKIAKKFLDRLVAWAKNIKVSDPLEEGCRLGSVISEGQYEKIKKFISTARSEGATILYGGGRPQHLRRGFFLEPTIITDVSTSMQIWQEEVFGPVICVKEFRTESEAVELANDTHYGLAGAVISNDQERCERISKALHSGIIWINCSQPCFVQAPWGGNKRSGFGRELGEWGLDNYLTVKQVTKYCSDEPWGWYQPPSKL

SEQ ID NO: 14 - protein, 221 aa, Zea mays
MASPAMVPLRQLFVDGEWRPPAQGRRLPVVNPTTEAHIGEIPAGTAEDVDAAVAAARAALKRNRGRDWARAPGAVRAKYLRAIAAKVIERKPELAKLEALDCGKPYDEAAWDMDDVAGCFEYFADQAEALDKRQNSPVSLPMETFKCHLRREPIGVVGLITPWNYPLLMATWKIAPALAAGCTAVLKPSELASVTCLELADICERSRSPFWCLEHCDRIRS

SEQ ID NO: 15 - protein, 511 aa, Zea mays
MASNGCNGNGNGNGNGKAAPAGVVVPEIKFTKLFINGEFVDAASGKTFDTRDPRTGDVLAHVAEADKADVDLAVKSARDAFEHGKWPRMSGYERGRIMSKLADLVEQHTEELAALDGADAGKLLLLGKIIDIPAATQMLRYYAGAADKIHGDVLRVSGRYQGYTLKEPIGVVGVIIPWNFPTMMFFLKVSPALAAGCTVVVKPAEQTPLSALYYAHLAKMAGVPDGVINVVPGFGPTAGAALASHMDVDSVAFTGSTEVGRLIMESAARSNLKTVSLELGGKSPLIIFDDADVDMAVNLSRLAVFFNKGEVCVAGSRVYVQEGIYDEFVKKAVEAARSWKVGDPFDVTSNMGPQVDKDQFERVLKYIEHGKSEGATLLTGGKPAADKGYYIEPTIFVDVTEDMKIAQEEIFGPVMSLMKFKTVDEVIEKANCTRYGLAAGIVTKSLDVANRVSRSVRAGTVWVNCYFAFDPDAPFGGYKMSGFGRDQGLAAMDKYLQVKSVITALPDSPWY

SEQ ID NO: 16 - protein, 516 aa, Zea mays
MVSESNRGGADRTTAAGEERGQLLFDVPEIRFTKLFINGSFVDAVSGRTFETRDPRTGGVIASVAEADKEDVDLAVRAARAAFDHGEWPRMSGSERGRIMARLADLVEERADELAALESLDAGKHPAVTRAVDVGNAAGSLRYFAGAADKIHGETLKMPGQFQGHTLREPLGVAGVIIPWNFPSTMFAVKVAPALAAGCALVVKPAEQTPLSALYLAQLAKQAGVPDGVINVVPGFGPTAGAALASHMDVDMVSFTGSTEVGRLIMKASAESNLKPVYLELGGKSPLIVFDDADLDMAVELAVGASFFNKGEACVAASRVYVQERVYDRFEERLAERMRSWVVGDPFSDPSADQGPQVDKAQYERVLSYIDHGKREGATLLTGGRPCGPEGKGYYIEPTVFTNVKEDMIIAKEEIFGPVMCLMKFKTVEEAIARANDTRYGLGAGVVTRDLDVANRVVRSVRAGVVWVNCYFAMGSDCPFGGRKMSGFGKDEGMHALDKYLAVKSVVTPLRASPWI

SEQ ID NO: 17 - protein, 552 aa, Zea mays
MAATVRRAASSVLSRFFLTKPSPSPAASAAGNKSALLGSGAAALRHRFSTAPASAAAAAEEPIQPAVEVKHTQLLINGNFVDAASGKTFPTLDPRTGEVIARVAEGDSEDIDRAVAAARRAFDEGPWPRMTAYERCRVLLRFADLIERHAEEVAALETWDNGKTLAQAAGAEVPMVARCVRYYAGWADKIHGLVAPADGAHHVQVLHEPVGVAGQIIPWNFPLLMFAWKVGPALACGNTVVLKTAEQTPLSALYVANLLHEAGLPEGVLNVVSGFGPTAGAALCSHMGVDKLAFTGSTGTGQIVLELAARSNLKPVTLELGGKSPFIVMDDADVDQAVELAHQAVFFNQGQCCCAGSRTFVHERVYDEFVEKSKARALKRVVGDPFRDGVEQGPQIDGEQFNKILRYVQSGVDSGATLVAGGDRVGDRGFYIQPTVFADAKDEMKIAREEIFGPVQTILKFSGVEEVIRRANATPYGLAAGVFTRSLDAANTLSRALRAGTVWVNCYDVFDATIPFGGYKMSGVGREKGIYALRNYLQTKAVVTPIKNPAWL

SEQ ID NO: 18 - protein, 550 aa, Zea mays
MAATVRRAASSVLSRFLLTKPSSPASAAGNKSALRAGDGLYGLLPGALHRFSTAAAAEEPIQPAVEVKHTQLLINGNFVDAASGKTFPTLDPRTGEVIARVAEGDSEDIDRAVAAARRAFDEGPWPRMTAYERCRVLLRFADLIERHAEEVAALETWDNGKTLAQAAGAEVPMVARCVRYYAGWADKIHGLVAPADGAHHVQVLHEPVGVAGQIIPWNFPLLMFAWKVGPALACGNTVVLKTAEQTPLSALYVANLLHEAGLPEGVLNVVSGFGPTAGAALCSHMGVDKLAFTGSTGTGQIVLELAARSNLKPVTLELGGKSPFIVMDDADVDQAVELAHQAVFFNQGQCCCAGSRTFVHERVYDEFVEKSKARALKRVVGDPFRDGVEQGPQIDGEQFNKILRYVQSGVDSGATLVAGGDRVGDRGFYIQPTVFADAKDEMKIAREEIFGPVQTILKFSGVEEVIRRANATPYGLAAGVFTRSLDAANTLSRALRAGTVWVNCYDVFDATIPFGGYKMSGVGREKGIYALRNYLQTKAVVTPIKNPAWL

SEQ ID NO: 19 - protein, 498 aa, Zea mays
MALAGTGVFAEILDGEVYRYYADGEWRTSASGKSVAIVNPTTRKTQYRVQACTQEEVNKAMDAAKVAQKAWARTPLWKRADVLHKAAAILKEHKAPIAECLVKEIAKPAKDAVSEVVRSGDLVSYTAEEGVRILGEGKLVVSDSFPGNERNKYCLSSKIPLGVVLAIPPFNYPANLAGSKIGPALIAGNALVLKPPTQGAVAALHMVHCFHLAGFPKGLISCVTGKGSEIGDFLTMHPGVNCISFTGGDTGIAISKKAGMVPLQMELGGKDACIVLEDADLDLVSANIVKGGFSYSGQRCTAVKVVLIMESIADAVVQKVNAKLAKLKVGPPEDDSDITPVVTESSANFIEGLVMDAKEKGATFCQEYRREGNLIWPLLLDHVRPDMRIAWEEPFGPVLPVIRINSVEEGIHHCNASNFGLQGCIFTRDINKAILISDAMETGTVQINSAPARGPDHFSFQGLKDSGIGSQGITNSINMMTKVKSTVINLPSPSYTMG

SEQ ID NO: 20 - protein, 593 aa, Zea mays
MAFWWPLLVLAAAYALCRLLLFLIPPTVPSIDVDASDVLAKEDSFIYIPRRGKSTQTDKVQCYEPATMKYLGYFPVVTPDEVKEHVAQSRKAQRIWAKSSFKQRRQFLRILLKYILEHQDLICEVSSRDTGKTMVDASLGEIMTTCEKITWLLDEGEKWLKPEYRSTGRSMLHKRAKVEFYPLGVIGAIVSWNYPFHNVFNPVLAAVFSGNAAVIKVSEHATWSGCFYFRIIQAALSAVGAPENLVHIITGFAETGQALVSSVDKIIFVGSPGVGKMIMKRASETLIPVTLELGGKDSFIVCEDVDLPSVVQVATRAALQSSGQNCAGAERFYVHDDIYSAFVSQIVKTVKSISVGPPLSGRYDMGAICMIEHSEKLQNLVNDALDKGAEIAVRGSFGNLGEDAVDQFFPPTVLVNVDHTMKIMQEETFGPIIPIMKFSSDEEAIKLANDSKYGLGCAVFSGNQKRAIRIASQLHCGVAAINDFASSYMCQSLPFGGVKDSGFGRFAGVEGLRACCLVKSVVEDRLWPYIRTVIPKPIQYPVSEHGFEFQQLLVETLYGYSVWDRLRSLVNLIKMVTEQNSAPASNATTKKRR

SEQ ID NO: 21 - protein, 537 aa, Zea mays
MLRSALFRSAPGLRRSPATAHLSTAAAAAAAWLSNGPASAPSRVRLLIGGEFVESRADEHVDVTNPATQEVVSRIPLTTADEFKAAVDAARTAFPGWRNTPVTTRQRVMFKFQELIRANMDKLAENITTEQGKTLKDAWGDVFRGLEVVEHACGMGTLQMGEYVSNVSNGIDTFSIREPLGVCAGICPFNFPAMIPLWMFPIAVTCGNTFVLKPSEKDPGAAMMLAELAMEAGLPKGVLNIVHGTNDVVNNICDDEDIKAVSFVGSNTAGMHIYSRASAAGKRVQCNMGAKNHAIILPDADRDATLNALIAAGFGAAGQRCMALSTAVFVGGSESWEDELVKRASGLVVSSGMVNDADLGPVISRQAKDRICKLVQSGVDSGARILLDGRKIVVPRYEDGNFVGPTILADVKSDMECYKEEIFGPVLLLMKAESLEDAIQIINRNKYGNGASIFTTSGVSARKFQTDIEAGQVGINVPIPVPLPFFSFTGSKASFAGDLNFYGKAGVQFFTQIKTITQQWKESPVQRVSLSMPTSQK

SEQ ID NO: 22 - protein, 549 aa, Zea mays
MARRAASSLVSRCLLARAPAGAPPAAPSAPRRTVPADGMHRLLPGVLQRFSTAAAVEEPITPSVHVNYTKLLINGNFVDSASGKTFPTLDPRTGEVIAHVAEGDAEDINRAVAAARKAFDEGPWPKMTAYERSRILLRFADLIEKHNDELAALETWDNGKPYEQAAQIEVPMVARLMRYYAGWADKIHGLIVPADGPHHVQILHEPIGVAGQIIPWNFPLLMYAWKVGPALACGNTLVLKTAEQTPLSALYISKLLHEAGLPEGVVNVVSGFGPTAGAALASHMDVDKIAFTGSTDTGKIILELAAKSNLKTVTLELGGKSPFIIMDDADVDHAVELAHFALFFNQGQCCCAGSRTFVHERVYDEFVEKAKARALKRVVGDPFRKGVEQGPQIDDEQFNKILRYIRYGVDGGATLVTGGDRLGDKGFYIQPTIFSDVQDGMKIAQEEIFGPVQSILKFKDLNEVIKRANASQYGLAAGVFTNSLDTANTLTRALRAGTVWVNCFDVFDAAIPFGGYKMSGIGREKGVDSLKNYLQVKAVVTPIKNAAWL

SEQ ID NO: 23 - protein, 716 aa, Zea mays
MATADQSRIFMKDVKRVIIKVGTAVVTRHDGRVSTGNFGVLCEQVKEINALGYEVIIVSSGAVGVGKQRLNYRKLVNSSFADMQKPQTELDGKACAAVGQSGLMALYDMLFTQLDVSSSQLLVTDSDFENPNFRERLRETVESLLDLKVVPIFNENDAISTRKAPYEDSSGIFWDNDSLAGLLAIELKADLLVLLSDVDGLYSGPPSEPGSKIIHTYIKDKHYSGITFGDKSRVGRGGMTAKVKAAFVASNSGTPVVITSGFASQSIVRVLQGEKIGTLFHKDASLWEPSKDVSAREMAVAARECSRRLQNLSSDERKKILLDIADALEQNEDLIRTENEADVSAAQDAGYQKSLVDRLTLKPEKIASLAKSIRTLANMEDPINQILKRTEVAEDLVLEKTSCPLGVLLIVFESRPDALVQIASLAVRSGNGLLLKGGKEAMRSNTVLHKVITGAIPDNVGQKLIGLVTSRDEIADLLKLDDVIDLVIPRGSNKLVSQIKASTKIPVLGHADGICHVYIDKSADMNMAKRIVMDAKTDYPAACNAMETLLVHKDLIKAPGLDDILLSLKTEGVAIYGGPVAHEVLCIPKADSFHHEYSSMACTIEFVDDVQSAINHIHRYGSAHTDCIITTDDKVAETFLRQVDSAAVFHNASTRFSDGARFGLGAEVGISTGRIHARGPVGVEGLLTTRWIMRGSGQVVNGDKNVAYTHKNLPLQ

SEQ ID NO: 24 - protein, 731 aa, Zea mays
MGRGGIGGAAAMAMAMETADPARAFVKDVKRIIIKVGTAVVTGMNGRLAMGRLGSLCEQVKQLNFQGYEVILVTSGAVGGGRQRLQYRKLIHSSFADLQNPQMNFDGKACAAVGQSVLMAIYDTLFSQLDVTSSQLLVTDRDFKDPSFGDQLRETVFSLLDLKVVPLFNENDAISTRRQPYEDSSGIFWDNDSLAALLAAELNADLLIMLSDVEGLYSGPPSDPQSKIIHTYVNEKHGKLISFGEKSSVGRGGMQAKVSAAANAASKGVPVVIASGFATDSIITVLKGEKIGTLFHNEANLWACSKEATAREMAVAARDCSRRLQKLSSEERKQILLDIADALEANEDAIRSENDADVEAAQVAGYEKSLVARMTLKPGKITNLARSIRKTADMEDPISHTLKRTEVAKDLVFEKAYCPLGVLLIIFESRPDALVQIASLAIRSGNGLLLKGGKEVMRSNAILHKVITGVIPDTVGKKLIGLVTSKEEIADLLALDDVIDLVIPRGSKSLVSQIKATTKIPVLGHADGICHVYIDKSADMDMAKRIVLDAKIDYPAACNAMETLLVHKDLNKSEGLDDLLVELEKEGVVIYGGPVAHDKLKVPKVDSFRHEYSSMACTVEFVDDVQSAIDHINRYGSAHTDCIITTDRSAAEAFLQQVDSAAVFHNASTRFCDGTRFGLGAEVGISTERIHARGPVGVDGLLTTRCILRGSGQVVNGDKGVVYTHKDLPLQ

SEQ ID NO: 25 - protein, 556 aa, Arabidopsis thaliana
MYRVFASRALRAKSLCDKSSTSLASLTLSRLNHSIPFATVDAEELSGAHPAEVQSFVQGKWIGSSNHNTLLDPLNGEPFIKVAEVDESGTQPFVDSLSQCPKHGLHNPFKSPERYLLYGDISTKAAHMLALPKVADFFARLIQRVAPKSYQQAAGEVFVTRKFLENFCGDQVRFLARSFAIPGNHLGQQSHGYRWPYGPVTIVTPFNFPLEIPLLQLMGALYMGNKPLLKVDSKVSIVMEQMMRLLHYCGLPAEDVDFINSDGKTMNKILLEANPRMTLFTGSSRVAEKLALDLKGRIRLEDAGFDWKVLGPDVQEVDYVAWQCDQDAYACSGQKCSAQSMLFVHENWSKTPLVSKLKELAERRKLEDLTIGPVLTFTTEAMLEHMENLLQIPGSKLLFGGKELKNHSIPSIYGALEPTAVYVPIEEILKDNKTYELVTKEIFGPFQIVTEYKKDQLPLVLEALERMHAHLTAAVVSNDPIFLQEVIGNSVNGTTYAGLRGRTTGAPQNHWFGPAGDPRGAGIGTPEAIKLVWSCHREVIYDYGPVPQGWELPPST

SEQ ID NO: 26 - protein, 484 aa, Arabidopsis thaliana
MEAMKETVEESLREMRETFASGRTRSLKWRKAQIGAIYEMVKDNEDKICNALFQDLGKHSTEAFRDELGVVLRTATVAINCLDKWAVPKHSKLPLLFYPAKGKVISEPYGTVLVLSSWNFPISLSLDPLIGAIAAGNTVLLKSSELSPNASAFLAKTIPAYLDTKAIKVIEGGPDVATILLQHQWDKIFFTGSPKIGRIIMAAAAQHLTPVTLELGGKCPTIVDHHTISKNIKSVVKRIAGGKWGSCNGQACISVDYVLIEKSFAPTLIDMLKPTIKSFFGENPKESGCLSRIANKHHVQRLSRLLSDPRVQASIVYGGSIDEDKLYVEPTILLDPPLDSEIMNEEIFGPILPIITVRDIQESIGIINTKPKPLAIYAFTNDENLKTRILSETSSGSVTFNDVMIQYMCDALPFGGVGESGIGRYHGKYSFDCFSHEKAIMEGSLGMDLEARYPPWNNFKLTFIRLAFREAYFKLILLMLGLKR

SEQ ID NO: 27 - protein, 550 aa, Arabidopsis thaliana
MTKLLEINHIQTLCFAKGFSPARLNVATSPFRISRRGGGGYCSNACIPYRLKFTCYATLSAVVKEQASDFSGKEAALLVDELRSNFNSGRTKSYEWRISQLQNIARMIDEKEKCITEALYQDLSKPELEAFLAEISNTKSSCMLAIKELKNWMAPETVKTSVTTFPSSAQIVSEPLGVVLVISAWNFPFLLSVEPVIGAIAAGNAVVLKPSEIAPAASSLLAKLFSEYLDNTTIRVIEGGVPETTALLDQKWDKIFFTGGARVARIIMAAAARNLTPVVLELGGKCPALVDSDVNLQVAARRIIAGKWACNSGQACIGVDYVITTKDFASKLIDALKTELETFFGQNALESKDLSRIVNSFHFKRLESMLKENGVANKIVHGGRITEDKLKISPTILLDVPEASSMMQEEIFGPLLPIITVQKIEDGFQVIRSKPKPLAAYLFTNNKELEKQFVQDVSAGGITINDTVLHVTVKDLPFGGVGESGIGAYHGKFSYETFSHKKGVLYRSFSGDADLRYPPYTPKKKMVLKALLSSNIFAAILAFFGFSKDS

SEQ ID NO: 28 - protein, 484 aa, Arabidopsis thaliana
MAAKKVFGSAEASNLVTELRRSFDDGVTRGYEWRVTQLKKLMIICDNHEPEIVAALRDDLGKPELESSVYEVSLLRNSIKLALKQLKNWMAPEKAKTSLTTFPASAEIVSEPLGVVLVISAWNYPFLLSIDPVIGAISAGNAVVLKPSELAPASSALLTKLLEQYLDPSAVRVVEGAVTETSALLEQKWDKIFYTGSSKIGRVIMAAAAKHLTPVVLELGGKSPVVVDSDTDLKVTVRRIIVGKWGCNNGQACVSPDYILTTKEYAPKLIDAMKLELEKFYGKNPIESKDMSRIVNSNHFDRLSKLLDEKEVSDKIVYGGEKDRENLKIAPTILLDVPLDSLIMSEEIFGPLLPILTLNNLEESFDVIRSRPKPLAAYLFTHNKKLKERFAATVSAGGIVVNDIAVHLALHTLPFGGVGESGMGAYHGKFSFDAFSHKKAVLYRSLFGDSAVRYPPYSRGKLRLLKALVDSNIFDLFKVLLGLA

SEQ ID NO: 29 - protein, 554 aa, Arabidopsis thaliana
MPFWWPLIVLAFAYAICKFLLMLIPPNVPSIDVDASDVLAHGKDTEENSFIYIPPRGRSQQSDKKVQCYEPATMKYLGYFPALSPTEVEERVTLSRKAQKTWAQSSFKLRRQFLRILLKYIIEHQELICEVSSRDTGKTMVDASLGEIMTTCEKITWLLSEGERWLKPESRSSGRAMLHKVSRVEFHPLGVIGAIVPWNYPFHNIFNPMLAAVFSGNGIVIKVSEHASWSGCFYFRIIQAALAAVGAPENLVDVITGFAETGEALVSSVDKMIFVGSTAVGKMIMRNAAETLTPVTLELGGKDAFIICEDADVSHVAQVAVRGTLQSSGQNCAGAERFYVHKDIYTAFIGQVTKIVKSVSAGPPLTGRYDMGAICLQEHSEHLQSLVNDALDKGAEIAVRGSFGHLGEDAVDQYFPPTVLINVNHNMKIMKEEAFGPIMPIMQFSTDEEVIKLANDSRYALGCAVFSGSKHRAKQIASQIQCGVAAINDFASNYMCQSLPFGGVKDSGFGRFAGIEGLRACCLVKSVVEDRFWPLIKTKIPKPIQAISGSRKRI

SEQ ID NO: 30 - protein, 496 aa, Arabidopsis thaliana
MAGTGLFAEILDGEVYKYYADGEWKTSSSGKSVAIMNPATRKTQYKVQACTQEEVNAVMELAKSAQKSWAKTPLWKRAELLHKAAAILKDNKAPMAESLVKEIAKPAKDSVTEVVRSGDLISYCAEEGVRILGEGKFLLSDSFPGNDRTKYCLTSKIPLGVVLAIPPFNYPVNLAVSKIAPALIAGNSLVLKPPTQGAVSCLHMVHCFHLAGFPKGLISCITGKGSEIGDFLTMHPAVNCISFTGGDTGISISKKAGMIPLQMELGGKDACIVLDDADLDLVASNIIKGGFSYSGQRCTAVKVVLVMESVADELVEKVKAKVAKLTVGPPEENSDITAVVSESSANFIEGLVMDAKEKGATFCQEYKREGNLIWPLLLDNVRPDMRIAWEEPFGPVVPVLRINSVEEGINHCNASNFGLQGCVFTKDINKAILISDAMETGTVQINSAPARGPDHFPFQGLKDSGIGSQGVTNSINLMTKVKTTVINLPTPSYSMG

SEQ ID NO: 31 - protein, 607 aa, Arabidopsis thaliana
MVRVKQKNLESYRSNGTYPPTWRNPTTSFAPDQHRVSIHSSLKSKTKRRRLYKEADDNTKLRSSSSTTTTTTTMLLRISGNNLRPLRPQFLALRSSWLSTSPEQSTQPQMPPRVPNLIGGSFVESQSSSFIDVINPATQEVVSKVPLTTNEEFKAAVSAAKQAFPLWRNTPITTRQRVMLKFQELIRKNMDKLAMNITTEQGKTLKDSHGDIFRGLEVVEHACGMATLQMGEYLPNVSNGVDTYSIREPLGVCAGICPFNFPAMIPLWMFPVAVTCGNTFILKPSEKDPGASVILAELAMEAGLPDGVLNIVHGTNDTVNAICDDEDIRAVSFVGSNTAGMHIYARAAAKGKRIQSNMGAKNHGLVLPDANIDATLNALLAAGFGAAGQRCMALSTVVFVGDAKSWEDKLVERAKALKVTCGSEPDADLGPVISKQAKERICRLIQSGVDDGAKLLLDGRDIVVPGYEKGNFIGPTILSGVTPDMECYKEEIFGPVLVCMQANSFDEAISIINKNKYGNGAAIFTSSGAAARKFQMDIEAGQIGINVPIPVPLPFFSFTGNKASFAGDLNFYGKAGVDFFTQIKTVTQQWKDIPTSVSLAMPTSQKQ

SEQ ID NO: 32 - protein, 508 aa, Arabidopsis thaliana
MGSANNEYEFLSEIGLTSHNLGSYVAGKWQANGPLVSTLNPANNQPIAQVVEASLEDYEQGLKACEEAAKIWMQVTAPKRGDIVRQIGDALRSKLDYLGRLLSLEMGKILAEGIGEVQEVIDMCDFAVGLSRQLNGSVIPSERPNHMMLEMWNPLGIVGVITAFNFPCAVLGWNACIALVCGNCVVWKGAPTTPLITIAMTKLVAEVLEKNNLPGAIFTAMCGGAEIGEAIAKDTRIPLVSFTGSSRVGSMVQQTVNARSGKTLLELSGNNAIIVMDDADIQLAARSVLFAAVGTAGQRCTTCRRLLLHESVYDKVLEQLLTSYKQVKIGNPLEKGTLLGPLHTPESKKNFEKGIEVIKSQGGKILTGGKAVEGEGNFVEPTIIEISADAAVVKEELFAPVLYVLKFKSFGEAVAINNSVPQGLSSSIFTRNPENIFRWIGPLGSDCGIVNVNIPTNGAEIGGAFGGEKATGGGREAGSDSWKQYMRRSTCTINYGNELPLAQGINFG

SEQ ID NO: 33 - protein, 528 aa, Arabidopsis thaliana
MVIGAAARVAIGGCRKLISSHTSLLLVSSQCRQMSMDAQSVSEKLRSSGLLRTQGLIGGKWLDSYDNKTIKVNNPATGEIIADVACMGTKETNDAIASSYEAFTSWSRLTAGERSKVLRRWYDLLIAHKEELGQLITLEQGKPLKEAIGEVAYGASFIEYYAEEAKRVYGDIIPPNLSDRRLLVLKQPVGVVGAITPWNFPLAMITRKVGPALASGCTVVVKPSELTPLTALAAAELALQAGVPPGALNVVMGNAPEIGDALLTSPQVRKITFTGSTAVGKKLMAAAAPTVKKVSLELGGNAPSIVFDDADLDVAVKGTLAAKFRNSGQTCVCANRVLVQDGIYDKFAEAFSEAVQKLEVGDGFRDGTTQGPLINDAAVQKVETFVQDAVSKGAKIIIGGKRHSLGMTFYEPTVIRDVSDNMIMSKEEIFGPVAPLIRFKTEEDAIRIANDTIAGLAAYIFTNSVQRSWRVFEALEYGLVGVNEGLISTEVAPFGGVKQSGLGREGSKYGMDEYLEIKYVCLGDMNRH

SEQ ID NO: 34 - protein, 501 aa, Arabidopsis thaliana
MENGKCNGATTVKLPEIKFTKLFINGQFIDAASGKTFETIDPRNGEVIATIAEGDKEDVDLAVNAARYAFDHGPWPRMTGFERAKLINKFADLIEENIEELAKLDAVDGGKLFQLGKYADIPATAGHFRYNAGAADKIHGETLKMTRQSLFGYTLKEPIGVVGNIIPWNFPSIMFATKVAPAMAAGCTMVVKPAEQTSLSALFYAHLSKEAGIPDGVLNIVTGFGSTAGAAIASHMDVDKVSFTGSTDVGRKIMQAAAASNLKKVSLELGGKSPLLIFNDADIDKAADLALLGCFYNKGEICVASSRVFVQEGIYDKVVEKLVEKAKDWTVGDPFDSTARQGPQVDKRQFEKILSYIEHGKNEGATLLTGGKAIGDKGYFIQPTIFADVTEDMKIYQDEIFGPVMSLMKFKTVEEGIKCANNTKYGLAAGILSQDIDLINTVSRSIKAGIIWVNCYFGFDLDCPYGGYKMSGNCRESGMDALDNYLQTKSVVMPLHNSPWM

SEQ ID NO: 35 - protein, 538 aa, Arabidopsis thaliana
MAARRVSSLLSRSFSASSPLLFRSQGRNCYNGGILRRFGTSSAAAEEIINPSVQVSHTQLLINGNFVDSASGKTFPTLDPRTGEVIAHVAEGDAEDINRAVKAARTAFDEGPWPKMSAYERSRVLLRFADLVEKHSEELASLETWDNGKPYQQSLTAEIPMFARLFRYYAGWADKIHGLTIPADGNYQVHTLHEPIGVAGQIIPWNFPLLMFAWKVGPALACGNTIVLKTAEQTPLTAFYAGKLFLEAGLPPGVLNIVSGFGATAGAALASHMDVDKLAFTGSTDTGKVILGLAANSNLKPVTLELGGKSPFIVFEDADIDKAVELAHFALFFNQGQCCCAGSRTFVHEKVYDEFVEKSKARALKRVVGDPFRKGIEQGPQIDLKQFEKVMKYIKSGIESNATLECGGDQIGDKGYFIQPTVFSNVKDDMLIAQDEIFGPVQSILKFSDVDEVIKRANETKYGLAAGVFTKNLDTANRVSRALKAGTVWVNCFDVFDAAIPFGGYKMSGNGREKGIYSLNNYLQIKAVVTALNKPAWI

SEQ ID NO: 36 - protein, 534 aa, Arabidopsis thaliana
MASRRVSSLLSRSFMSSSRSIFSLRGMNRGAQRYSNLAAAVENTITPPVKVEHTQLLIGGRFVDAVSGKTFPTLDPRNGEVIAQVSEGDAEDVNRAVAAARKAFDEGPWPKMTAYERSKILFRFADLIEKHNDEIAALETWDNGKPYEQSAQIEVPMLARVFRYYAGWADKIHGMTMPGDGPHHVQTLHEPIGVAGQIIPWNFPLLMLSWKLGPALACGNTVVLKTAEQTPLSALLVGKLLHEAGLPDGVVNIVSGFGATAGAAIASHMDVDKVAFTGSTDVGKIILELASKSNLKAVTLELGGKSPFIVCEDADVDQAVELAHFALFFNQGQCCCAGSRTFVHERVYDEFVEKAKARALKRNVGDPFKSGIEQGPQVDSEQFNKILKYIKHGVEAGATLQAGGDRLGSKGYYIQPTVFSDVKDDMLIATDEIFGPVQTILKFKDLDEVIARANNSRYGLAAGVFTQNLDTAHRLMRALRVGTVWINCFDVLDASIPFGGYKMSGIGREKGIYSLNNYLQVKAVVTSLKNPAWL

SEQ ID NO: 37 - protein, 501 aa, Arabidopsis thaliana
MAIPMPTRQLFIDGEWREPILKKRIPIVNPATEEVIGDIPAATTEDVDVAVNAARRALSRNKGKDWAKAPGAVRAKYLRAIAAKVNERKTDLAKLEALDCGKPLDEAVWDMDDVAGCFEFYADLAEGLDAKQKAPVSLPMESFKSYVLKQPLGVVGLITPWNYPLLMAVWKVAPSLAAGCTAILKPSELASVTCLELADICREVGLPPGVLNVLTGFGSEAGAPLASHPGVDKIAFTGSFATGSKVMTAAAQLVKPVSMELGGKSPLIVFDDVDLDKAAEWALFGCFWTNGQICSATSRLLVHESIASEFIEKLVKWSKNIKISDPMEEGCRLGPVVSKGQYEKILKFISTAKSEGATILHGGSRPEHLEKGFFIEPTIITDVTTSMQIWREEVFGPVLCVKTFASEDEAIELANDSHYGLGAAVISNDTERCDRISEAFEAGIVWINCSQPCFTQAPWGGVKRSGFGRELGEWGLDNYLSVKQVTLYTSNDPWGWYKSPN

SEQ ID NO: 38 - protein, 503 aa, Arabidopsis thaliana
MAITVPRRQLFIGGQWTEPVLRKTLPVVNPATEDIIGYIPAATSEDVELAVEAARKAFTRNNGKDWARATGAVRAKYLRAIAAKVIERKSELANLEAIDCGKPLDEAAWDMDDVAGCFEYYADLAEGLDAKQKTPLSLPMDTFKGYILKEPIGVVGMITPWNYPLLMAVWKVAPSLAAGCTAILKPSELASLTCLELADICREVGLPPGVLNILTGLGTEAGAPLASHPHVDKIVFTGSTTTGSSIMTSAAKLVKPVSLELGGKSPIIVFDDVDIDKAVEWTMFGCFWTNGQICSATSRLLVHERIADEFLDKLVKWTKNIKISDPFEEGCRLGPVVSKGQYERVLKFVSNARNEGATVLCGGVRPEHLKKGYFVEPAIVSNVTTSMEIWREEVFGPALCVKTFSTEDEAIQLANDSQYGLAGAVLSNDLERCDRVSKAFQAGIVWVNCSQPCFCQAPWGGTKRSGFGRELGEWGLENYLSVKQVTQYISDEPWGWYKPPSKL

SEQ ID NO: 39 - protein, 487 aa, Oryza sativa
MEEKPQHGSLGGLVAGVREEYESGRTKELEWRKAQLGGLIRMITEEEDAIFDALHDDLGKHRVESFRDEVGVLAKSVRNTLQNLKKWASPEKVDVPLISFPCNARVVPEPIGVVLIFSCWNLPIGLALEPLSGAIAAGNAVVLKPSEFAPSTAAFLAANIPKYLDANAVKVVQGGAEVGEELMEHRWDKVLFTGNARVGRIIMTKAAKHLTPVALELGSKCPCIVDCLDSKRECQVAVNRIIGAKWSTCAGQACVAIDYILVEEQFAPFLIELLKSTLKRFFTEPEYMARILNEKHFHRLTNLLEDDQVKSSIVHGGNADPKTLWIEPTIVLNPPFDSDIMMEEIFGPLLPIITVKKTEDCIAFLKSKPKPLAIYAFTNNEKLKQRIVAETSSGSVLFNDAIVQYGLDSVPFGGIGESGFGQYHGKYTFELFSHRKAVVRRSLLVEFMFRYPPWDEYKMGMLRRVFRFDYVSLVLALLAFWLLGIRR

SEQ ID NO: 40 - protein, 361 aa, Oryza sativa
MFTGCGLFACVRRCDGGDVRKRGEAGAMSSRVAADPAGVEEEGSCKNVAAASARQLAWADVESVTGGFSSRVIGHGGFSTVYLASLSSSRLGAVKVHCSSERLHRAFRQELEVLLSLRHPHIVRLLGYCDERDEGVLVFEYAPNGDLHERLHCSEVAGGVASVLPWARRVAIAFQVAMALEYLHESRHPAVIHGDIKASNVLLDANMNAKLCDFGFAHVGFSATVGCRPSARAVMGSPGYVDPHLIRSGVATKKSDVYSFGVLLLELVTGKEAVCRDTGRRLTAAVGPMLSEGKVADVVDRRLGGEHDGAEAAVMAELAMQCIGDSPGLRPSMADVVRALQEKTSALASAVGSRLDRKMMF

SEQ ID NO: 41 - protein, 597 aa, Oryza sativa
MALWWPLLVLAAAYALCRILLFLIPPTVPSIDVDASDVLEDANQNKEDSYIYIPPRKGKGAQTDKVQCYEPATMKYLGYFPALTPDEVKEHVAQARKAQKIWAKSSFKQRRQFLRILLKYILEHQDLICEISSRDTGKTMVDASLGEIMTTCEKITWLLDEGEKWLKPEYRSCGRSMLHKKAKVEFYPLGVIGAIVSWNYPFHNVFNPMLAAIFSGNAAVIKVSEHASWSGCFYFRIIQAALAAVGAPDNLVHIITGFAETGQALVSSVDKIIFVGSPGVGRMIMNRASDTLIPVTLELGGKDAFIVCEDVDLPSVVQVAVRAALQSSGQNCAGAERFYVHKDIYSTFVSQVVKIIKSISVGPPLSGRYDMGAICMIEHSEKLQNLVNDAVDKGAEIAGRGSFGHLGEDAVDQFFPPTVLVNVNHTMKIMQEEAFGPILPIMKFNSDEEVVKLANDSKYGLGCAVFSGNQKRAIKIASQLHCGVAAINDFASSYMCQSLPFGGVKDSGFGRFAGVEGLRACCLVKAVVEDRWWPYVKTMIPKPIQYPVSENGFEFQELLVETLYGLSVWDRLRSLVNLLKMISEQNNSPANTRKKSR

SEQ ID NO: 42 - protein, 499 aa, Oryza sativa
MAAVAGTGVFAEILEGEVYRYYADGEWRVSASGKSVAIVNPTTRLTQYRVQACTQEEVNKVMETAKVAQKAWARTPLWKRAELLHKAAAILKEHKTPIAECLVKEIAKPAKDAISEVVRSGDLVSYTAEEGVRILGEGKLLVSDSFPGNERNKYCLSSKVPLGVVLAIPPFNYPVNLAVSKIGPALIAGNALVLKPPTQGAVAALHMVHCFHLAGFPKGLINCVTGKGSEIGDFLTMHPGVNCISFTGGDTGIAISKKAGMVPLQMELGGKDACVVLEDADLDLVAANIVKGGFSYSGQRCTAVKVVLIMESVADIVVEKVKAKLAKLTVGPPEADSDITPVVTESSANFIEGLVMDAKEKGATFCQEYRREGNLIWPLLLDHVRPDMRIAWEEPFGPVLPVIRINSVEEGIHHCNASNFGLQGCVFTKDINKAIMISDAMETGTVQINSAPARGPDHFPFQGLKDSGIGSQGITNSINMMTKVKSTVINLPSPSYTMG

SEQ ID NO: 43 - protein, 534 aa, Oryza sativa
MLRAALLRSGSGLRRPPMAAPLSTAAAASWLSDSASSPPRVRLLIGGEFVESRADEHVDVTNPATQEVVSRIPLTTADEFRAAVDAARTAFPGWRNTPVTTRQRIMLKYQELIRANMDKLAENITTEQGKTLKDAWGDVFRGLEVVEHACGMGTLQMGEYVSNVSNGIDTFSIREPLGVCAGICPFNFPAMIPLWMFPIAVTCGNTFVLKPSEKDPGAAMMLAELAMEAGLPKGVLNIVHGTHDVVNNICDDEDIKAVSFVGSNIAGMHIYSRASAKGKRVQSNMGAKNHAIILPDADRDATLNALIAAGFGAAGQRCMALSTAVFVGGSEPWEDELVKRASSLVVNSGMASDADLGPVISKQAKERICKLIQSGADNGARVLLDGRDIVVPNFENGNFVGPTLLADVKSEMECYKEEIFGPVLLLMKAESLDDAIQIVNRNKYGNGASIFTTSGVSARKFQTDIEAGQVGINVPIPVPLPFFSFTGSKASFAGDLNFYGKAGVQFFTQIKTVTQQWKESPAQRVSLSMPTSQK

SEQ ID NO: 44 - protein, 509 aa, Oryza sativa
MGSFARKEHQFLAELGLAPRNPGSFACGAWGGSGPVVTSTNPTNNQVIAEVVEASAREYEEGMRACYDAAKTWMAIPAPKRGEIVRQIGDALRAKLHHLGRLVSLEMGKILPEGIGEVQEIIDMCDYAVGLSRQLNGSIIPSERPNHMMMEVWNPLGVVGVITAFNFPCAVLGWNACIALVCGNCVVWKGAPTTPLITIAMTKIVASVLERNNLPGSIFTAFCGGADIGQAISLDTRIPLVSFTGSTKVGLMVQQQVNARFGKCLLELSGNNAIIVMDDADIQLAVRSVLFAAVGTAGQRCTTCRRLLLHESIYRTFLDQLVEVYKQVRIGDPLENGTLLGPLHTPASRDAFLKGIQTIRSQGGKILYGGSAIESEGNFVQPTIVEISPSAPVVREELFGPVLYVMKVQNLKEAVEINNSVPQGLSSSIFTKRPDIIFKWIGPHGSDCGIVNVNIPTNGAEIGGAFGGEKATGGGREAGSDSWKQYMRRATCTINYGSELPLAQGINFG

SEQ ID NO: 45 - protein, 502 aa, Oryza sativa
MAAANGGDSKGFEVPKLEIKFTKLFINGRFVDAVSGKTFETRDPRTGEVIAKIAEGDKADIDLAVKAAREAFDHGPWPRMSGFARGRILHKFADLVEQHVEELAALDTVDAGKLFAMGKLVDIPGGANLLRYYAGAADKVHGETLKMARPCHGYTLKEPVGVVGHIVPWNYPTTMFFFKASPALAAGCTMVVKPAEQTPLSALFYAHLAKLAGVPDGVLNVVPGFGPTAGAAISSHMDIDKVSFTGSTEVGRLVMEAAAKSNLKPVSLELGGKSPVIVFDDADLDTAVNLVHMASYTNKGEICVAGSRIYVQEGIYDAFVKKATEMAKKSVVGDPFNPRVHQGPQIDKEQYEKILKYIDIGKREGATLVTGGKPCGENGYYIEPTIFTDVKEEMSIAQEEIFGPVMALMKFKTVEEAIQKANSTRYGLAAGIVTKNIDVANTVSRSIRAGAIWINCYLGFDPDVPFGGYKMSGFGKDMGMDALEKYLHTKAVVTPLYNTPWL

SEQ ID NO: 46 - protein, 507 aa, Oryza sativa
MGSTGDCGNGKAAAGGGGLVVPEIKFTKLFINGEFVDAASGKTFKTRDPRTGDVLAHIAEADKADVDLAVKAAREAFEHGKWPRMSGYERSRVMNKLADLVEQHADELAALDGADAGKLLTLGKIIDMPAAAQMMRYYAGAADKIHGESLRVAGKYQGYTLREPIGVVGVIIPWNFPTMMFFLKVSPALAAGCTIVVKPAEQTPLSALYYAHLAKLAGVPDGVINVVPGFGPTAGAALSSHMDVDSVAFTGSAEIGRAIMESAARSNLKNVSLELGGKSPMIVFDDADVDMAVSLSSLAVFFNKGEICVAGSRVYVQEGIYDEFVKKAVEAAKNWKVGDPFDAATNMGPQVDKVQFERVLKYIEIGKNEGATLLTGGKPTGDKGYYIEPTIFVDVKEEMTIAQEEIFGPVMSLMKFKTVEEAIEKANCTKYGLAAGIVTKNLNIANMVSRSVRAGTVWVNCYFAFDPDAPFGGYKMSGFGRDQGMVAMDKYLQVKTVITAVPDSPWY

SEQ ID NO: 47 - protein, 583 aa, Oryza sativa
MAARRAASSLLSRGLIARPSAASSTGDSAILGAGSARGFLPGSLHRFSAAPAAAATAAATEEPIQPPVDVKYTKLLINGNFVDAASGKTFATVDPRTGDVIARVAEGDAEDVNRAVAAARRAFDEGPWPRMTAYERCRVLLRFADLIEQHADEIAALETWDGGKTLEQTTGTEVPMVARYMRYYGGWADKIHGLVVPADGPHHVQVLHEPIGVAGQIIPWNFPLLMFAWKVGPALACGNAVVLKTAEQTPLSALFVASLLHEAGLPDGVLNVVSGFGPTAGAALSSHMGVDKLAFTGSTGTGKIVLELAARSNLKPVTLELGGKSPFIVMDDADVDQAVELAHRALFFNQGQCCCAGSRTFVHERVYDEFVEKARARALQRVVGDPFRTGVEQGPQIDGEQFKKILQYVKSGVDSGATLVAGGDRAGSRGFYIQPTVFADVEDEMKIAQEEIFGPVQSILKFRFATLEFPRIPSDRDRLSTGIYTNTLRAMCSTVEEVVRRANATPYGLAAGVFTQRLDAANTLARALRVGTVWVNTYDVFDAAVPFGGYKMSGVGREKGVYSLRNYLQTKAVVTPIKDAAWL

SEQ ID NO: 48 - protein, 549 aa, Oryza sativa
MAAAAARRGSSLLSRCLLSRPAAAASPAVPSALRRADGTQGLLPGILQRFSTAAVAEEPISPPVQVNYTQLLIDGKFVDSASGKTFPTLDPRTGELIAHVAEGDAEDINRAVHAARKAFDEGPWPKMTAYERSRILLRFADLIEKHNDEIAALETWDNGKPYAQAANIEVPMVARLMRYYAGWADKIHGLVVPADGPHHVQVLHEPIGVAGQIIPWNFPLLMFAWKVGPALACGNTVVLKTAEQTPLSALFASKLLHEAGLPDGVVNVVSGFGPTAGAALASHMDVDKIAFTGSTDTGKVVLELAARSNLKSVTLELGGKSPFIIMDDADVDHAVELAHFALFFNQGQCCCAGSRTFVHERIYDEFVEKAKARALKRVVGDPFKNGVEQGPQIDDEQFNKILRYIKYGVDSGANLVTGGDRLGDKGYYIQPTIFSDVQDNMRIAQEEIFGPVQSILKFNDLNEVIKRANASQYGLAAGVFTNNLNTANTLTRALRVGTVWVNCFDVFDAAIPFGGYKQSGIGREKGIDSLKNYLQVKAVVTPIKNAAWL

SEQ ID NO: 49 - protein, 505 aa, Oryza sativa
MAAPSAIPRRGLFIGGGWREPSLGRRLPVVNPATEATIGDIPAATAEDVELAVSAARDAFGRDGGRHWSRAPGAVRAKYLKAIAAKIKDKKSYLALLETLDSGKPLDEAAGDMEDVAACFEYYADLAEALDGKQRAPISLPMENFESYVLKEPIGVVGLITPWNYPLLMATWKVAPALAAGCTAVLKPSELASLTCLELGGICAEIGLPPGVLNIITGLGTEAGAPLASHPHVDKIAFTGSTETGKRIMITASQMVKPVSLELGGKSPLIVFDDVDIDKAVEWAMFGCFANAGQVCSATSRLLLHEKIAKRFLDRLVAWAKSIKISDPLEEGCRLGSVVSEGQYQKIMKFISTARCEGATILYGGARPQHLKRGFFIEPTIITNVSTSMQIWREEVFGPVICVKEFRTEREAVELANDTHYGLAGAVISNDLERCERISKAIQSGIVWINCSQPCFVQAPWGGNKRSGFGRELGQWGLDNYLSVKQVTKYCSDEPYGWYRPPSKL

SEQ ID NO: 50 - protein, 503 aa, Oryza sativa
MATAIPQRQLFVAGEWRAPALGRRLPVVNPATESPIGEIPAGTAEDVDAAVAAAREALKRNRGRDWARAPGAVRAKYLRAIAAKIIERKSELARLETLDCGKPLDEAAWDMDDVAGCFEYFADLAESLDKRQNAPVSLPMENFKCYLRKEPIGVVGLITPWNYPLLMATWKVAPALAAGCTAVLKPSELASVTCLELADVCKEVGLPSGVLNIVTGLGSEAGAPLSSHPGVDKVAFTGSYETGKKIMASAAPMVKPVSLELGGKSPIVVFDDVDVEKAVEWTLFGCFWTNGQICSATSRLILHKKIAKEFQERMVAWAKNIKVSDPLEEGCRLGPVVSEGQYEKIKQFVSTAKSQGATILTGGVRPKHLEKGFYIEPTIITDVDTSMQIWREEVFGPVLCVKEFSTEEEAIELANDTHYGLAGAVLSGDRERCQRLTEEIDAGIIWVNCSQPCFCQAPWGGNKRSGFGRELGEGGIDNYLSVKQVTEYASDEPWGWYKSPSKL

SEQ ID NO: 51 - protein, 423 aa, Oryza sativa
MSGSERGRVMAKYAEVVERHADELAALESLDAGKPLAAARAVDVGECVGILRYFAGAADKIHGETLKMSRQLQGYTLREPLGVAGLIVPWNFPAIMFFSKVSPALAAGCTVVVKPAEQTPLSALFLAHLSKQAGVPDGVINVVTGFGPTAGAAISSHMDVDVVAFTGSTEVGRLIMEASAKSNLKPVALELGGKSPFIVFDDADLDKAVELAIGGNFFNKGEACVAGSRVFVQEGIYDRFEQKLADTMKSWVVGDPFDPRVNQGPQVDKAQYERVLGYIEQGKAEGATVLTGGKPCGKKGYYIEPTIFTNVKDDMVIAREEIFGPVMCLMKFKTVEEAIERANGTRYGLAAGLVTRDIDVANRMARSIRAGVVWVNCYFAMDRSCPFGGRKMSGFGKDDSMHALDKFLAVKSVVTPVHGSPWF

SEQ ID NO: 52 - protein, 499 aa, Oryza sativa
MGRVAPSVEEVGGEQPPPALGPGETVSGTVAELRAAYESGRTRSLEWRQSQLRGLLRLLAEEEAAAFRALREDLGKHQAEAYRDEIGVLVKSANAALREVGKWMAPEKVWVPLIAFPARAQLEPQPLGVILVFSCWNVPLGLSLEPLVGALAAGNAVALKPSELAPATAKFLGDNVGKYMDATAVKVIQGGPEVGEQLMEHRWDKVLFTGSPRIARVVMAAAAKHLTPVALELGGKCPCIFDTIGGSARDLQTAVNRVVGGKWSSCAGQACLAIDYVLVEERFVPVLIKALKSTLKKFFADSDHMARIVNARHFQRLSDLLKDKSVAASVLHGGTLDAKNLCIEPTILLNPPLDSAIMTEEIFGPLLPIITVKKIEDSIAFVRARPRPLAVYAFTKNAALRRRIVEETSSGSVTFNDAVVQYGIDSLPFGGVGESGFGQYHGKYSFEMFSHKKAVLTRGYLIELTARYPPWDDSKISMMRQLYRYNYVGFVLTFLGLKK

SEQ ID NO: 53 - protein, 491 aa, Oryza sativa
MAPAMVAAMGEKPKPAVVLGGMVSGLREVYESGRTKDLEWRQSQLKALIRLLTDKEEEIFAVLHDDLGKHRGESFRDELGILVKSIKYTLQNLKKWAASERAESPLVAFPATAMVVPEPLGVVLVFSCWNLPLGLALEPLSGAIAAGNAVVLKPSELAPSTAAFLAANIPRYLDSRAVKVVLGGPNVGEELMEHRWDKVLFTGSARIGRIIMAKAVKHLTPVALELGSKCPCIVDWLDSKRDRQIAVNRIIGAKWSTCAGQACIAIDHVIVEERFAPILIELLKSTLKRFMAKPGGMARILNAKHFERLSGYLEDNRVAASVVHGGYMDPKKLNIEPTLLLNPPADSDVMTEEVFGPILPIITVKKIEDCIAYLKSKPKPIAMYAFTNNERLKRRIVEETSSGSVTFNDAVVQYALESVPFGGVGHSGFGQYHGKYSFELFSHKKAVFKRSFLIEFMFRYPPWDERKIGTLRHVFSYNYFLLFFNLLGFRR

SEQ ID NO: 54 - protein, 484 aa, Oryza sativa
MAAARSVGMEAEVAALRGRFAAGGTRGAEWRAAQLRGILRMAAEAEAEVCRALHADLAKPYTESYVHEIALVKSSCKFALKNLKKWMKPQKVTAPLMTFPSTARVAAEPLGVVLVISAWNYPFLLSIDPIIGAIAAGNAVVLKPSEVAPATSSLLAELLPRYVDGSCIKVVEGGVAETTTLLEQKWDKIFYTGNGKVGRIVMASAAKHLTPVVLELGGKCPVVVDSNVNLHVTAKRIAAGKWGCNNGQACISPDFIITTKSFAPKLLEALEKVLEKFYGRDPLRSSDLSRIVNSNHFNRLKKLMDDENVSDKIVFGGQRDEHQLKIAPTIFMDVPLDSGIMKEEIFGPLLPIITVDKIHESFALINSMTKALAAYLFTKDSKLQEQYEAAISAGGMLVNDTAVHLTNQYLPFGGVGESGMGAYHGRFSFEAFSHKKAVLVRRFAGEAAARYPPYSPAKLKILRGVLKGNLGAMIKAILGFPRGK

SEQ ID NO: 55 - protein, 482 aa, Oryza sativa
MAEEEVAAVVGELRGSFRSGRTRAAEWRAAQLRGIVRMVEEREGDISDALHSDLAKPRMESYLHEISLAKAACTFALKGLKNWMKPEKVPAALTTFPSTAQIVSEPLGVVLVISAWNYPFLLSIDPVIGAIAAGNAVVLKPSEIAPATSALFAKLLPEYVDSSCIKVVEGGVPETTALLEQKWDKIFYTGSGNVGRIVMAAAAKHLTPVALELGGKCPAIVDSNTDLHVTMKRLAVGKWGCNNGQACIAPDYVITTKSFAPELVDSLKRVLKRFYGEDPLQSEDLSRIVNSNHFRRLTNLIEDKKVAQKIVYGGQTDEKQLKIAPTVLLDVPLDTTLMAEEIFGPLLPIVTVDKIEDSIQFINSRTKPLAAYLFTKDKKLQEEFVSNVPAGGMLVNDVALHLANPHLPFGGVGDSGIGSYHGKFSFDCFTHKKAVLIRGFGGEATARYPPYTIEKQKILRGLINGSFFALILALLGFPKERR

SEQ ID NO: 56 - protein, 527 aa, Oryza sativa
MAMAMAMRRAAALGARHILAASSTSSSGVLLRRHMSVDAGAAMEKVRAAGLLRTQGLIGGKWVDAYDGKTIEVQNPATGETLANVSCMGSKETSDAIASAHSTFYSWSKLTANERSKALRKWHDLIISHKEELALLMTLEQGKPMKEALVEVTYGASFIEYFAEEAKRIYGDIIPPTLSDRRLLVLKQPVGVVGAVTPWNFPLAMITRKVGPALACGCTVVVKPSEFTPLTALAAADLALQAGIPAGAINVVMGNAPEIGDALLQSTQVRKITFTGSTAVGKKLMAGSANTVKKVSLELGGNAPCIVFDDADIDVAIKGSLAAKFRNSGQTCVCANRILVQEGIYEKFASAFIKAVQSLKVGNGLEESTSQGPLINEAAVQKVEKFINDATSKGANIMLGGKRHSLGMSFYEPTVVGNVSNDMLLFREEVFGPVAPLVPFKTEEDAIRMANDTNAGLAAYIFTKSIPRSWRVSEALEYGLVGVNEGIISTEVAPFGGVKQSGLGREGSKYGMDEYLELKYICMGNLN

SEQ ID NO: 57 - protein, 464 aa, Oryza sativa
MSLILSRRRLAAAVRRSGPAALASRWMHTPPFATVSPQEISGSSPAEVQNFVQGSWTTSGNWNWLVDPLNGEKFIKVAEVQEAEIKPFVESLSNCPKHGLHNPLKAPERYLMYGDISAKAANMLGQPVVSDFFAKLIQRVSPKSYQQALAEVQVSQKFLENFCGDQVRFLARSFAVPGNHLGQSSNGYRWPYGPVAIITPFNFPLEIPLLQLMGALYMGNKPVLKVDSKVSIVMDQMLRLLHACGMPAEDVDFINSDGITMNKLLLEANPKMTLFTGSSRIAEKLAADLKGKIKLEDAGFDWKILGPDVQEVDYIAWVCDQDAYACSGQKCSAQSILFMHKNWSSSGLLDKMKSLSERRKLEDLTIGPVLTVTTSSMIEHMKNLLKIPGSKVLFGGEPLENHSIPEIYGAFKPTAVFVPLSEILKSGNFELVTREIFGPFQVVTEYSDDELELVLEACERMNAI

SEQ ID NO: 58 - protein, 808 aa, Oryza sativa
MASEHKLDMILRRMEEFERRRVEAEQTRRADFQSLKAALASWIPHVQNNTEDSHFLVGNKQYKMTPTMCSTECFSPNVEPNLAVDVVVTCATTSMTSVDLVSAEDAIGATYIYNPVQPMVTPAKCLTNCSNPNNIPDLTVAAVVTCTSTSLASMDLEVGKDVACTTEIDGPDCHKETHTKCSMLGLDVKGGADHVGGVFLTMSGVAKSVPISIESIDIFSARLVSDLKQDIPTPTGCLLRIPRYDSKTQFNKKLRLEGIELKPWPPPTYSGVISGLEFRPMPWPAFIYCWLEEHLLDPWPPPTEWAELELWPPPHENDILPLLINGFTHILVDRKAISKFWKAIWSELGEEWSLFVPKLYELHLSGLLQHSVSMISQQLMSRRIWSTKAKMKMLNGWDSKQYLSIMRPIPGLFVKLIQDISPKSHHQAYIEAQVAKKFLENFGEDKVHFLAQSIAVPDTHLGQGCIGCCWLYGPNAISTFHLLIAILHIVRPLYMENIFTRTSHVAKKWAKDLKGVVFDWNTLGIVVQEVNYIELICDRDSYGCSVQKCYPQSILFMHNGWSFGGLLDWNSKQYKNSMLIVNPLELMQVLLVPLVWDPDAEMGQIGSYALQPENCQLTTCMRAHCIKPSDYEIITAKENHAADAPRLLIVVMEDEGNTVWTLEFSVSGVVQKKRDLWLCAFAVNKDMKVVMFLQQYGYANLVIVNLLSVPWDPGGSHLALAIKQGTRLSLWAITSIGWLCFLWSYWLHYKRNSNRGDQVGEACTASSHRLGDKPNFKGRRMLGAMWAAIWAGSATFQEVQASPRGNHI

SEQ ID NO: 59 - protein, 716 aa, Oryza sativa
MASVDPSRSFVRDVKRVIIKVGTAVVSRQDGRLALGRVGALCEQVKELNSLGYEVILVTSGAVGVGRQRLRYRKLVNSSFADLQKPQMELDGKACAAVGQSGLMALYDMLFNQLDVSSSQLLVTDSDFENPKFREQLTETVESLLDLKVIPIFNENDAISTRKAPYEDSSGIFWDNDSLAGLLALELKADLLILLSDVDGLYSGPPSEPSSKIIHTYIKEKHQQEITFGDKSRVGRGGMTAKVKAAVLASNSGTPVVITSGFENRSILKVLHGEKIGTLFHKNANLWESSKDVSTREMAVAARDCSRHLQNLSSEERKKILLDVADALEANEDLIRSENEADVAAAQVAGYEKPLVARLTIKPGKIASLAKSIRTLANMEDPINQILKKTEVADDLVLEKTSCPLGVLLIVFESRPDALVQIASLAIRSGNGLLLKGGKEAIRSNTILHKVITDAIPRNVGEKLIGLVTTRDEIADLLKLDDVIDLVIPRGSNKLVSQIKASTKIPVLGHADGICHVYIDKSADMDMAKHIVMDAKIDYPAACNAMETLLVHKDLMKSPGLDDILVALKTEGVNIYGGPIAHKALGFPKAVSFHHEYSSMACTVEFVDDVQSAIDHIHRYGSAHTDCIVTTDDKVAETFLRRVDSAAVFHNASTRFSDGARFGLGAEVGISTGRIHARGPVGVEGLLTTRWILRGRGQVVNGDKDVVYTHKSLPLQ

SEQ ID NO: 60 - protein, 735 aa, Oryza sativa
MGRGGIGGAGLVAAVAKADVENTDSTRGFVKDVKRIIIKVGTAVVTGPNGRLAMGRLGALCEQVKQLNFEGYEVILVTSGAVGVGRQRLKYRKLVNSSFADLQNPQMDMDGKACAAVGQSVLMAIYDTLFSQLDVTSSQLLVTDRDFMDPSFGNQLRETVNSLLDLKVIPVFNENDAISTRRQPYEDSSGIFWDNDSLARLLAQELKADLLIMLSDVEGLYSGPPSDPQSKIIHTYVHEQHGKLISFGEKSRVGRGGMQAKVAAAFTASSKGIPVVIASGFAIDSIIKVMRGEKIGTLFHREANQWGCSKEATAREMAVAARDCSRHLQKLSSEERKKILLDIADALEANEDLITSENQADLDLAQDIGYDKSLVARMTIKPGKIKSLAGSIREIADMEDPISHTLKRTEVAKDLVFEKTYCPLGVLLIIFESRPDALVQIASLAIRSGNGLLLKGGKEAMRSNTILHKVITGAIPDVVGKKLIGLVKNKDEIADLLKLDDVIDLVIPRGSNKLVSQIKAATKIPVLGHADGICHVYIDKSADMDMAKRIVLDAKVDYPAACNAMETLLVHKDLNRTEGLDDLLVELEKEGVVIYGGPVAHDTLKLPKVDSFHHEYNSMACTLEFVDDVQSAIDHINRYGSAHTDCIITTDGKAAETFLQQVDSAAVFHNASTRFCDGARFGLGAEVGISTGRIHARGPVGVDGLLTTRCILRGSGQVVNGDKGVVYTHRELPLQ

SEQ ID NO: 61 - DNA, 1527 bp, Euphorbia characias
atgggttttgcgaggaaagagtatgaatttctgagcgagattggtttgagcgaacgcaacttgggttgttatgtcaatggcacctggaaagctaatggtcctgtggtcaccacttcgaatcctgcaaacaaccaagcaatagctgaagttgtggagggttcgatcgaggattatgaagaaggcatgaaagcatgcagtgaagcagcaaagatatggatgcaggttcctgctccaaagagaggtgatattgttagacagattggtgatgcgctaagggggaagcttgagcaccttggtcgcctcgtctcacttgagatggggaaaatacttgctgaaggcatcggtgaagttcaagaaatcatcgacatgtgtgatttttgtgttggattaagccgacagctaaatggctctataataccttcagaacgtccaaatcatgcaatgttggagatgtggaatcctttagggattgtcggtgttataacggctttcaacttcccatgtgctgttctcgggtggaatgcatgcatagcactggtctgcggcaattgcgctgtgtggaaaggtgcaccaacaacacctttgatgaccatagctacgacaaagctggtagctgaggtgttagagaggaacaacttaccgctggcaatttttacatctttttgcggtggtgctgatattggccaagcaatagcaaaggacacacgtattcctctggtttcattcactgggagttcaaaggtgggccttatggttcaacaaacagtgaatcaaagatacggtaaatcgttgctagaattaagcggaaacaatgcaataatagtgatggatgacgctgacatccctctagctgcccgttcaatactgttcgctgctgttgggacagctggacagcggtgcacaacttgccgtaggctgatccttcatgagaaaatatacgacacggtgcttgatcaactattgaaatcatacaaacaagttaagatcggagatccactggaaaagggtactttgctcggtccagtgcatactgctgaatcaaggaagaattttgagaagggaatagagctaattaagtcccagggagggaagattctaacaggcggttcagttatagagtctgaggggaattatgtacagccgacaatagttgagatatcttcaaaagccgaagttgttaaggaagagttatttgctcctgttctttatgttatgaaatttcagactttagaagaagccattgaaataaataattcagtgccccagggattaagtagttccatcttcacccgtagacctgatgttatcttcaagtggcttgggccacatggaagtgactgtggcatcgtgaatgtaaatataccgacaaatggtgctgaaattggtggtgcttttggtggagagaaggcaactggtggcggccgtgaagcaggaagtgactcctggaagcagtatatgcgagcctccacatgcacaatcaactatgggagcgaattaccactagcacaaggtattaacttcggctag

SEQ ID NO: 62 - protein, 507 aa, Euphorbia characias
MGFARKEYEFLSEIGLSERNLGCYVNGTWKANGPVVTTSNPANNQAIAEVVEGSIEDYEEGMKACSEAAKIWMQVPAPKRGDIVRQIGDALRGKLEHLGRLVSLEMGKILAEGIGEVQEIIDMCDFCVGLSRQLNGSIIPSERPNHAMLEMWNPLGIVGVITAFNFPCAVLGWNACIALVCGNCAVWKGAPTTPLMTIATTKLVAEVLERNNLPLAIFTSFCGGADIGQAIAKDTRIPLVSFTGSSKVGLMVQQTVNQRYGKSLLELSGNNAIIVMDDADIPLAARSILFAAVGTAGQRCTTCRRLILHEKIYDTVLDQLLKSYKQVKIGDPLEKGTLLGPVHTAESRKNFEKGIELIKSQGGKILTGGSVIESEGNYVQPTIVEISSKAEVVKEELFAPVLYVMKFQTLEEAIEINNSVPQGLSSSIFTRRPDVIFKWLGPHGSDCGIVNVNIPTNGAEIGGAFGGEKATGGGREAGSDSWKQYMRASTCTINYGSELPLAQGINF

SEQ ID NO: 63 - DNA, 1527 bp, Thelungiella halophila
atgggttctgcgaacaaagagtacgagtttctgagtgagattgggttgagttctcacaacctcggaagttacgttggtggcaaatggcaaggaaacggacccgttgtctctactctcaatcccgccaacaatcagccaattgctcaagttgttgaagcttctctggatgattacgagaaaggtttgaaagcttgcgaggaagcagctaaaacatggatgcaggttcctgctcccaagagaggtgatatcgtgagacaaattggagatgcactaagatccaaacttgactatcttggccgtcttctttcacttgaaatggggaagatccttgctgaaggaattggagaagttcaggaagtaattgacatgtgtgattttgctgttggtttgagccgacaactcaatggatcagtcataccttcagaacgcccaaatcacatgatgttggagatgtggaatccgcttggcattgtaggtgttatcacagcgtttaactttccatgtgcagttcttggttggaatgcttgtattgcactggtctgcggaaactgtgtagtctggaaaggtgctccaactacaccgttgataactatcgcaatgactaagctcgtggccgaagttttagagaagaacaatttacccggtgccatttttacggccatgtgtggtggtgctgagattggtgaagcaatagccaaagacacacgcattcctctagtatcctttactggaagctccaaggtgggcttaaaggtacaacaaacagtgagtgcgagatctggcaaaactctgcttgaattgagtggaaacaatgcaatcatagtcatggaagatgctgacatacagttagctgctcgttctgttctgtttgctgcggttggaaccgctggtcaacgttgcacaacttgccgtaggctgcttttgcatgagagggtctatgataaagtgctcgagcaactccttacttcatacaaacaagtcaaaatcggcgatcctcttgaaaaagggacattgttaggaccactacatactcctgaatcaaagaagaactttgagaaaggaatcgaagtcatcaaatcccagggcggtaaaatactaacgggcggtaaagcaatcgaaggtgaaggaaactttgtggagcctacgataatcgagatatcatcagatgcatctgttgttaaagaagagctatttgctcctgttctatatgctctaaaatttaagtcatttgaagaagctgttgcgataaacaactcggttcctcaaggtctaagcagctcgatattcactcgtaatcccgaaaacatctttaagtggatcggaccaatgggaagtgattgtggcattgtgaatgtgaacatacctacgaacggagctgagatcggaggagcgtttggaggcgagaaagcgacaggtggtggtcgtgaagctggaagcgattcatggaagcaatacatgcgtcgttcaacttgtacgatcaactatgggaacgagttacctctagcgcaaggaattaacttcggttaa

SEQ ID NO: 64 - protein, 508 aa, Thelungiella halophila
MGSANKEYEFLSEIGLSSHNLGSYVGGKWQGNGPVVSTLNPANNQPIAQVVEASLDDYEKGLKACEEAAKTWMQVPAPKRGDIVRQIGDALRSKLDYLGRLLSLEMGKILAEGIGEVQEVIDMCDFAVGLSRQLNGSVIPSERPNHMMLEMWNPLGIVGVITAFNFPCAVLGWNACIALVCGNCVVWKGAPTTPLITIAMTKLVAEVLEKNNLPGAIFTAMCGGAEIGEAIAKDTRIPLVSFTGSSKVGLKVQQTVSARSGKTLLELSGNNAIIVMEDADIQLAARSVLFAAVGTAGQRCTTCRRLLLHERVYDKVLEQLLTSYKQVKIGDPLEKGTLLGPLHTPESKKNFEKGIEVIKSQGGKILTGGKAIEGEGNFVEPTIIEISSDASVVKEELFAPVLYALKFKSFEEAVAINNSVPQGLSSSIFTRNPENIFKWIGPMGSDCGIVNVNIPTNGAEIGGAFGGEKATGGGREAGSDSWKQYMRRSTCTINYGNELPLAQGINFG

SEQ ID NO: 65 - DNA, 1536 bp, Picea sitchensis
atgggagcggactttgtaagaaatgaatatcagtttttgaagggtcttggcttacattcccataacctgggatgttatggcaatggaatctggaaaggaagcggcaatactgtgacatcagtaaatccttccaataaccagccaatagcaacggtagttgaagcttctattgaagagtatgaagatagcttgagagcctgtgctgaagctcgtaaaatatggatgcaaactcctgcaccaaagagaggtgaaattgtaaggcagattggggaagctttgcggtcaaaattggatctgcttggtagacttgtatcattggagatgggaaaaatccttgcagaggggataggagaggttcaagaatttattgacatgtgtgattatgcggttggcttaagtcgacagttgaatggctctatcataccttcagaacggcccaatcatatgatgatggaggtttggaatcctcttggaatagtcggagtaattactgcattcaactttccatgtgctgtacttggatggaatgcatgcatcgctttggtttgtggcaattgcgttgtttggaaaggtgctcctactacaccattgatcactatagcaacaacaaaactcatagccgaggtgttagagagaaatggttttcctggtgccattttcacctccatctgtggtggtgcagagattgggcaggcgatagctgaagatggaagaatcccattggtctcttttactggaagcacgaaggttggacagattgttcagaacgcagtcaatgcaagatttgggaagtgtttgctcgagcttagtggaaacaatgccattatagttatggaagatgcagacttagcaatggttgtgcgatctgttttgtttgctgcagttggcactgcaggacaacgatgcaccagttgccgaaggctgttactccacgagaaggtataccagcatgtcattgagcagttgactgatgcatataagcaagttaaattgggagaccctcttgataaagataccttgttaggaccactgcatactcgtgcctcaaagattaactttgagaaaggaattcaagttataaaatctcagggtgggcgcattctgacaggcggctcagttatagagcatgaagggaactttgtgcagcctacgattgtggaaatatctaaagatgcaccagttgtgaaggaagagctttttggacctgttctctatgtcttgaagtttcagaccttggaggaagctattgaaataaacaattctgtttctcaaggcctaagtagttccatcttcactcggagtccagaagctatttttaagtggattggaccttgtggtagtgattgtggtattgtaaacataaatataccgactagtggggcagagattggaggtgcatttggtggtgaaaaggcaacaggtggtggtcgtgaagcaggaagtgattcatggaaacagtacatgcatcgtgcaacttgtacactcaattatgggagcgaactgccactagcacaagggattaattttggatgttaa

SEQ ID NO: 66 - protein, 511 aa, Picea sitchensis
MGADFVRNEYQFLKGLGLHSHNLGCYGNGIWKGSGNTVTSVNPSNNQPIATVVEASIEEYEDSLRACAEARKIWMQTPAPKRGEIVRQIGEALRSKLDLLGRLVSLEMGKILAEGIGEVQEFIDMCDYAVGLSRQLNGSIIPSERPNHMMMEVWNPLGIVGVITAFNFPCAVLGWNACIALVCGNCVVWKGAPTTPLITIATTKLIAEVLERNGFPGAIFTSICGGAEIGQAIAEDGRIPLVSFTGSTKVGQIVQNAVNARFGKCLLELSGNNAIIVMEDADLAMVVRSVLFAAVGTAGQRCTSCRRLLLHEKVYQHVIEQLTDAYKQVKLGDPLDKDTLLGPLHTRASKINFEKGIQVIKSQGGRILTGGSVIEHEGNFVQPTIVEISKDAPVVKEELFGPVLYVLKFQTLEEAIEINNSVSQGLSSSIFTRSPEAIFKWIGPCGSDCGIVNINIPTSGAEIGGAFGGEKATGGGREAGSDSWKQYMHRATCTLNYGSELPLAQGINFGC

SEQ ID NO: 67 - DNA, 1551 bp, Tortula ruralis
atgagggttggtgcaatggccgtgaaatttgaaagggaggattatcagttcctgacggagctggggcttgctcctgagaatttgggatgctatggaggtggtgtgtggcgagcgaatggccccactgtcacgaccatcaacccatccgataacgagccgatcgcaactgtgagggaagcttcacttgaagactatgaagacagcatgaaggcctgtgcggacgctcgccggatgtggatgctgacgccagcaccaaagaggggtgagatagtgcgacagattggggatggactccgtgcgaagctcccgctcctgggcaagcttgtgtctcttgaaatgggcaagatcctacctgaaggcattggggaagtccaggagttcatcgacatgtgtgattacgcagttgggttgagtcggcagctcagtggttccataattccttctgaacgtccaaatcacgccatgatggaggtgtggaatccattgggtatagtcggagtgattactgcgttcaattttccttgcgccgtactcggatggaatgcgtgtattgcacttgtttgtggcaactgcgttgtatggaagggagcaccaacaaccccactgttgacgcttgctacgaccagaatcatcgcagaggttttcgaaaggaacaaactacctggggggatttttacttgcgcttgtgggggtgctgaaatcggtagcgccattgcacatgatgcccgcataccgctcgtgtctttcactggcagtactaaggtgggaatgctggtgcaaagtattgtgcatgcaaggcatgggaaggtgttgttggagctgagcgggaacaacgccatcatagtgatggacgatgcggtcttgcctcttgccatacgtgctgtcttgttcgccgctgttggcactgctgggcagcgttgcaccacctgccggcgcctgatcgtacatgagaaagtttatgacgatatgctggagggcctgctcaaggcatacaagcaggtcaaagtcggcaactctgtggaaggcgatacgttgtgtggaccccttcattccaagctttccatgtcaggctttacagagggcatcaagaagatcaaggcccaggggggtaacatcttgactggcggctcaacaattaatcgtgctggaaattttgttgaaccaaccctggtcgaaatctcacatgatgcagaagttgtgagggaagagttgttcggccctgtgttgtatgtctttaaaatcaagtcgttggaggaagcgattgagatgaacaactcagtgccacagggtctcagcagctcaatcttcacacgtaacccagaaacaatcttcacttggattgggcccacaggcagtgactgtggtattgtgaatgtaaatataccaaccaatggtgcggagattggaggagcctttggtggagaaaaagcaacgggaggcggaagggaggcgggtagcgattcgtggaaacagtacatgcaccgtgcaacctgtactatcaattatggcaaggatctaccgttggctcaaggcataaacttcggaggttga

SEQ ID NO: 68 - protein, 516 aa, Tortula ruralis
MRVGAMAVKFEREDYQFLTELGLAPENLGCYGGGVWRANGPTVTTINPSDNEPIATVREASLEDYEDSMKACADARRMWMLTPAPKRGEIVRQIGDGLRAKLPLLGKLVSLEMGKILPEGIGEVQEFIDMCDYAVGLSRQLSGSIIPSERPNHAMMEVWNPLGIVGVITAFNFPCAVLGWNACIALVCGNCVVWKGAPTTPLLTLATTRIIAEVFERNKLPGGIFTCACGGAEIGSAIAHDARIPLVSFTGSTKVGMLVQSIVHARHGKVLLELSGNNAIIVMDDAVLPLAIRAVLFAAVGTAGQRCTTCRRLIVHEKVYDDMLEGLLKAYKQVKVGNSVEGDTLCGPLHSKLSMSGFTEGIKKIKAQGGNILTGGSTINRAGNFVEPTLVEISHDAEVVREELFGPVLYVFKIKSLEEAIEMNNSVPQGLSSSIFTRNPETIFTWIGPTGSDCGIVNVNIPTNGAEIGGAFGGEKATGGGREAGSDSWKQYMHRATCTINYGKDLPLAQGINFGG

SEQ ID NO: 69 - DNA, 1542 bp, Setaria italica
atgggggcctttgcgaggaaggagcaccagttcctctccgagctcggcctcgcgccgcgcaacctgggatccttcgcctgcggggcctggggcggctcgggccccgtcgtcaactccaccagcccggccaacaaccaggttatcgcggaggttgtggaggcgtccgtgaacgactacgaggatggcatgcgcgcctgcttcgatgcctccaagtcatggatggcgattcctgctccgaagcggggagagattgtccggcaaattggtgatgcactgagagcaaagctccatcaccttggcaggcttgtgtcacttgagatggggaaaattcttccggaagggatcggggaggttcaggaaatcattgacatgtgtgattatgctgtggggctaagtcgccaattaaatggatcaattataccatctgaacgagccaggagaggtccgaaccatatgatgatggaggtgtggaatccacttggagtcgtcggtgttataacagcatttaattttccttgcgctgtgctcggttggaatgcttgcattgctttggtctgcggaaactgtgttgtctggaaaggtgccccaactactccattgatcactattgcaatgactaaaatagtagctagtgtgttggagaagaacaacttgccaggcgcaattttcacagctttttgtgggggcactgaaattggccaagcaatcgctgttgacactaggatacctttggtttcgttcactggaagtacaagggctggtctaatggttcagcaacaagttaatgcaagatttggtaaatgccttcttgaacttagcgggaacaatgccataattgttatggatgacgcagatattcagctagccgtgcgttctgtgttgtttgctgctgttggtacagcaggacaacgctgcactacatgccgtaggctgattcttcatgaaagtatatatcaaacatttcttgatcaacttgttgaggtatataaacaagtcagaattgggcatccgttggagaagggcaccttactgggaccgctgcacactcctacttcaaaagagaactttttgaaaggcatacagaccatcaaatctcagggagggacaatcctttttggaggatctgccattgaatcagaagggaacttcgtacagccaacgattgtggaaatttcagcttctgcaccagttgtgaaagaagaactctttggccctgtcctttatgtgatgaaatttcagaccttgaaggaagcaattgaaatcaataactctgttcctcaaggattgagcagttcaatatttacaaagaggccagaaattatttttaagtggcttgggccccatggtagtgactgtggtatagtgaatgtgaatatacctaccaacggtgctgaaatcggtggagcatttggtggagaaaaagcaactggtggtggacgagaagcaggaagtgattcctggaagcagtacatgaggagggcaacttgtacaatcaactatggaaccgagctacctctagcacagggaatcaattttggttaa

SEQ ID NO: 70 - protein, 513 aa, Setaria italica
MGAFARKEHQFLSELGLAPRNLGSFACGAWGGSGPVVNSTSPANNQVIAEVVEASVNDYEDGMRACFDASKSWMAIPAPKRGEIVRQIGDALRAKLHHLGRLVSLEMGKILPEGIGEVQEIIDMCDYAVGLSRQLNGSIIPSERARRGPNHMMMEVWNPLGVVGVITAFNFPCAVLGWNACIALVCGNCVVWKGAPTTPLITIAMTKIVASVLEKNNLPGAIFTAFCGGTEIGQAIAVDTRIPLVSFTGSTRAGLMVQQQVNARFGKCLLELSGNNAIIVMDDADIQLAVRSVLFAAVGTAGQRCTTCRRLILHESIYQTFLDQLVEVYKQVRIGHPLEKGTLLGPLHTPTSKENFLKGIQTIKSQGGTILFGGSAIESEGNFVQPTIVEISASAPVVKEELFGPVLYVMKFQTLKEAIEINNSVPQGLSSSIFTKRPEIIFKWLGPHGSDCGIVNVNIPTNGAEIGGAFGGEKATGGGREAGSDSWKQYMRRATCTINYGTELPLAQGINFG

SEQ ID NO: 71 - DNA, 1533 bp, Glycine max
atgggttccgataatcaccagaacttggagttcttgaaggagatcggtttgggctcttccaacattggctcttacattaatggccagtggaaagccactggttcttctgtcacttctgttaatccttctaacaatcagagtatagctcaagtgactgaagcaactttgcaagattttgaggaggggttgcgagcttgcagtgaagcagctaagacatggatgactataccggcaccgaagagaggtgagattgtgagacagattggtgaagcattgagggccaaattggatcctttgggtaggctggtgtctcttgagatgggaaaaattctcccagaaggaattggggaagttcaggaaattattgatatgtgtaattatggtgttgggctaagcagacaattgaatggatcaattataccatcagaacgtccagatcatatgatgtttgaggtatggaacccactaggaatagttggtgtaatcagtgctttcaactttccatgcgctgttctaggatggaatgcttgcattgctttagtctgtggtaactgtgttgtgtggaagggtgctccaacaactcctttgataactattgctgtgacaaagctagtagctgaagttcttgagaggaacaaattacctggtgcaatattcacctctttctgtggaggtgctgacattggtcaggcaatagcaaaagacactcgcattcccctggtttcatttactggaagttcaaaggttggcttgatggtccagcaaacagttaatgagagatttggcaaatgcttgcttgagttaagtggtaacaatgcaataattgtcatggatgatgcagactcaaattggctgtacgtctattttggttgctgctgtgggtccactggtcaaccggtgtacacttggccttgtcgtagactgtttctgcatgaaagtatttatacagacgtactagaccaacttgttgaagtctacaaacaagtcaaaactgggaaccccttggagaaggggactctagttgggcccttgcatactcgtacttcagtagaaaactttcagaagggtatttcagtcataaaatctcagggagggaaaatcctaaccggtggatctgtattagagtcaggaggaaattttgtacaaccaacaattgttgagatttctccagatgctcctgtagttaaagaagaattgtttggtccagttctgtatgtgatgaaatttcagactctagacgaagcaattgccttgaacaattctgtacctcaaggattaagtagttcaatctttacccaaagacctggaactatattcaaatggatcgggccacgaggtagtgattgtggtatagtgaatgcaaacatacctacaaatggagctgaaattggtggtgcctttggtggagaaaaggccacaggtggtggccgtgaagcgggaagtgactcatggaagcaatacatgcggcgttctacatgtaccatcaattatggaagtgaactaccattagctcaggggataaactttggctag

SEQ ID NO: 72 - protein, 510 aa, Glycine max
MGSDNHQNLEFLKEIGLGSSNIGSYINGQWKATGSSVTSVNPSNNQSIAQVTEATLQDFEEGLRACSEAAKTWMTIPAPKRGEIVRQIGEALRAKLDPLGRLVSLEMGKILPEGIGEVQEIIDMCNYGVGLSRQLNGSIIPSERPDHMMFEVWNPLGIVGVISAFNFPCAVLGWNACIALVCGNCVVWKGAPTTPLITIAVTKLVAEVLERNKLPGAIFTSFCGGADIGQAIAKDTRIPLVSFTGSSKVGLMVQQTVNERFGKCLLELSGNNAIIVMDDADSNWLYVYFGCCCGSTGQPVYTWPCRRLFLHESIYTDVLDQLVEVYKQVKTGNPLEKGTLVGPLHTRTSVENFQKGISVIKSQGGKILTGGSVLESGGNFVQPTIVEISPDAPVVKEELFGPVLYVMKFQTLDEAIALNNSVPQGLSSSIFTQRPGTIFKWIGPRGSDCGIVNANIPTNGAEIGGAFGGEKATGGGREAGSDSWKQYMRRSTCTINYGSELPLAQGINFG

SEQ ID NO: 73 - DNA, 1527 bp, Pisum sativum
atgggttctgatagcaacaatttgggattcttgaaggagatcggtttgggtgctactaacatcggttctttcatcaatggccaatggaaagccaacggtcctactgttcattccgtcaatccttccaccaatcaggttattgcttccgtgactgaagcaactttggatgattatgaagagggattgagagctagcagtgaagcagcaaagacatggaggactgttccggcaccgaaaagaggtgagattgtaagacagattggggatgcgttgagggctaagttggatccacttggtaggttggtggctcttgagatggggaaaattcttgctgaaggaattggtgaggttcaggaaattattgatatgtgtgattattctgttgggctgagccgacaactgaatggatcaattatcccatcagaacgtccagagcatatgatgtttgaggtatggaacccattaggaatcgttggtgtaatcactgctttcaactttccatgtgctgtactaggatggaatgcttgcattgcactagtcggtggtaacactgttgtttggaagggcgcgccaactactccattgataactgttgctgtgacaaagctgattgctgaagtttttgagaggaacaatttacccggagcaatatttaccgctctctgtgggggtgctgatattgggcacgcaatagcgaaagacacacgcattcctttggtttcatttactggaagctcaaaggtgggcgcgctggtccagcaaacagtgagtcaaagatttggcaaaaccttgcttgagttgagtggtaacaatgcaataatagtcatggatgatgccgatatcacattggctgtgcgctctattttctttgcagctgtcggtactgctggtcagcgttgtacaacctgtcgtagactgtatctgcatgagagtgtttatgcaaacgtgcttgaacaacttaccgcactctacaaacaagtcaaaattgggaatcctctggaggaagggacactagttgggcctctgcatactcgttctgcggtggaaaattttaagaatggtatttctgcaattaaatctcagggagggaagattgtaacaggggggtctgtattagagtcggaaggaaactttgtcgtgccaacgattgttgagatttctgcagatgctgctgtagttaaagaagaattgtttgctccagttctctatgttatgaaatttaaggatcttgaagaagcaattgccttgaacaattccgtacctcaaggattgagtagttccatcttcacacaaaagccttctactatattcaaatggatagggccaagtggaagtgattgtggtattgtaaatgtgaacataccaacaaatggagctgagattggaggtgcctttggtggagaaaaggcaactggtggtggtcgtgaagctggaagtgactcgtggaagcaatacatgcgccgttctacatgtaccatcaattatggaagtgaattgccattagctcagggaattaactttggctaa

SEQ ID NO: 74 - protein, 508 aa, Pisum sativum
MGSDSNNLGFLKEIGLGATNIGSFINGQWKANGPTVHSVNPSTNQVIASVTEATLDDYEEGLRASSEAAKTWRTVPAPKRGEIVRQIGDALRAKLDPLGRLVALEMGKILAEGIGEVQEIIDMCDYSVGLSRQLNGSIIPSERPEHMMFEVWNPLGIVGVITAFNFPCAVLGWNACIALVGGNTVVWKGAPTTPLITVAVTKLIAEVFERNNLPGAIFTALCGGADIGHAIAKDTRIPLVSFTGSSKVGALVQQTVSQRFGKTLLELSGNNAIIVMDDADITLAVRSIFFAAVGTAGQRCTTCRRLYLHESVYANVLEQLTALYKQVKIGNPLEEGTLVGPLHTRSAVENFKNGISAIKSQGGKIVTGGSVLESEGNFVVPTIVEISADAAVVKEELFAPVLYVMKFKDLEEAIALNNSVPQGLSSSIFTQKPSTIFKWIGPSGSDCGIVNVNIPTNGAEIGGAFGGEKATGGGREAGSDSWKQYMRRSTCTINYGSELPLAQGINFG

SEQ ID NO: 75 - DNA, 1527 bp, Arabidopsis thaliana
atgggttcggcgaacaacgagtacgagtttctgagtgagattgggctgacttctcacaacttgggatcttacgttgctggcaaatggcaagccaacggacctcttgtttcaactctcaatcctgctaacaatcagccaattgctcaagttgtggaagcttctctagaggattacgagcaaggtttgaaagcttgcgaggaagcagctaaaatatggatgcaggttacggctcctaagagaggtgatatcgtcagacagattggggatgcactaagatccaaacttgactatcttggtcgtcttctttctcttgaaatgggaaagatccttgctgaaggtattggagaagttcaggaagtaattgacatgtgtgattttgctgttggtttgagccgacaactcaatggatcggttataccttcagaacgccctaaccacatgatgttggagatgtggaatcctcttggcattgttggtgttatcacagcgtttaattttccctgtgcagttcttggttggaatgcttgtattgctcttgtctgtggaaactgtgtagtctggaaaggtgctccaactacaccgttaataactatcgcaatgaccaagctagtagctgaagttttagagaaaaacaacttacccggtgccatttttacggccatgtgtggtggtgctgaaattggtgaagcaatagccaaagacacacgcattcccttagtatcctttactggaagctccagggtgggctcaatggtacaacaaacagtgaatgcgagatctgggaaaactttgcttgagttgagtggaaacaatgcaatcatagtcatggacgatgctgatatacagttagcggctcgatctgttctattcgctgcggttggaactgctggtcaacgttgcacaacttgccgtaggctgcttttgcatgagagtgtctatgacaaagtactcgagcaactgcttacctcatacaaacaagtcaaaatcggcaatcctcttgagaaagggacattgttaggaccattacatactcctgaatcaaaaaagaactttgagaaaggaattgaagtcatcaaatcccagggtggtaaaatactaacgggaggtaaagcagtcgaaggtgaaggaaactttgtggagcctacgataatcgagatatcagcagatgctgctgtcgtcaaagaagagctatttgctccggttctatatgttctaaagtttaagtcatttggagaggctgttgcgataaacaactcggttcctcaaggtctaagcagctcgatattcactcggaaccctgaaaacatcttcaggtggatcggaccactgggaagtgactgtggcattgtgaatgtgaacataccgacgaatggagctgagattggtggagcttttggaggcgagaaagcgactggtggtggtcgtgaagctggaagcgactcatggaaacagtacatgcgtcgatcgacttgtaccatcaactatggaaacgagttacctctagcgcaaggaattaacttcggttag

SEQ ID NO: 76 - DNA, 1653 bp, Arabidopsis thaliana
atgacgaagcttctagagattaatcatattcaaaccctttgttttgccaagggtttttctcctgccagattaaatgttgcaacttcaccttttcggatatctcgtcgtggtggtggtggatattgcagcaatgcttgtatcccttatcggttgaaatttacttgttatgcaacactatctgctgtggtaaaggagcaagcatcagactttagtggaaaagaagcagctttgcttgtagatgagctcagaagcaacttcaactctggcagaacgaagagctacgagtggagaatttctcagcttcaaaacattgctaggatgattgatgagaaggagaaatgcatcaccgaagctttgtatcaagatctttctaagcctgagcttgaagcttttctagctgagatttcgaatacaaaatcatcctgtatgcttgcaatcaaagagttaaagaactggatggctccagaaacggtcaaaacttctgtgacaacatttccctcgtctgcacaaatagtctcagaaccgcttggagttgttttggttatttcagcctggaatttccctttcttattgtctgtcgagccagtcattggagctattgcagctggtaatgcggttgtgctaaaaccttctgaaattgctccagcagcatcttctcttttagcaaagttgtttagtgaatacttggacaatacaacaatcagagttatcgagggaggagtccctgaaacaacggcactactggatcaaaaatgggacaagatctttttcactggtggagcaagagttgctcgcattataatggctgcggctgcgagaaatcttactccagtggttctcgaacttggtgggaagtgcccagctcttgttgattcagatgttaatctacaagtggctgctaggaggatcattgcagggaaatgggcttgtaacagtggacaggcttgcattggtgttgattacgtgatcacaacaaaggattttgcatcaaaattgatagatgctctgaagactgaactggagacattttttgggcaaaacgcattagaatcaaaggacctgtcgcggattgtgaactctttccacttcaaaaggttggaaagtatgttaaaagagaatggagtagccaacaaaattgttcatggtggccgaataaccgaagataaacttaaaatatcaccaacaatactactggatgtgcctgaagcatcttccatgatgcaggaagagatatttggacctttacttcccataattacggtacaaaagattgaagatggctttcaggttatacgttcaaagccaaagcctttagcagcgtatctcttcacaaacaacaaagagttggagaaacaattcgtgcaggacgtatcagcaggaggaattaccatcaatgacactgtcttacacgtgactgtaaaagatctgccatttggtggggttggggagagtgggatcggtgcttaccatgggaaattctcgtatgagacgtttagccataagaaaggagttctttaccggagcttttcaggggacgcggatcttagatatcctccttacacaccaaaaaagaagatggtgcttaaggctttgctctcttcaaacatatttgctgccattttggctttctttgggttctctaaagactcatga

SEQ ID NO: 77 - protein, 513 aa, Nitrosococcus oceani
MKLLKDLGLEDFNPGVCWGPGWWSGADSRRRIDSSNPATEKPIASVGAATAADVETLIGASWENFRTWRAVPAPVRGDLVRRLGESLRVHKDRLGSLVSLETGKIKEEGDGEVQEMIDMADFAVGQSRMLYGKTMHSERPSHRMYEQWHPLGPVGVITAFNFPVAVWAWNALIAAICGNTVIWKPSPKAPLTAVAVQHLCNRVMEEAGYPGVFNLLVTDENPLAESLVQDRRIPLISFTGSTKVGRWVSRLVAARLGRSLLELSGNNAVIVDETADLDLAVPAVVFGAVGTAGQRCTTTRRLIVHENCYEELISRLIHAYRQLPIGDPLDRKTLVGPLIDAEAVAKFSDAIATLKQRGGEILYGGRVLERGGYFVEPTLVRAENHWEMVQRETFAPILYLIPFKTLEEAIALNNAVPQGFSSSLFTTHLQHAERFLSHWGSDCGIANINMGTSGAEIGGAFGGEKETGGGREAGSDAWKNYMRRQTNTINWGTELPLAQGIRFELEGESPPER

SEQ ID NO: 78 - DNA, 1542 bp, Nitrosococcus oceani
atgaaattacttaaggatttgggtctagaagatttcaatccgggggtgtgttgggggccggggtggtggtctggagcggattcccgtcggcgaatcgattccagcaacccagccactgaaaagccgattgctagtgttggggcagcgaccgcggccgatgtggaaaccctcataggtgcctcctgggagaattttagaacctggcgtgccgtccccgcgccggtacggggagatttggtgcgccgcctgggtgagtctctgcgggtccataaagatcgcctgggcagcctggtgagcttggagacgggtaagatcaaggaagaaggggatggggaagtacaggaaatgattgatatggcggattttgccgtggggcaatcccggatgctctatggcaagaccatgcactcggaacgtcccagccatcggatgtatgaacaatggcatcccttaggaccggtaggggtgattaccgcctttaattttccggttgccgtatgggcctggaatgccctgattgccgctatttgcggcaatacagtcatttggaagccctctcccaaggcgcccttaacggccgtcgctgtgcagcacctttgtaatcgagtgatggaggaagccggttatccaggggtgtttaacctcttggtgaccgatgagaatccactggctgaaagtttggtgcaagaccggcggattcctttgatttcttttactggctccaccaaagtaggacggtgggtgagtcggttagtggctgcgcggttgggacgaagcctgctggaactctctggcaataacgcagtgattgtcgatgaaaccgctgatctcgacttggcagtgccggcggtagtttttggggcggtcggcacggcggggcagcgttgcaccaccacacggcgtctgattgtgcatgaaaactgctatgaggagctgatttcccggctaatccatgcttaccggcaattgcccattggcgatcccttagatagaaaaaccttagtgggaccccttattgatgccgaggccgtggcaaagtttagcgatgccatagcaacgcttaagcagcggggaggtgaaattctctacggtggccgtgtgcttgagaggggtggatattttgtcgagcctaccttggttcgggccgaaaatcattgggaaatggtgcagcgggaaacttttgctcccatcctttaccttattccctttaaaaccctggaggaggcgattgcactgaacaatgccgtacctcaagggttttcctcgtcattatttaccactcatctccagcatgccgaacgatttctctcccactggggcagtgattgtggtatcgccaacattaatatgggtacgtcaggggctgagatcggcggggcttttgggggtgagaaggaaactggaggaggtcgagaagcgggctcggatgcttggaaaaactatatgcggcggcaaaccaataccatcaattggggcacggaattgcccctggctcaaggaattcgcttcgagctggagggggagagcccaccagagagatga

SEQ ID NO: 79 - protein, 524 aa, Aminomonas paucivorans
MGTTTMTSVLRDSILAEVGLEEVPSGATLGGSGGWLDVHGPELVSRSPTDGQEIARIRQADGSDLARVLDGAREGFRTWSAMPAPRRGEVVRRIGTQLRRYKEPLARLITLEMGKILPEARGEVQEMIDIADFAVGLSRQLYGMTTHSERPGHRLYEQWHSLGPVGVVTAFNFPSAVWSWNAMLAGVCGDTVVWKPSSQTPLVTLAVQRILEPVLDEFDLRGVFTAVVGPGRSVGEELLRSPHIPLLSVTGSGSVGAHVAQVVGARMGRTILELGGNNAVIVREDACLDDALQAVFFGAVGTAGQRCTSTRRVILHPRIAKPFLDRLCSAYRHLKVDHPLWTDPHVGPVTTPEAMEDMESAIREARDAGGTLLAGGDRVPVEGAPEGYYIRPAIMRAENHWPVVQRETFAPLLYVMEAESMEEALALQNGVPQGLSSSCFTNSLRDAERFLSVRGSDCGIANINLGTSGAEIGGAFGGEKETGGGRESGSDAWKAYMRRQTVTIHWEDGLELAQGVVFKLPGEA

SEQ ID NO: 80 - DNA, 1575 bp, Aminomonas paucivorans
atgggtaccacgaccatgacgagcgtgctgagggattccatccttgcggaggtggggttggaagaggttccttccggggcgaccttgggcgggagcggcgggtggctggacgtgcacgggccggagctggtctcccgctccccaacggatggacaggagatcgctcggatccggcaggccgacggatccgacctggctcgggtcctcgacggggccagggaagggttcaggacctggtccgccatgcccgccccccggcgcggggaggtggtgcggcgcatcgggacgcagctacgccggtacaaggagcccctggcccggctcatcaccctggagatggggaagatcctcccggaggctcggggagaggtccaggagatgatcgacatcgccgacttcgccgtgggactcagcaggcagctgtacggcatgaccacccacagcgagcggcccggtcatcggttgtacgaacagtggcactccctggggccggtgggggtcgtcacggccttcaacttccccagcgcggtctggagttggaacgccatgctcgcgggggtctgcggggacacggttgtctggaagcccagttcccagacccccctggtgaccttggcggtgcagcggatcctggagcccgtcctggacgagttcgaccttcggggtgtgtttaccgccgtcgtcggtccggggcgcagcgtgggagaggagctgcttcggtcgccccacatccccctgctcagcgtcaccggcagcggttccgtgggagcccacgtggctcaggtggtgggggcgcgcatgggacgcaccatcctggagctggggggcaacaacgccgtgatcgtccgggaggacgcctgcctggacgacgcgcttcaggcggtcttcttcggggcggtggggacggcgggacagcgctgcaccagtacccgcagggtgatcctgcaccctcggatcgccaagccgttcctggaccgtctctgctcggcctaccgccatctgaaggtcgaccatcccctgtggacggacccccacgtgggtcccgtgaccacccccgaggccatggaggacatggagtcggccatccgggaggcccgagacgccggggggaccctgctggcggggggagaccgcgttcctgtggagggagcgccggagggatactacatccgcccggccatcatgagggcggagaaccactggccggtggtgcagagggagaccttcgctcccctcctctacgtcatggaggcggagagcatggaggaggccctggctctgcagaacggcgtgcctcagggcttgagctcctcttgcttcaccaattccctgcgggacgcggagcggttcctctcggtccgggggtcggactgcggcatcgccaacatcaacctggggacgtcgggagctgaaatcggcggggccttcgggggcgaaaaggagaccggcggcggccgggaatcggggtccgacgcctggaaagcctacatgcgccgccagaccgtcaccatccactgggaggatggtttggaactggcacagggcgtggtcttcaagctgcccggggaggcatga

SEQ ID NO: 81 - protein, 514 aa, Solibacter usitatus
MPSANLTTHAHSVPEILEKLHVEPVNSGACFGDWIAEPSGGELVSSSPATGEALARVRTAGPGDYETVMEFATAAFLEWRMLPAPKRGEIVREIGNELRAHKQHLGALVSLEMGKILPEGLGEVQEMIDICDFAVGLSRQLYGLTMHSERPGHRMYEQWHPLGVVGVISAFNFPVAVWAWNAMIAAVCGDCVLWRPSSETPLLAIAVQKIVNRVFDRHGLKGIFNLVIGPSNPIGEILVKDPRIPLISFTGSTEVGRRVAEVIAHRLGRSILELGGNNAIIVMDDANADLVLRAVLFGCVGTAGQRCTTTRRLFLQRGIAPRIREALTRAYGQVRIGHPLDEQTIMGPLVNRRAVDDMLDGLRRIREQGGEILYGGEALEGCYVQPTLVRARPDMPILKEEIFAPILYLIEFDHLDEAIHWHNDVPQGLSSAMFTTNVISSETFLSHRGSDCGIANINIGTSGAEIGGAFGGEKETGGGRESGSDAWKAYMRRQTNTINWSTQLPLAQGIEFKL

SEQ ID NO: 82 - DNA, 1545 bp, Solibacter usitatus
atgccctccgcgaacctgaccacccacgcgcactcagttcccgagattctcgaaaaacttcacgtcgagcctgtaaattccggcgcttgtttcggcgactggatcgccgaaccgtccggcggggaactcgtttccagcagtcccgcgaccggcgaggcgctggcgcgggtgcgcacggccggcccaggcgattacgagaccgtgatggagttcgccaccgcggccttcctggagtggcgcatgcttcctgcgccgaagcgcggcgaaatcgtccgcgaaatcggcaatgagctgcgcgctcacaagcagcacctgggagcgctggtttcgctcgagatgggaaagattctgcccgagggcctgggcgaagtgcaggagatgatcgacatctgcgatttcgcggtcggcctctcgcgccaattgtacgggctcaccatgcacagcgagcgcccgggccaccgcatgtacgaacagtggcatccgctgggcgtggtgggcgtgatcagcgcgttcaatttcccggtggcggtgtgggcgtggaacgcaatgatcgccgccgtgtgcggcgattgcgtactgtggcggccctcgtcggaaaccccgctgctcgcgatcgcggtgcagaagatcgtgaaccgcgtgttcgaccggcacgggctgaagggcatcttcaacctcgtgatcgggccgagcaacccgatcggcgagatactggtgaaggacccgcgcattccgttgatcagcttcacgggttccaccgaggtcggccgccgcgttgccgaggtgatcgcgcaccggcttggccgctcaatcctggagctcggcggcaataacgcgatcatcgtgatggacgacgccaatgccgacctcgtgctgcgcgccgtgctcttcgggtgcgtcggcacggccggccagcgctgcaccaccacgcggcgcctgttcctgcagcgaggcattgcaccgcgtatcagggaggcgctgacgcgcgcgtatgggcaggtgcggatcggccacccgctcgacgaacaaaccatcatgggtccgctggtcaatcgccgggcggtggacgatatgctcgatggtcttcggcgcatccgcgagcagggcggcgagatcctttacggcggcgaagctctggagggctgctacgtgcagccgacgctggtccgcgcgcggccggacatgccgatcctcaaggaagaaatcttcgcgccgattctctacctgatcgagttcgaccacctggatgaggcgatccactggcacaacgatgttccgcagggcctttcgtcggcaatgttcacgacgaacgtgatctcgtcggagacgtttttgagtcatcgcggcagcgattgcggaatcgccaatatcaatatcggcaccagcggggctgagatcggcggcgcgttcggcggagagaaggaaaccggcgggggacgcgaatcggggagtgacgcgtggaaggcctacatgcgaaggcagaccaacacgatcaactggtccacacaattgccattagctcaggggattgaatttaagctgtag

(19) United States
(12) Patent Application Publication
(10) Pub. No.: US 2014/0068810 A1
(43) Pub. Date: Mar. 6, 2014

(54) USE OF ALDH7 FOR IMPROVED STRESS TOLERANCE

(71) Applicant: PIONEER HI BRED INTERNATIONAL INC, Johnston, IA (US)

(72) Inventor: Norbert Brugiere, Johnston, IA (US)

(73) Assignee: PIONEER HI BRED INTERNATIONAL INC, Johnston, IA (US)

(21) Appl. No.: 14/013,089

(22) Filed: Aug. 29, 2013

Related U.S. Application Data
(60) Provisional application No. 61/694,379, filed on Aug. 29, 2012.
(60) Provisional application No. 61/783,741, filed on Mar. 14, 2013.

(51) Int. Cl. C12N 15/82

(52) U.S. Cl. CPC C12N 15/8273
USPC 800/278; 800/298; 800/320.1

(57) ABSTRACT

The present invention relates to the field of plant molecular biology, more particularly to the regulation of genes that increase drought tolerance and yield. Provided herein are methods finding use in agriculture for increasing drought tolerance in dicot and monocot plants. Methods comprise introducing into a plant cell a polynucleotide that encodes an ALDH7 polypeptide operably linked to a promoter that drives expression in a plant. Also provided are transformed plants, plant tissues, plant cells, and seeds thereof.

### CROSS REFERENCE

This utility application claims the benefit of U.S. Provisional Application Ser. No. 61/694,379, filed Aug. 29, 2012 and U.S. Provisional Application Ser. No. 61/783,741 filed Mar. 14, 2013, both of which are incorporated herein by reference.

### FIELD OF THE INVENTION

The present invention relates to the field of plant molecular biology, more particularly to the regulation of genes that increase drought tolerance and yield.

### BACKGROUND OF THE INVENTION

Insufficient water for optimum growth and development of crop plants is a major obstacle to consistent or increased food production worldwide. Population growth, climate change, irrigation-induced soil salinity, and loss of productive agricultural land to development are among the factors contributing to a need for crop plants which can tolerate drought. Drought stress often results in reduced yield.

Plants are restricted to their habitats and must adjust to the prevailing environmental conditions of their surroundings. To cope with abiotic stressors in their habitats, higher plants use a variety of adaptations and plasticity with respect to gene regulation, morphogenesis, and metabolism. Adaptation and defense strategies may involve the activation of genes encoding proteins important in acclimation or defense against different stressors, including drought. Understanding and leveraging the mechanisms of abiotic stress tolerance will have a significant impact on crop productivity. Methods are needed to enhance tolerance to drought and other abiotic stresses and to reduce yield loss in drought conditions.

Aldehyde molecules are produced as intermediates in numerous metabolic pathways (Kirch, et al., 2004). While a certain level of aldehydes may be useful in signaling (Weber, et al., 2004), excessive accumulation of aldehydes may lead to production of reactive oxygen species (ROS), resulting in oxidative stress (Lamb & Dixon, 1997; Bolwell. 1999). Methods to manipulate aldehyde accumulation are of interest for improving plant abiotic stress tolerance.

### SUMMARY OF THE INVENTION

Methods are provided for increasing drought tolerance in plants. More particularly, the methods of the disclosure find use in agriculture for increasing drought tolerance in dicot and monocot plants. Certain embodiments comprise introducing into a plant cell a polynucleotide that encodes an ALDH7 polypeptide operably linked to a promoter that drives expression in a plant. Also provided are transformed plants, plant tissues, plant cells, and seeds thereof.

The following embodiments are among those encompassed by the present invention.
1. A method for increasing drought tolerance in a plant, said method comprising introducing into said plant a polynucleotide construct comprising a nucleotide sequence encoding a polypeptide which directs, expands, amplifies, or accelerates the degradation of aldehydes or results in the reduced accumulation of aldehydes in the plant, and expressing said polynucleotide in said plant, wherein drought tolerance of said plant is increased relative to a control plant.
2. The method of embodiment 1 wherein the reduced accumulation of aldehyde occurs in leaf and/or root tissue.
3. The method of embodiment 1 wherein said polynucleotide encodes a polypeptide having at least 95% sequence identity to the full length of SEQ ID NO: 2, wherein said nucleotide sequence is operably linked to a heterologous promoter selected from the group consisting of a weak constitutive promoter, an organ-preferred or tissue-preferred promoter, a stress-inducible promoter, a chemical-inducible promoter, a light-responsive promoter, and a diurnally-regulated promoter.
4. The method of embodiment 3, wherein said weak constitutive promoter is a GOS2 promoter or rice actin promoter.
5. The method of embodiment 3, wherein said tissue-preferred promoter is a leaf-preferred promoter, a root-preferred promoter, a vasculature-specific promoter or a promoter which does not drive expression in developing or mature ears.
6. The method of embodiment 3, wherein said stress-inducible promoter is a Rab17 promoter or an Rd29a promoter.
7. The method of embodiment 3, wherein said light-responsive promoter is an rbcS (ribulose-1,5-bisphosphate carboxylase) promoter, a Cab (chlorophyll a/b-binding) promoter or a phosphoenol-pyruvate carboxylase (PEPc) promoter.
8. The method of any of embodiments 1-7, wherein said nucleotide sequence encodes a polypeptide having at least 95% sequence identity to the full length of SEQ ID NO: 2,
9. A method for increasing yield of a seed crop plant exposed to drought stress, said method comprising increasing expression in said plant of a polynucleotide operably linked to a heterologous promoter, wherein said expression results in reduced accumulation of aldehydes in the plant.
10. The method of embodiment 1. wherein the polynucleotide encodes a polypeptide selected from the group consisting of:
   a) SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
   b) A polypeptide at least 95% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74; and
   c) A polypeptide at least 90% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74.
11. The method of embodiment 9, wherein lipid peroxidation under drought stress is reduced.
12. The method of any of embodiments 9-11, wherein accumulation of one or more drought-induced aldeyhydes under drought stress is reduced.
13. The method of embodiment 12, wherein accumulation of malondialdehyde is reduced.
14. The method of any of embodiments 9 through 13, wherein increased expression occurs primarily during daytime hours.
15. The method of any of embodiments 9 through 13, wherein said seed crop plant is selected from the group consisting of a grain plant, an oil-seed plant, and a leguminous plant.
16. The method of embodiment 15, wherein said grain plant is maize or wheat.
17. The method of embodiment 15, wherein said oil-seed plant is a Brassica plant.
18. The method of any one of embodiments 9-16, wherein said promoter is an early kernel/embryo promoter.
19. The method of any one of embodiments 9-18, wherein a nucleotide sequence encoding said polypeptide is introduced into said plant by breeding or by transformation.
20. A plant comprising a polynucleotide construct comprising a nucleotide sequence operably linked to a heterologous promoter, wherein said nucleotide sequence is selected from the group consisting of:
   a. SEQ ID NO: 1, 61, 63, 65, 67, 69, 71, 73, 75 and 76;
   b. A polynucleotide at least 95% identical to any of SEQ ID NO: 1, 61, 63, 65, 67, 69, 71, 73, 75 and 76;
   c. A polynucleotide at least 90% identical to any of SEQ ID NO: 1, 61, 63, 65, 67, 69, 71, 73, 75 and 76; and
   d. Operable fragments and variants of any of SEQ ID NO: 1, 61, 63, 65, 67, 69, 71, 73, 75 and 76.
21. The plant of embodiment 20, wherein heterologous promoter is selected from the group consisting of a constitutive promoter, weak constitutive promoter, an organ-preferred or tissue-preferred promoter, a stress-inducible promoter, a chemical-inducible promoter, a light-responsive promoter, and a diurnally-regulated promoter.
22. The plant of embodiment 20, wherein said plant is a seed crop plant.
23. The plant of embodiment 20, wherein said plant exhibits an increase in drought tolerance relative to a control plant.
24. A transformed seed of the plant of any one of embodiments 20-23.
25. The plant of embodiment 20, wherein said plant is maize and wherein the grain yield of a plurality of said plants exceeds 150 bushels per acre.
26. The plant of embodiment 25, wherein said grain yield occurs under drought conditions.
27. The plant of embodiment 20, wherein said plant is maize and wherein the grain yield of a plurality of said plants is at least 3% greater than the yield of a plurality of control plants.
28. The plant of embodiment 27, wherein said yield increase occurs under drought conditions.
29. The plant of embodiment 20, further comprising a heterologous polynucleotide encoding an abscisic acid (ABA)-associated polypeptide.

### BRIEF DESCRIPTION OF THE DRAWINGS

FIG. 1 shows the diurnal expression pattern of the native maize ALDH7 gene.

FIG. 2 shows drought-induced expression of endogenous ZmALDH7 in maize leaf at two different developmental stages (A and B), in root (C) and immature ear (D) as measured by massively parallel signature sequencing (Illumina, Inc.) and drought induction of ZM-ALDH7 expression n in shoots of B73 seedlings measured by Northern blot analysis (E).

FIG. 3 provides mass spectrometry data for two endogenous ZmALDH7 peptides indicating increased levels of the native protein under drought conditions (“water stressed”) compared to well-watered conditions (“watered”). Plants at V6-V7 stage were subjected to 30 hours of drought stress. ZmALDH7 increase in leaves was greater than 3-fold.

FIG. 4 is a Northern blot of maize T0 leaf tissue showing expression of transgenic ZmALDH7 driven by the Ubiquitin promoter, compared to expression in control tissue, for 16 transgenic events. Fourteen of the sixteen events show consistent transgene expression at a much higher level than expression of endogenous ZmALDH7 in control plants.

FIG. 5 shows ZmALDH7 protein levels detected by mass-spectrometry in control and transgenic plants over-expressing ZmALDH7 under the control of the Ubiquitin promoter. A 9-fold increase in levels of ZmALDH7 protein was identified in leaves of transgenic plants compared to controls (right panel). Under water-stressed conditions a 5 fold increase was detected (left panel).

FIG. 6 provides an alignment of maize (SEQ ID NO: 2) and rice (SEQ ID NO: 44; GenBank AF323586) ALDH7 polypeptides. FIG. 6A shows alignment of positions 1 through 300. FIG. 6B shows alignment of positions 301 through 509.

FIG. 7 is a dendogram of maize, rice and Arabidopsis ALDH genes. Distinct diurnal cycling expression patterns have been observed in Family 11, Family 3, Family 5, Family 7, Family 10 and Family 2. There are 23 maize ALDH genes in ten families. In all three species, the ALDH7 family is represented by a single gene. The dendogram is presented on two pages, FIGS. 7A and 7B, in order to meet font size and reproducibility requirements.

FIG. 8 shows relative expression levels of endogenous ALDH7 in various tissues of B73 maize determined by Northern blot using a specific 3′ UTR probe. “DAP”=days after pollination.

FIG. 9A shows that endogenous ZmALDH7 expression determined by MPSS. (Brenner, et al., (2000) Nature Biotechnol. 18:630-634) is induced by treatment of leaf material by abscisic acid (ABA). FIG. 9B shows induction of ZM-ALDH7 expression determined by Northern blot analysis after floating discs of maize B73 leaf tissue on solution containing 0, 0.5, 5 or 10 μM ABA for 24 hours under constant light.

FIG. 10 shows that T2 Arabidopsis seeds transgenic for a construct comprising the 35S promoter driving ZmALDH7 (events E1, E3, E4, E7) have enhanced Paraquat tolerance, relative to wild-type (WT) seeds, as reflected in germination. ZmALDH7 protein levels in corresponding T1 plants are also shown. The peptide used for mass-spectrometry determinations of ZmALDH7 protein levels is different than the peptide present in Arabidopsis ALDH7B4 and is therefore specific to ZmALDH7 (alignment).

FIG. 11 shows that under a triple stress test (drought, heat and excess light), transgenic Arabidopsis plants for a construct comprising the 35S promoter driving ZmALDH7 had more rapid growth (left panel) and greater maximum growth (right panel), than wild-type plants.

FIG. 12 shows that under salt-stress conditions, hybrid maize seedling roots comprising a Ubi:ZmALDH7 construct accumulated less malondialdehyde (MDA) than did control seedling roots.

FIG. 13 provides a model of the ZmALDH7 protein and structural similarities to antiquitin from seabream fish (Tang, et al., (2008) Febs Letters 582:3090-3096).

FIG. 14 shows the size of the purified his-tagged recombinant ZmALDH7 protein (His-ALDH7) and recombinant protein with His tag removed using thrombin cleavage (ALDH7) using SDS-PAGE. Right panel shows comparison of commercial yeast ALDH (Sigma) compared with recombinant ZmALDH7.

FIG. 15 shows a time course of recombinant ZmALDH7 activity at pH 8.0. Reaction conditions: 100 mM Tris, 100 mM KCl, 10 mM 2-Mercaptoethanol, 0.667 mM β-Nicotinamide Adenine Dinucleotide, Oxidized Form (β-NAD), 1.67 mM Acetaldehyde, in 0.6 ml volume at room temperature.

FIG. 16 indicates relative activity of ZmALDH7 at various pH levels.

FIG. 17 indicates relative activity of ZmALDH7 on various substrates at pH 7.0.

FIG. 18 compares time courses of maize and yeast ALDH7 activity at pH 7.0. Reaction conditions: 100 mM Tris, 100 mM KCl, 10 mM 2-Mercaptoethanol, 1 mM β-Nicotinamide Adenine Dinucleotide, Oxidized Form (β-NAD), 2.5 mM Propionaldehyde, 1 μg protein, in 0.6 ml volume at room temperature.

FIG. 19 shows that Arabidopsis ALDH7B4 RNAi events with down-regulated ALDH7B4 expression (in bold top panel, Northern) are hypersensitive to high lysine levels (bottom panel)

FIG. 20 shows kinetic properties of recombinant ZmALDH7 with different aldehyde substrates, measured in vivo.

FIG. 21A shows cytoplasmic localization of a ZmALDH7:AcGFP1 translational fusion in maize leaf epidermis pavement cell; B shows cell wall autofluorescence.

BRIEF DESCRIPTION OF THE SEQUENCES

| SEQ ID NO: | Species | Name |
|---|---|---|
| 1 | Zm | ALDH7-CDS |
| 2 | Zm | ALDH7 |
| 3 | Zm | ALDH12 |
| 4 | Zm | ALDH3B1 |
| 5 | Zm | ALDH3E2 |
| 6 | Zm | ALDH3H1 |
| 7 | Zm | ALDH3H2 |
| 8 | Zm | ALDH3I1 |
| 9 | Zm | ALDH3I2 |
| 10 | Zm | ALDH5F1 |
| 11 | Zm | ALDHH2C4 |
| 12 | Zm | ALDH10A8 |
| 13 | Zm | ALDH10A5 |
| 14 | Zm | ALDH10A9 |
| 15 | Zm | ALDH2C1 |
| 16 | Zm | ALDH2B2 |
| 17 | Zm | ALDH2B5-2 |
| 18 | Zm | ALDH2B5-2 |
| 19 | Zm | ALDH11A3 |
| 20 | Zm | ALDH22A1 |
| 21 | Zm | ALDH6B2 |
| 22 | Zm | ALDH2B1 |
| 23 | Zm | ALDH18B1 |
| 24 | Zm | ALDH18B2 |
| 25 | At | ALDH12A1 |
| 26 | At | ALDH3F1 |
| 27 | At | ALDH3I1 |
| 28 | At | ALDH3H1 |
| 29 | At | ALDH22A1 |
| 30 | At | ALDH11A3 |
| 31 | At | ALDH6B2 |
| 32 | At | ALDH7B4 |
| 33 | At | ALDH5F1 |
| 34 | At | ALDH2C4 |
| 35 | At | ALDH2B4 |
| 36 | At | ALDH2B7 |
| 37 | At | ALDH10A8 |
| 38 | At | ALDH10A9 |
| 39 | Os | ALDH3E1 |
| 40 | Os | 04g45730.1 |
| 41 | Os | ALDH22A1 |
| 42 | Os | ALDH11A3 |
| 43 | Os | ALDH6B2 |
| 44 | Os | ALDH7B6 |
| 45 | Os | ALDH2C4 |
| 46 | Os | ALDH2C1 |
| 47 | Os | ALDH2B5 |
| 48 | Os | ALDH2B1 |
| 49 | Os | ALDH10A5 |
| 50 | Os | ALDH10A8 |
| 51 | Os | ALDH2B2 |
| 52 | OS | ALDH3B1 |
| 53 | Os | ALDH3E2 |
| 54 | Os | ALDH3H1 |
| 55 | Os | ALDH3H2 |
| 56 | Os | ALDH5F1 |
| 57 | Os | ALDH12A1 |
| 58 | Os | ALDH12B1 |
| 59 | Os | ALDH18B1 |
| 60 | Os | ALDH18B2 |
| 61 | Ec | ALDH7 |
| 62 | Ec | ALDH7aa |
| 63 | Th | ALDH7 |
| 64 | Th | ALDH7aa |
| 65 | Ps | ALDH7 |
| 66 | Ps | ALDH7aa |
| 67 | Tr | ALDH7 |
| 68 | Tr | ALDH7aa |
| 69 | Si | ALDH7 |
| 70 | Si | ALDH7aa |
| 71 | Gm | ALDH7 |
| 72 | Gm | ALDH7aa |
| 73 | Ps | ALDH7-2 |
| 74 | Ps | ALDH7aa-2 |
| 75 | At | ALDHI7B4-nt |
| 76 | At | ALDH3I1 |

### DETAILED DESCRIPTION OF THE INVENTION

Methods are provided for increasing stress tolerance, particularly abiotic stress tolerance, in plants. These methods find use, for example, in increasing tolerance to drought stress and maintaining or increasing yield during drought conditions, particularly in agricultural plants. The methods involve genetically manipulating a plant to alter the expression of ZmALDH7. In some embodiments, crop yield is maintained or increased by ameliorating the detrimental effects of drought stress on membrane integrity in agriculturally important plants.

The methods comprise stably incorporating into the genome of a plant a DNA construct comprising a nucleotide sequence which encodes a maize ALDH7 polypeptide, operably linked to a promoter that drives expression in a plant. A maize ALDH7 polynucleotide is disclosed herein as SEQ ID NO: 1, and its encoded polypeptides is disclosed herein as SEQ ID NO: 2. Other maize ALDH genes are also provided.

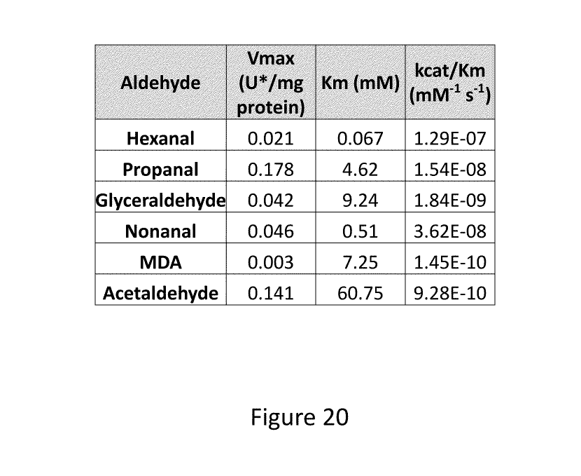

Without being bound to any theory, ALDH7 may be part of a coordinated osmotic/oxidative stress response that may involve lysine catabolism. In human, mutations in antiquitin (ALDH7A1) were identified as the cause of pyridoxine dependent epilepsy. ATQ functions as an aldehyde dehydrogenase (ALDH7A1) in the lysine degradation pathway. Mutations result in accumulation of alpha-aminoadipic semialdehyde (AASA), piperideine-6-carboxylate (P6C) and pipecolic acid. Arabidopsis seedlings in which ALDH7 expression is down-regulated show hypersensitivity to lysine (FIG. 20) indicating that a link between the lysine pathway and aldehyde detoxification also exists in plants.

The disclosed ZmALDH7 protein (SEQ ID NO: 2) shows the following level of identity to Arabidopsis (At), soy (Gm), and rice (Os) ALDH7 proteins:

TABLE 1
Identity and Similarity of ALDH7 orthologues.

| | Gm-ALDH7 | Os-ALDH7 | Zm-ALDH7 |
|---|---|---|---|
| | SEQ ID NO: 72 | SEQ ID NO: 44 | SEQ ID NO: 2 |
| % Identity | | | |
| AtALDH7B4 | 76.8 | 78.4 | 78.0 |
| SEQ ID NO: 32 | | | |
| Gm-ALDH7 | | 78.3 | 77.9 |
| Os-ALDH7 | | | 92.7 |
| % Similarity | | | |
| AtALDH7B4 | 86.7 | 89.0 | 89.0 |
| Gm-ALDH7 | | 85.4 | 85.5 |
| Os-ALDH7 | | | 97.6 |

In one aspect, methods are provided for increasing abiotic stress tolerance, such as drought tolerance, in a plant. In some embodiments, the methods can comprise introducing into a plant a polynucleotide construct comprising a nucleotide sequence encoding a polypeptide having at least about 95% amino acid sequence identity to SEQ ID NO: 2, or a variant or fragment thereof, operably linked to a heterologous promoter that is functional in a plant cell. In certain embodiments, when a nucleotide sequence provided herein is expressed in the plant, drought tolerance of the plant is increased relative to a control plant. In some cases, the nucleotide sequence encodes a polypeptide having at least about 70%, about 75%, about 80%, about 85%, about 90%, about 95%, about 97%, about 99% or about 100% amino acid sequence identity to SEQ ID NO: 2, or a variant or fragment thereof. In some cases, the nucleotide sequence encodes SEQ ID NO: 2.

ZmALDH7 polypeptides disclosed herein can be altered in various ways including amino acid substitutions, deletions, truncations and insertions. Methods for such manipulations are generally known in the art. For example, sequence variants of the ZmALDH7 polypeptide of SEQ ID NO: 2 can be prepared by mutations in the DNA encoding it. Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. A mutagenic and recombinogenic procedure such as DNA shuffling can be employed to alter the ZmALDH7 polypeptide disclosed herein. Thus, the genes and nucleotide sequences of the invention involve both the naturally occurring-sequences and mutant forms. Likewise, the proteins of the invention encompass naturally occurring polypeptides as well as variations and modified forms thereof. Such variants will continue to possess the desired functional activity. In that regard, mutations that will be made in the DNA encoding the variant must not place the sequence out of reading frame and preferably will not create complementary regions that could produce secondary mRNA structure. See, EP Patent Number 0075444 B1.

Accordingly, the present disclosure encompasses the maize ZmALDH7 polypeptide as well as active variants and fragments thereof. That is, it is recognized that variants and fragments of the proteins may be produced that retain the ability to improve stress tolerance of the plant. Such ability may reflect a role in detoxification of lipid-peroxidation-derived reactive aldehydes. Such variants and fragments include truncated sequences as well as N-terminal, C-terminal, and internally-deleted amino acid sequences of the proteins. By “fragment” is intended a portion of the polynucleotide or a portion of the amino acid sequence and hence of the protein encoded thereby. Fragments of a polynucleotide may encode protein fragments that retain biological activity and hence retain the ability to improve stress tolerance of a plant. Alternatively, fragments of a polynucleotide which are useful as hybridization probes generally do not encode fragment proteins retaining biological activity. Thus, fragments of a nucleotide sequence may range from at least about 20 nucleotides to about 50 nucleotides, about 100 nucleotides, and up to the full-length polynucleotide encoding a maize ALDH7 protein.

A fragment of a polynucleotide that encodes a biologically active portion of a claimed ZmALDH7 protein will encode at least about 15, about 25, about 30, about 50, about 100 or about 150 contiguous amino acids or up to the total number of amino acids present in a full-length ZmALDH7 protein of the disclosure (i.e., 509 amino acids for SEQ ID NO: 2). Fragments of a polynucleotide which are useful as hybridization probes or PCR primers generally need not encode a biologically active portion of ZmALDH7 protein. Thus, a fragment of a polynucleotide may encode a biologically active portion of a ZmALDH7 protein or it may be a fragment that can be used as a hybridization probe or PCR primer using methods disclosed below. A biologically active portion of a ZmALDH7 protein can be prepared by isolating a portion of a ZmALDH7 polynucleotide, expressing the encoded portion of the ZmALDH7 protein (e.g., by recombinant expression in vitro), and assessing the activity of the encoded portion of the ZmALDH7 protein. Polynucleotides that are fragments of a ZmALDH7 nucleotide sequence comprise at least about 75, about 100, about 150, about 200, about 250, about 300, about 350, about 400, about 450 or about 470 contiguous nucleotides, or up to the number of nucleotides present in a full-length ZmALDH7 polynucleotide disclosed herein (i.e. 1527 for SEQ ID NO: 1).

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a ZmALDH7 polypeptide disclosed herein. Variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a ZmALDH7 protein disclosed. Generally, variants of a particular polynucleotide will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein.

Variants of a particular reference polynucleotide disclosed can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 2 is disclosed. Percent sequence identity between any two polypeptides can be calculated using sequence alignment programs and parameters described elsewhere herein. Where any given pair of polynucleotides is evaluated by comparison of the percent sequence identity shared by the two polypeptides they encode, the percent sequence identity between the two encoded polypeptides is at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention may be biologically active; that is, they continue to possess the desired biological activity of the native protein, that is, the ability to increase abiotic stress tolerance, perhaps by improving membrane stability by detoxifying reactive oxygen species. Such variants may result from, for example, genetic polymorphism or from human manipulation. Biologically active variants of a native ZmALDH7 protein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs and parameters described elsewhere herein. A biologically active variant of a reference protein may differ from that protein by as few as 1-15 amino acid residues, as few as 1-10, such as 6-10, as few as 5, as few as 4, 3, 2 or even 1 amino acid residue.

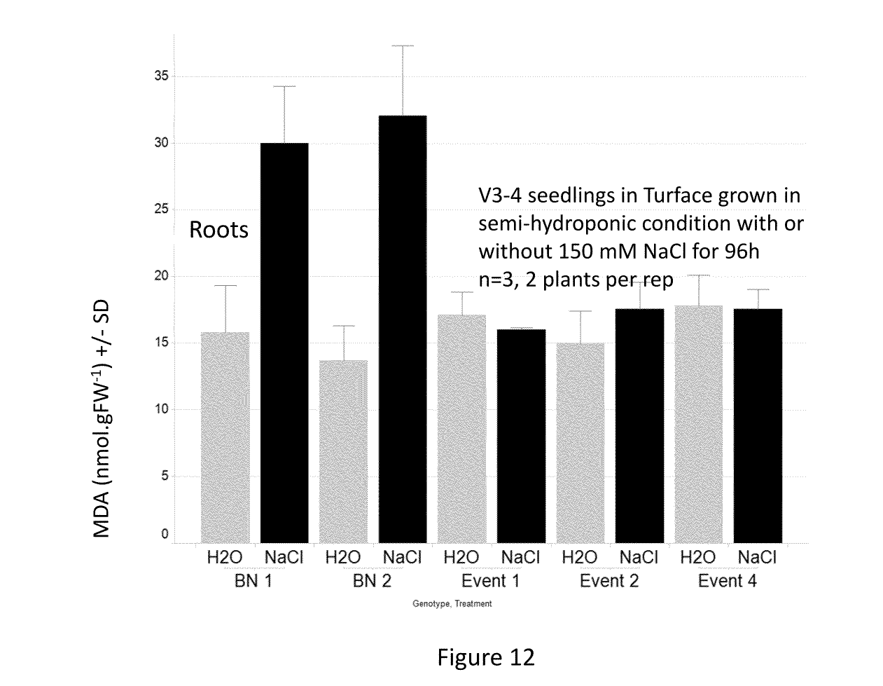

In certain embodiments, disclosed proteins may be altered in various ways including amino acid substitutions, deletions, truncations, and insertions. Methods for such manipulations are generally known in the art. For example, amino acid sequence variants and fragments of the ZmALDH7 protein can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions and substitutions of the protein sequences encompassed herein are not expected to produce radical changes in the characteristics of the protein. When it is difficult, however, to predict the exact effect of a substitution, deletion, or insertion in advance of making such modifications, one skilled in the art will appreciate that the effect will be evaluated by routine screening assays. That is, changes in abiotic stress tolerance can be evaluated by standard methods known to those of ordinary skill in the art. Means for measuring lipid hydroperoxides are commercially available (see, e.g., IBL International). FIG. 12, for example, provides data for reduced levels of malondialdehyde in roots after salt stress.

The following terms are used to describe the sequence relationships between two or more polynucleotides or polypeptides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244; Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990), supra. BLAST nucleotide searches can be performed with the BLASTN program, score=100, wordlength=12, to obtain nucleotide sequences homologous to a nucleotide sequence encoding a ZmALDH7 protein. BLAST protein searches can be performed with the BLASTX program, score=50, wordlength=3, to obtain amino acid sequences homologous to a ZmALDH7 protein or polypeptide. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997), supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2 and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As described herein, a nucleotide sequence encoding a ZmALDH7 polypeptide, variant, or fragment thereof as provided herein is operably linked to a promoter that drives expression of the sequence in a plant. Any one of a variety of promoters can be used with a ZmALDH7 sequence, depending on the desired timing and location of expression. In some cases, the promoter is a constitutive promoter, a tissue-preferred promoter, a chemical-inducible promoter, a stress-inducible promoter, a light-responsive promoter, or a diurnally-regulated promoter. For example, constitutive promoters can be used to drive expression of a nucleotide sequence of interest. The most common promoters used for constitutive overexpression are derived from plant virus sources, such as the cauliflower mosaic virus (CaMV) 35S promoter (Odell, et al., (1985) Nature 313:810-812). The CaMV 35S promoter delivers high expression in virtually all regions of transgenic monocot and dicot plants. Constitutive promoters also can include, for example, the core promoter of the Rsyn7 promoter and other constitutive promoters disclosed in WO 1999/43838 and U.S. Pat. No. 6,072,050; rice actin (McElroy, et al., (1990) Plant Cell 2:163-171); ubiquitin (Christensen, et all, (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611.

Transgene expression can be beneficially adjusted by using a promoter suitable for the plant's background and/or for the type of transgene. Where low level expression is desired, weak promoters can be used. It is recognized that weak constitutive, weak inducible, or weak tissue-preferred promoters can be used. Generally, by “weak promoter” is intended a promoter that drives expression of a coding sequence at a low level. By low level is intended at levels of about 1/1000 transcripts to about 1/100,000 transcripts to about 1/500,000 transcripts. An example of a weak constitutive promoter is the GOS2 promoter; see, U.S. Pat. No. 6,504,083.

In some embodiments, the ZmALDH7 sequences can be utilized with tissue-preferred or developmental-preferred promoters to drive expression of the sequence of interest in a tissue-preferred or a developmentally-preferred manner. For example, tissue-preferred promoters such as leaf-preferred promoter or root-preferred promoters can be used. While the claims are not bound by any particular theory or mechanism of action, it is believed that expression of ZmALDH7 in a diurnal manner which is counter to native expression would promote drought tolerance in the plant, as drought stress typically occurs during the day due to lower humidity and increased evapotranspiration. Exemplary regulatory elements having diurnal expression patterns are disclosed for example in US Patent Application Publication Number 2011/0167517, which is hereby incorporated by reference.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590. Increased expression ALDH7 in leaves may be of particular interest. Leaf expression of the endogenous ALDH7 gene is not observed in maize.

Root-preferred promoters are also known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. Leach and Aoyagi, (1991) describe their analysis of the promoters of the highly expressed roIC and rolD root-inducing genes of Agrobacterium rhizogenes (see, Plant Science (Limerick) 79(1):69-76). Teen, et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene, fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772) and rolB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179. Other root-preferred promoters include Zm-NAS2 promoter (U.S. Pat. No. 7,960,613), Zm-Cyclo1 promoter (U.S. Pat. No. 7,268,226), Zm-Metallothionein promoters (U.S. Pat. Nos. 6,774,282; 7,214,854 and 7,214,855 (also known as RootMET2)), Zm-MSY promoter (US Patent Application Publication Number 2009/0077691) or MsZRP promoter (U.S. Pat. No. 5,633,363).

Other promoters may be utilized to drive expression of a maize ZmALDH7 polynucleotide, such as the promoter of the maize KZM2 gene (see, Buchsenschutz, et al., (2005) Planta 222:968-976 and NCBI AY919830) or a green-tissue-preferred promoter (US Patent Application Publication Number 2011/0209242).

Constructs may also include one or more of the CaMV35S enhancer, Odell, et al., (1988) Plant Mol. Biol. 10:263-272, the ADH1 INTRON1 (Callis, et al., (1987) Genes and Dev. 1:1183-1200), the UBI1ZM INTRON(PHI) as an enhancer, and PINII terminator.

In some embodiments, the ZmALDH7 sequences can be utilized with stress-inducible promoters to drive expression of the sequence of interest in a stress-regulated manner. A stress-inducible promoter can be, for example, a rabl7 promoter (Vilardell, et al., (1991) Plant Molecular Biology 17(5):985-993; Busk, et al., (1997) Plant J 11(6):1285-1295) or rd29a promoter (Yamaguchi-Shinozaki and Shinozaki, (1993) Mol. Gen. Genet. 236:331-340; Yamaguchi-Shinozaki and Shinozaki, (1994) Plant Cell 6:251-264).

Light-inducible and/or diurnally-regulated promoters can be used to drive expression of a nucleotide sequence in a light-dependent manner. A light-responsive promoter can be, for example, a rbcS (ribulose-1,5-bisphosphate carboxylase) promoter which responds to light by inducing expression of an associated gene. In some cases, diurnally-regulated promoters can be used to drive expression of a nucleotide sequence in a manner regulated by light and/or the circadian clock. For example, a cab (chlorophyll a/b-binding) promoter can be used to produce diurnal oscillations in gene transcription. In some embodiments, a diurnally-regulated promoter can be a promoter region as disclosed in U.S. patent application Ser. No. 12/985,413, herein incorporated by reference. In some embodiments, a promoter can be used that drives expression of a nucleotide sequence in a diurnally-regulated manner but further with a temporal expression pattern opposite of that of endogenous ZmALDH7.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Parameters such as gene expression level, water use efficiency, drought tolerance, and others are typically presented with reference to a control cell or control plant. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null (“BN”). In another aspect, the present invention also provides methods for maintaining or increasing yield of a seed crop plant exposed to drought stress, where the methods include increasing expression of a polypeptide having at least 90% sequence identity to SEQ ID NO:2, or a variant or fragment thereof, in the plant. For example, methods may further comprise introducing into a target plant certain sequences which impact levels of lipid peroxidation under stress.

Nucleotide sequences encoding maize ZmALDH7 polypeptides and/or other polynucleotides of the present invention can be introduced into a plant. The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, breeding methods, stable transformation methods, transient transformation methods, and virus-mediated methods. “Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell targeted for transformation. For example, different methods may be preferred for use in monocots or in dicots. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722), and ballistic particle acceleration (see, for example, US Patent Numbers 4,945,050; U.S. Pat. No. 5,879,918; U.S. Pat. Nos. 5,886,244 and 5,932,782; Tomes, et al., (1995) in Plant Cell, Tissue, and Organ Culture Fundamental Methods, ed. Gamborg and Phillips, (Springer-Verlag, Berlin); McCabe, et al., (1988) Biotechnology 6:923-926); and Lec1 transformation (WO 2000/28058). See also, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and, 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

In specific embodiments, polynucleotide sequences of the invention can be provided to a plant using any of a variety of transient transformation methods. Such transient transformation methods include, but are not limited to, the introduction of the ZmALDH7 protein or variants and fragments thereof directly into the plant or the introduction of the ZmALDH7 transcript into the plant. Such methods include, for example, microinjection or particle bombardment. See, for example, Crossway, et al., (1986) Mol. Gen. Genet. 202:179-185; Nomura, et al., (1986) Plant Sci. 44:53-58; Hepler, et al., (1994) Proc. Natl. Acad. Sci. 91: 2176-2180 and Hush, et al., (1994) The Journal of Cell Science 107:775-784, all of which are herein incorporated by reference.

As indicated in some embodiments, the methods provided herein rely upon the use of Agrobacterium-mediated gene transfer to produce regenerable plant cells having a nucleotide sequence of interest. Agrobacterium-mediated gene transfer exploits the natural ability of Agrobacterium tumefaciens to transfer DNA into plant chromosomes. Agrobacterium is a plant pathogen that transfers a set of genes encoded in a region called T-DNA of the Ti plasmid into plant cells at wound sites. The typical result of gene transfer by the native pathogen is a tumorous growth called a crown gall in which the T-DNA is stably integrated into a host chromosome. The ability to cause crown gall disease can be removed by deletion of the genes in the T-DNA without loss of DNA transfer and integration. The DNA to be transferred is attached to border sequences that define the end points of an integrated T-DNA.

A variety of Agrobacterium species are known in the art, particularly for monocotyledon transformation. Such Agrobacterium can be used in the methods of the invention. See, for example, Hooykaas, (1989) Plant Mol. Biol. 13:327; Smith, et al., (1995) Crop Science 35:301; Chilton, (1993) Proc. Natl. Acad. Sci. USA 90:3119; Mollony, et al., (1993) N: Monograph Theor Appl Genet NY, Springer Verlag 19:148 and Ishida, et al., (1996) Nature Biotechnol. 14:745; Komari, et al. (1996) The Plant Journal 10:165, herein incorporated by reference. See, also, DNA Cloning Service on the world wide web at DNA-cloning.com.

The Agrobacterium strain utilized in the methods of the invention can be modified to contain a gene or genes of interest, or a nucleic acid to be expressed in the transformed cells. The nucleic acid to be transferred is incorporated into the T-region and is flanked by T-DNA border sequences. In the Ti plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid is transferred conjugatively in a tri-parental mating into A. tumefaciens which contains a compatible plasmid-carrying virulence gene. The vir functions are supplied in trans to transfer the T-DNA into the plant genome. Such binary vectors are useful in the practice of the present invention.

A vector comprising the nucleic acid of interest is introduced into an Agrobacterium. The term “introduced” is intended to mean providing a nucleic acid (e.g., expression construct) or protein into a cell (e.g., Agrobacterium). “Introduced” includes reference to the incorporation of a nucleic acid into a eukaryotic or prokaryotic cell where the nucleic acid may be incorporated into the genome of the cell and includes reference to the transient provision of a nucleic acid or protein to the cell. The term “introduced” includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA). General molecular techniques used in the invention are provided, for example, by Sambrook, et al., (eds.) Molecular Cloning: A Laboratory Manual, 1989, Cold Spring Harbor Laboratory Press, Cold Spring Harbor, N.Y.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the invention can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

In some cases, it is convenient to introduce nucleotide sequences of the invention as expression cassettes. Such expression cassettes can comprise 5′ and 3′ regulatory sequence operably linked to a ZmALDH7 polynucleotide of the invention or ABA-associated polynucleotide of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein-coding regions, contiguous and in the same reading frame. The expression cassette may additionally contain at least one additional gene to be cotransformed into the organism. Alternatively, additional gene(s) can be provided on multiple expression cassettes. Expression cassettes can be provided with a plurality of restriction sites for insertion of the gene of interest to be under the transcriptional regulation of the regulatory regions. The expression cassette may additionally contain selectable marker sequences.

In some embodiments, an expression cassette will include in the 5′-3′ direction of transcription, a transcriptional and translational initiation region (i.e., a promoter), a ZmALDH7 polynucleotide of the invention, and a transcriptional and translational termination region (i.e., termination region) functional in plants. The regulatory regions (i.e., promoters, transcriptional regulatory regions, and translational termination regions) and/or the ZmALDH7 polynucleotide of the invention may be native/analogous to the host cell or to each other. Alternatively, the regulatory regions and/or the ZmALDH7 polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

While it may be optimal to express the sequences using heterologous promoters, the native promoter sequences may be used. Such constructs can change expression levels of ZmALDH7 in the plant or plant cell. Thus, the phenotype of the plant or plant cell can be altered.

The termination region may be native with the transcriptional initiation region, may be native with the operably linked ZmALDH7 polynucleotide of interest, may be native with the plant host, or may be derived from another source (i.e., foreign or heterologous) to the promoter, the ZmALDH7 polynucleotide of interest, the plant host, or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference. The plant preferred codons may be determined from the codons of highest frequency in the proteins expressed in a monocot or dicot of interest. Likewise, the optimized sequence can be constructed using monocot-preferred or dicot-preferred codons. See, for example, Murray, et al., (1989) Nucleic Acids Res. 17:477-498. It is recognized that all or any part of the gene sequence may be optimized or synthetic. That is, fully optimized or partially optimized sequences may also be used.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20) and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments; other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In general, methods to modify or alter the host endogenous genomic DNA are available. This includes altering the host native DNA sequence or a pre-existing transgenic sequence including regulatory elements, coding and non-coding sequences. For example, the expression of the endogenous ALDH7 can be altered by site-specific modification of the endogenous promoter driving the expression of ALDH7. By way of illustration, an enhancer element can be engineered into the endogenous promoter such that the expression is increased. In another aspect, one or more site-directed mutations may result in increased expression. These methods are also useful in targeting nucleic acids to pre-engineered target recognition sequences in the genome. As an example, the genetically modified cell or plant described herein, is generated using “custom” meganucleases produced to modify plant genomes (see, e.g., WO 2009/114321; Gao, et al., (2010) Plant Journal 1:176-187). Another site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41. A transcription activator-like (TAL) effector-DNA modifying enzyme (TALE or TALEN) is also used to engineer changes in plant genome. See, e.g., US Patent Application Publication Number 2011/0145940, Cermak, et al., (2011) Nucleic Acids Res. 39(12) and Boch, et al., (2009) Science 326(5959):1509-12.

The ZmALDH7 polypeptides described herein may be used alone or in combination with additional polypeptides or agents to increase drought stress tolerance in plants. For example, in the practice of certain embodiments, a plant can be genetically manipulated to produce more than one polypeptide associated with increased drought tolerance. Those of ordinary skill in the art realize that this can be accomplished in any of a number of ways. For example, each of the respective coding sequences for polypeptides described herein can be operably linked to a promoter and then joined together in a single continuous DNA fragment comprising a multigenic expression cassette. Such a multigenic expression cassette can be used to transform a plant to produce the desired outcome. Alternatively, separate plants can be transformed with expression cassettes containing one or a subset of the desired coding sequences. Transformed plants that exhibit the desired genotype and/or phenotype can be selected by standard methods available in the art such as, for example, immunoblotting using antibodies which bind to the proteins of interest, assaying for the products of a reporter gene, and the like. Then, all of the desired coding sequences can be brought together into a single plant through one or more rounds of cross-pollination utilizing the previously selected transformed plants as parents.

Methods for cross-pollinating plants are well known to those skilled in the art and are generally accomplished by allowing the pollen of one plant, the pollen donor, to pollinate a flower of a second plant, the pollen recipient and then allowing the fertilized embryos in the pollinated flower to mature into seeds. Progeny containing the entire complement of desired coding sequences of the two parental plants can be selected from all of the progeny by standard methods available in the art as described supra for selecting transformed plants. If necessary, the selected progeny can be used as either the pollen donor or pollen recipient in a subsequent cross-pollination. Selfing of appropriate progeny can produce plants that are homozygous for both added, heterologous genes. Back-crossing to a parental plant and out-crossing with a non-transgenic plant are also contemplated, as is vegetative propagation. Descriptions of other breeding methods that are commonly used for different traits and crop plants can be found in several references, e.g., Fehr, (1987), Breeding Methods for Cultivar Development, ed. J. Wilcox (American Society of Agronomy, Madison, Wis.).

The present invention may be used for transformation of any plant species, including, but not limited to, monocots and dicots. In some cases, plant species useful in the methods provided herein can be seed crop plants such as grain plants, oil-seed plants, and leguminous plants. Of particular interest are plants where the seed is produced in high amounts, or the seed or a seed part is edible. Seeds of interest include the grain seeds such as wheat, barley, rice, corn (maize), rye, millet, and sorghum. Plants of particular interest are corn, wheat, and rice.

Examples of plant species of interest include, but are not limited to, corn (maize; Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus effiotii), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The article “a” and “an” are used herein to refer to one or more than one (i.e., to at least one) of the grammatical object of the article. By way of example, “an element” means one or more element.

The following examples are presented by way of illustration, and not by way of limitation.

### EXPERIMENTAL

### Example 1

### Identification and Characterization of ZmALDH7

### Sequence Analysis

Soy ALDH7 has been identified (Rodrigues, et al., (2006) J. Exp. Bot. 57:1909-1918. A maize orthologue of the soybean gene GmTP55 called ZM-ALDH7 (p0113.cieac66ra:fis) was identified using proprietary search and bioinformatics applications.

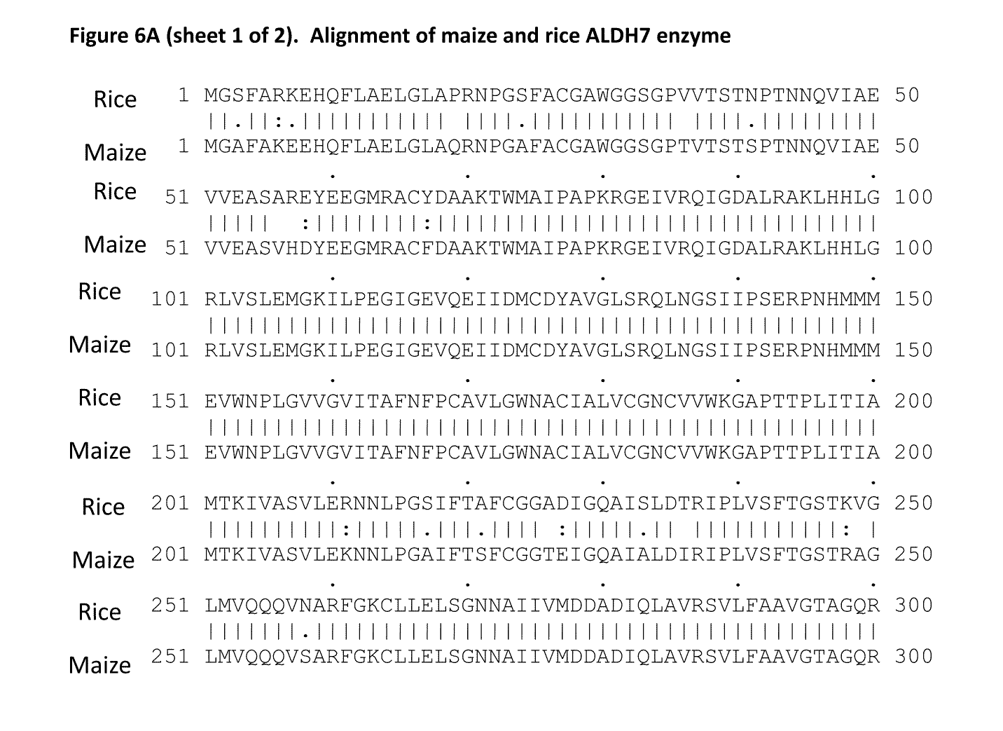

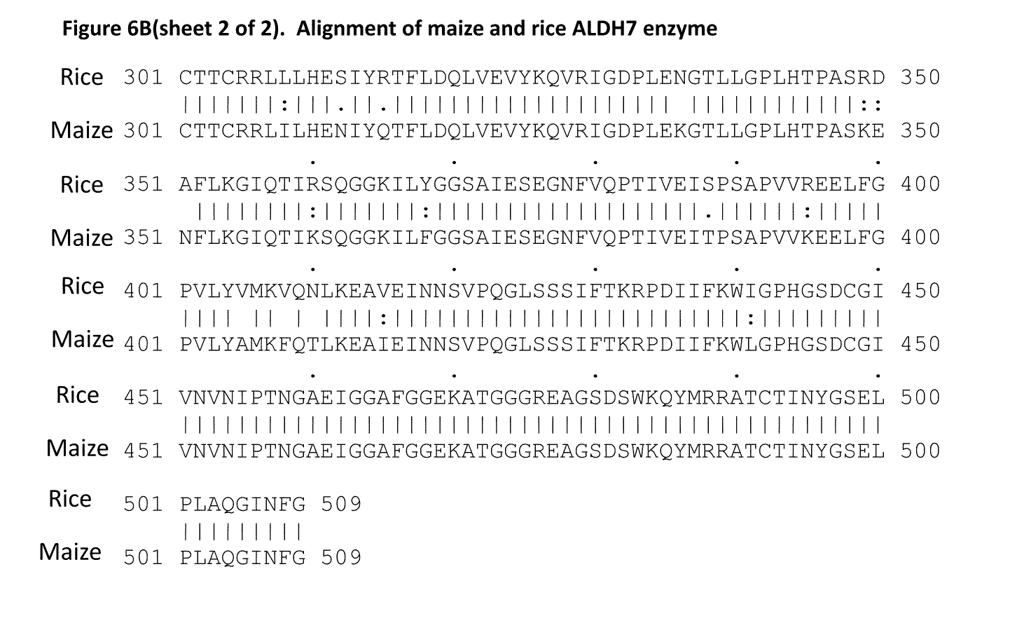

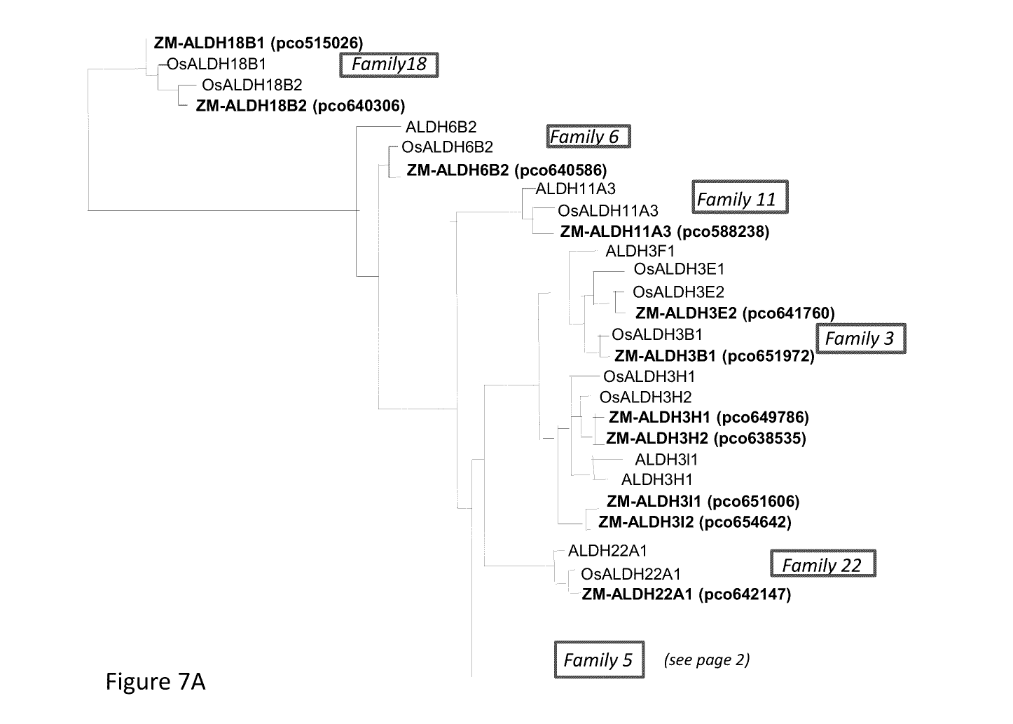

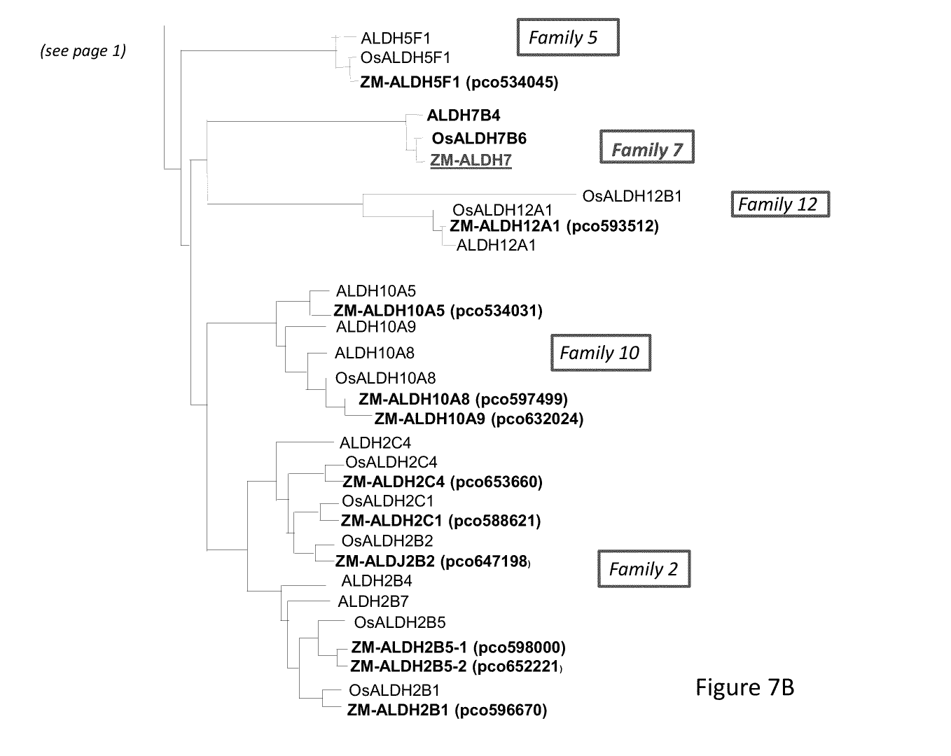

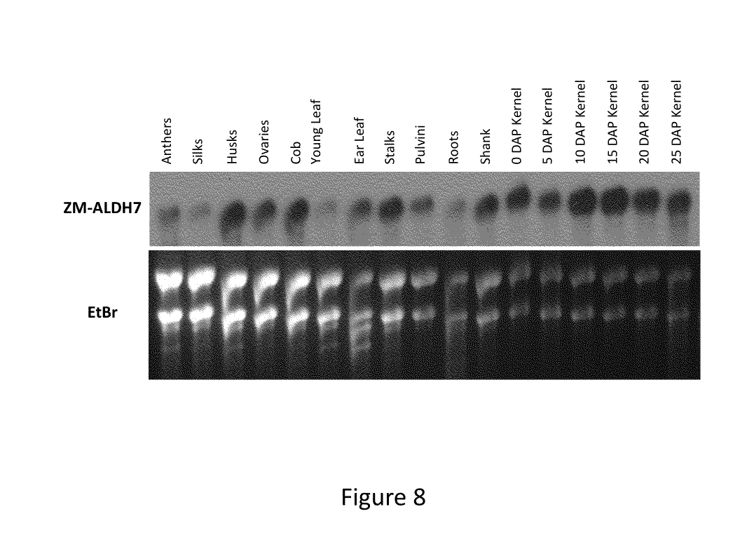

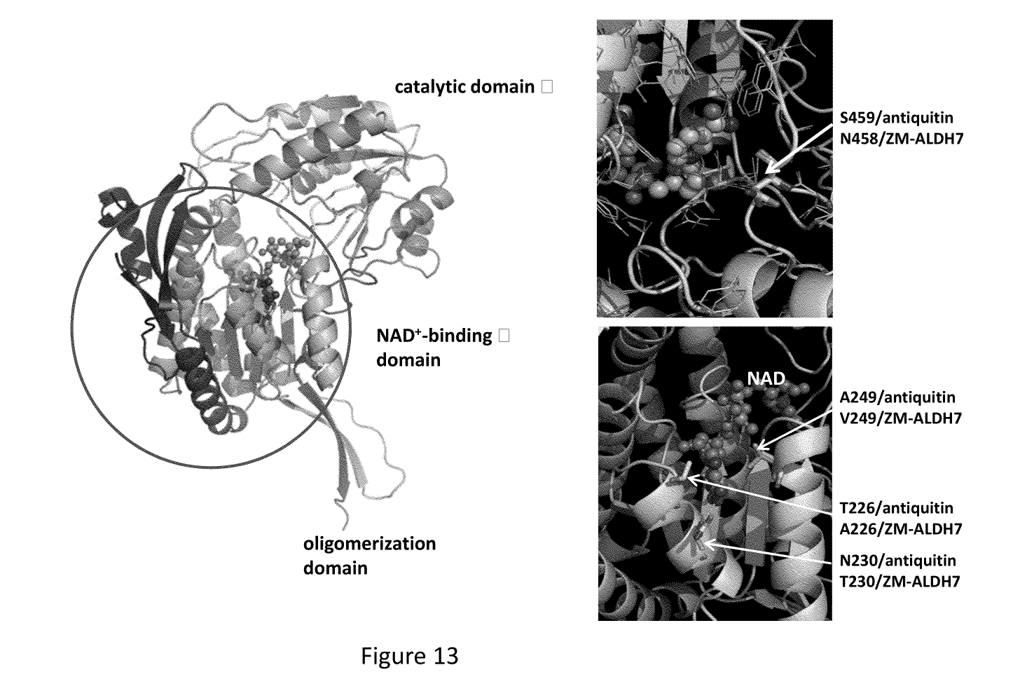

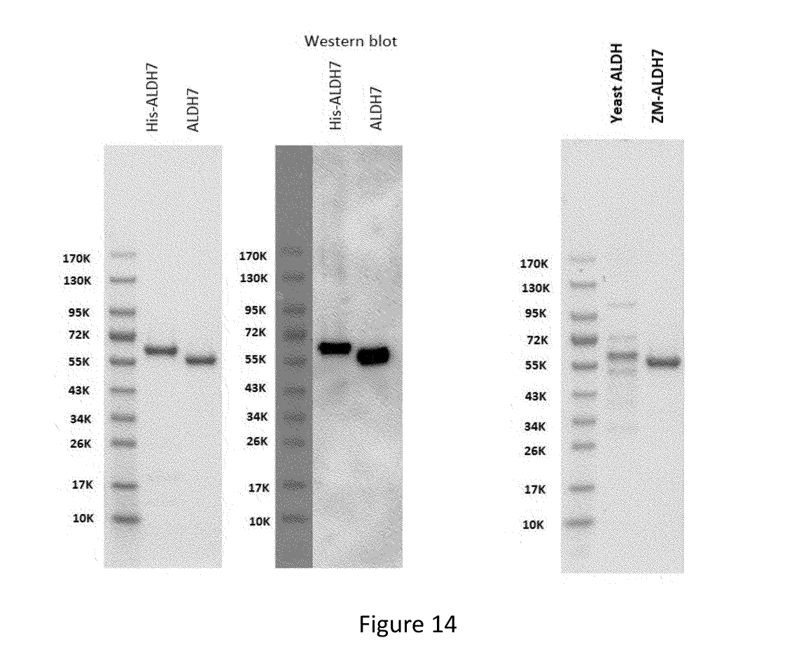

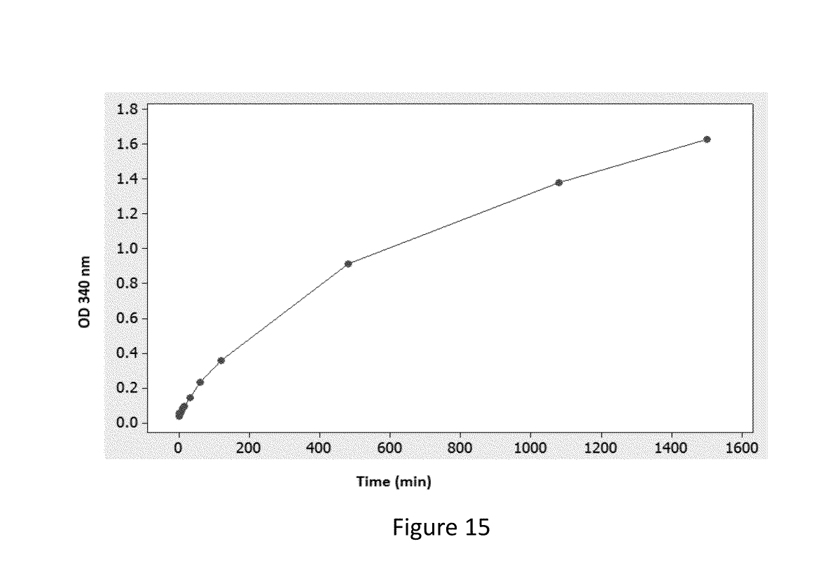

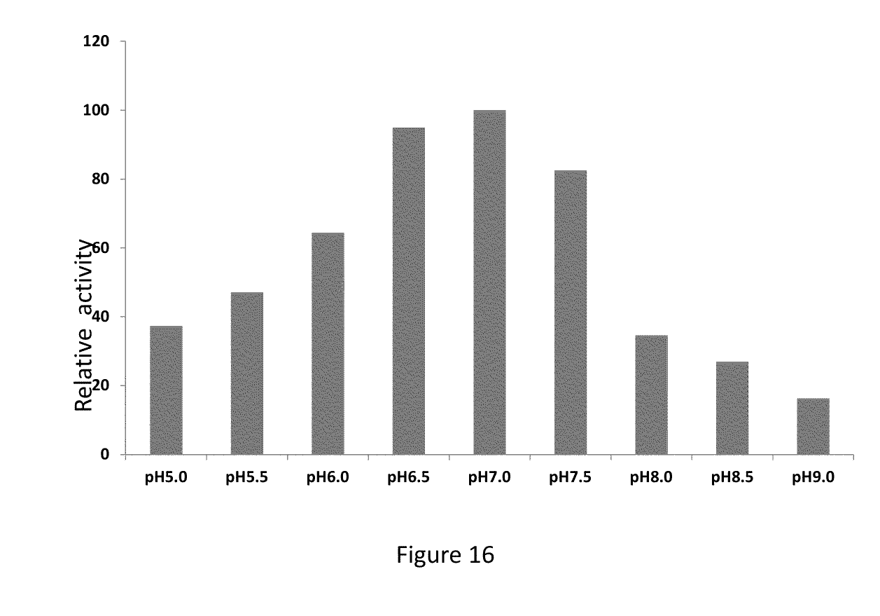

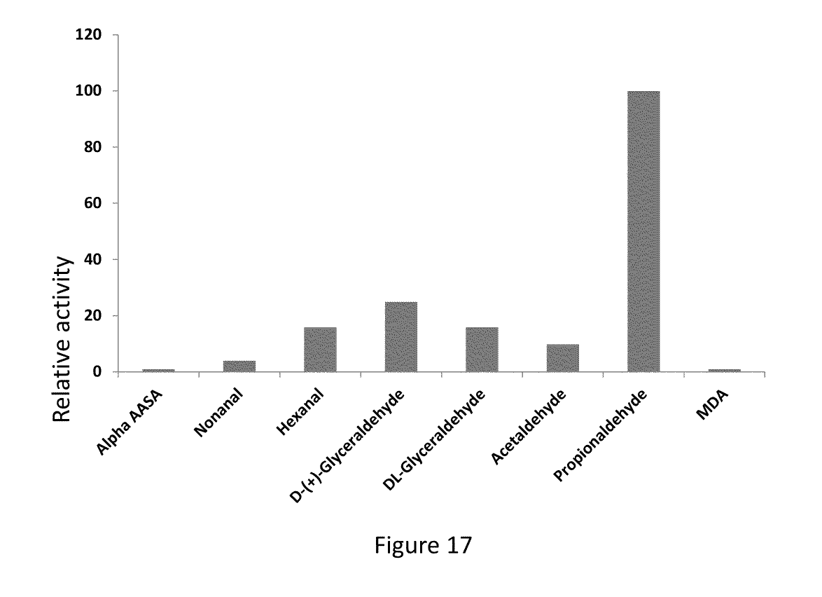

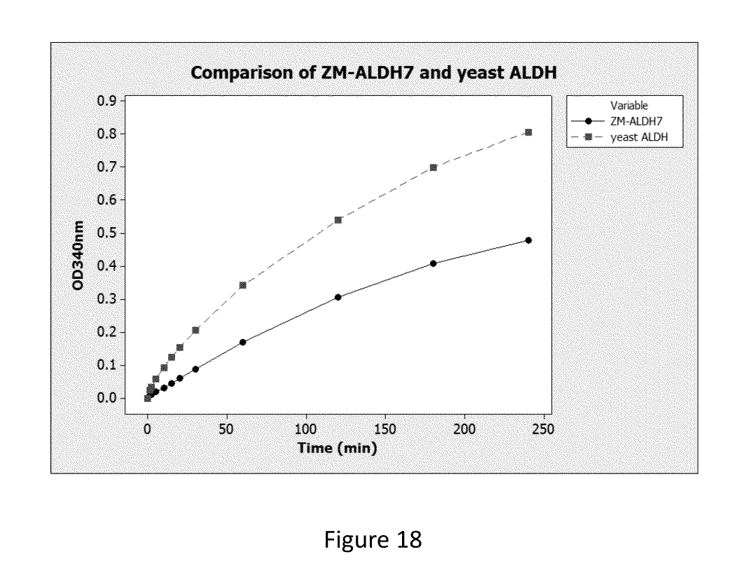

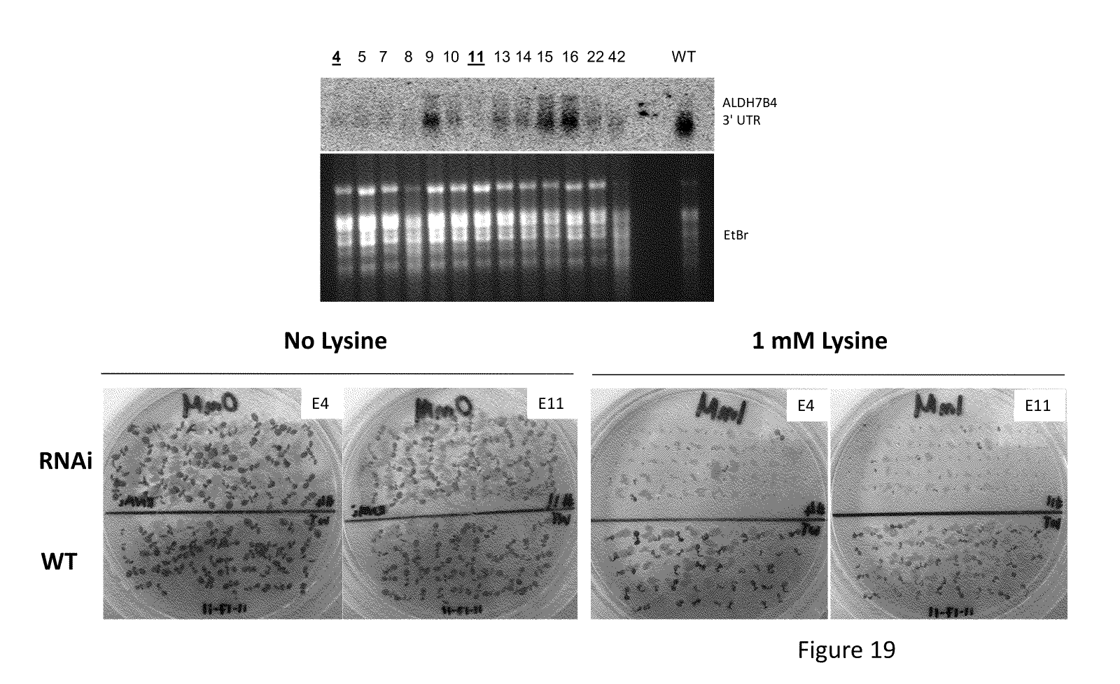

An alignment of maize and rice ALDH7 polypeptides is provided at FIG. 6. ZmALDH7 is 92.7% identical and 97.6% similar to OsALDH7. FIG. 13 provides protein modeling results which indicate conservation of active sites between ZmALDH7 and antiquitin from Seabream.

### Characterization of ZmALDH7 Gene Expression

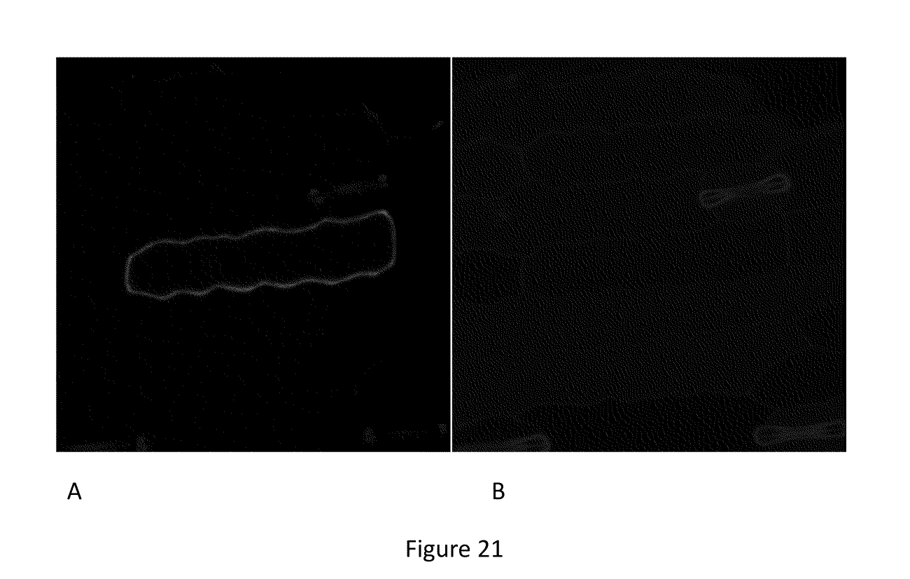

Data indicate that ZmALDH7 may be located in the cytoplasm and be membrane-bound. See, FIG. 21 showing apparent cytoplasmic localization of ZmALDH7:AcGFP1 translational fusion in maize leaf epidermis pavement cell. Bioinformatic prediction using ProtComp v6.1 indicates the protein could be membrane bound. This cellular location would be consistent with a role in detoxification of lipid-peroxidation-derived reactive aldehydes, providing oxidative stress tolerance.

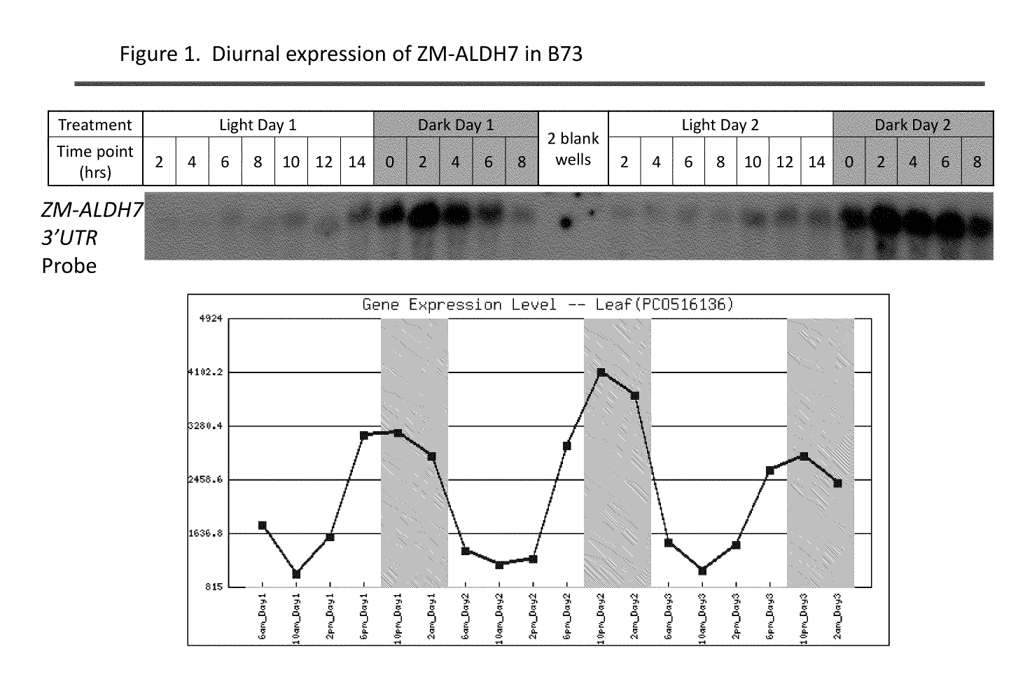

Native expression of ZmALDH7 in the leaf shows a strong diurnal pattern, with a peak in the evening (˜10 p.m.) and lowest level in late morning (˜10 a.m.). See, FIG. 1. It may be useful to express ZmALDH7 under control of a diurnally-regulated promoter which increases expression during the day, when drought stress may be most severe.

### Drought or ABA Induction of ZmALDH7 Gene Expression

Expression of ZmALDH7 in root, leaf, and immature ear tissue was assayed, as follows.

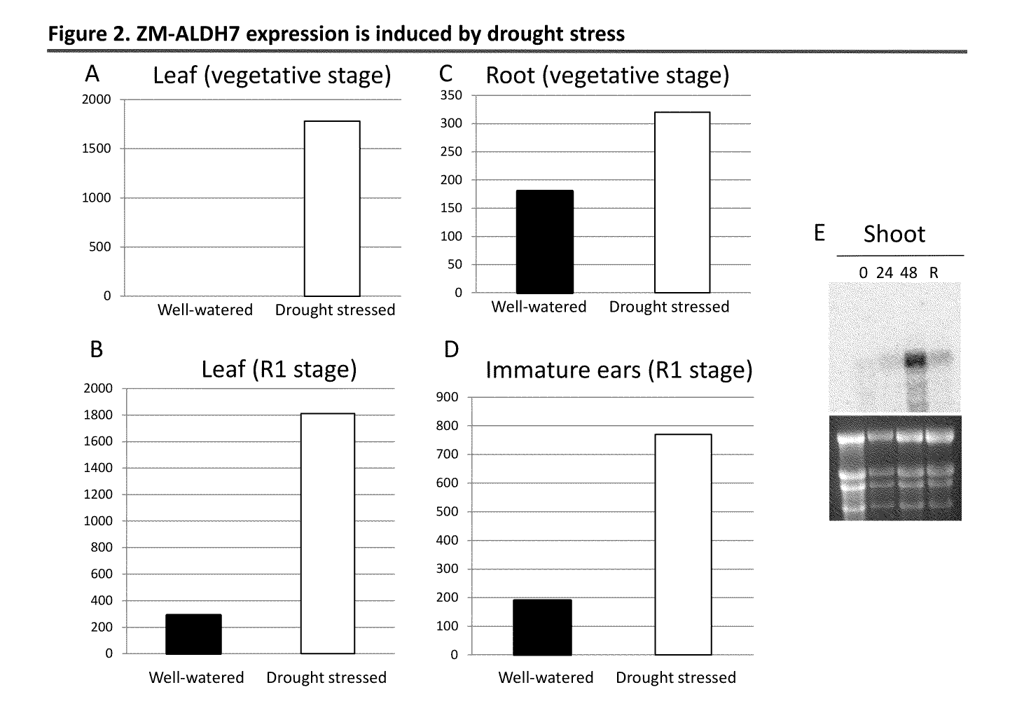

Maize plants, grown under standard greenhouse conditions, were either watered on a schedule (control) or water was withheld (drought stressed). Watering of controls was twice a day for 5 minutes on an automatic watering system, and was continued throughout the whole experiment. Plants were watered for the first month, then transplanted (along with controls) into large buckets, then water was withheld. Two weeks after withholding water, tissue was collected. For both controls and drought stressed plants, leaf tissue and root tissue were separately collected. Tissue from two plants was pooled for each sample. Expression levels were measured using MPSS (FIG. 2, vegetative stage). A strong induction of ZmALDH7 expression was identified in leaf and a two-fold increase in root indicating that ZmALDH7 is responsive to drought stress in both root and shoot of maize plants during vegetative development.

Expression at flowering stage R1 was measured using 3234 hybrid plants grown in the field. The drought stress period was imposed starting 5 weeks pre-anthesis (650-700 GDU). Ear leaves and immature ears were from well-watered and drought stressed plots. Expression was measured using MPSS (FIG. 2, R1 stage). A strong induction of ZmALDH7 expression was observed in leaf and immature ears of field grown plants under drought stress. The results indicate that induction of ZmALDH7 gene expression by drought stress can also be observed at flowering.

Seedlings were grown to stage V6 in Turface®. Samples were harvested after last watering (time point 0) and plants were drought-stressed by withholding water for 24 hours (24) and 48 hours (48). After 48 hours, plants were re-watered; samples were harvested from shoot, root, leaf, and immature ear tissue 24 hours after recovery (R). Total RNA extracted from plant samples were used in a Northern blot experiment with a ZmALDH7 radioactive sprobe. A strong induction of ZmALDH7 expression was found after 48 h f stress. ZmALDH7 transcript levels return close to normal level 24 h after rewatering (FIG. 2E). This experiment indicates that the effect of drought stress on ZmALDH7 expression levels can be reversed when stress is alleviated,

Induction of ZmALDH7 by ABA was studied as follows.

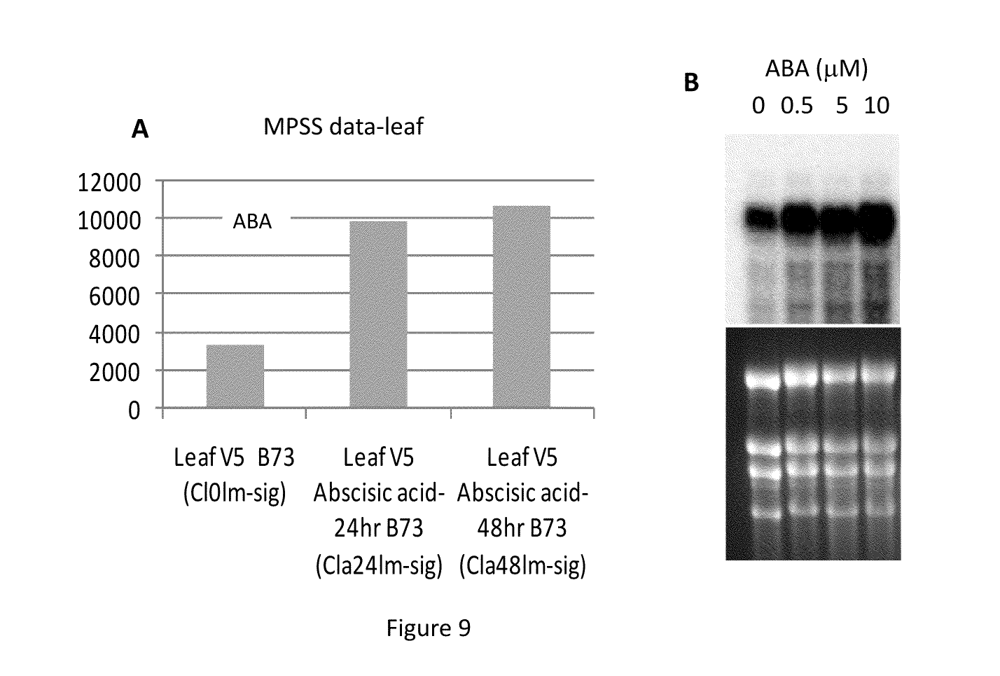

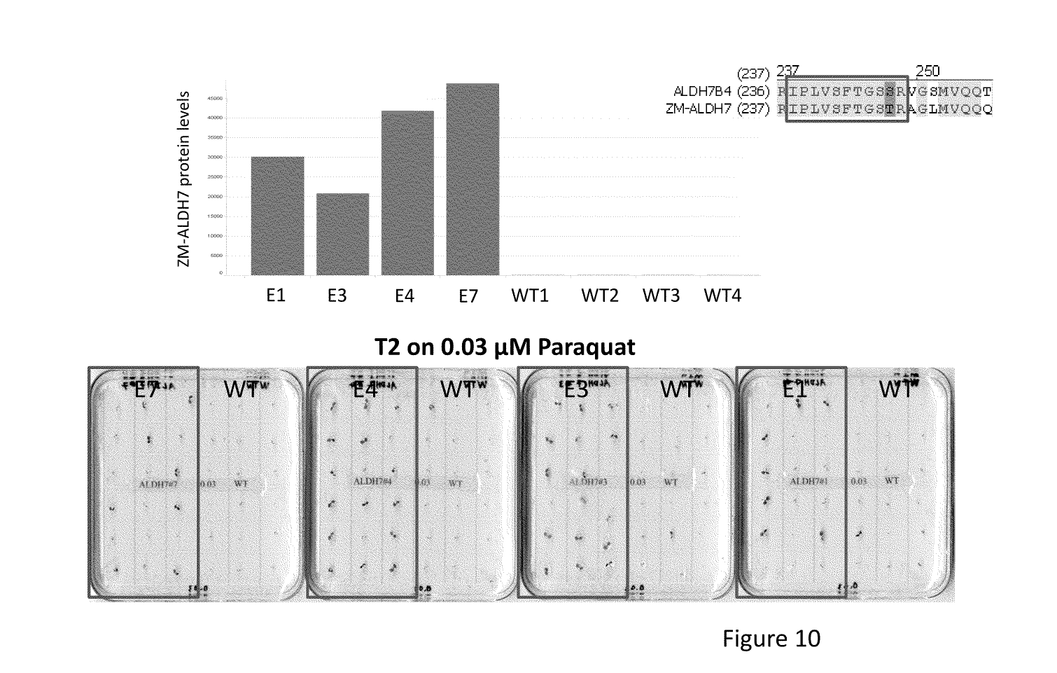

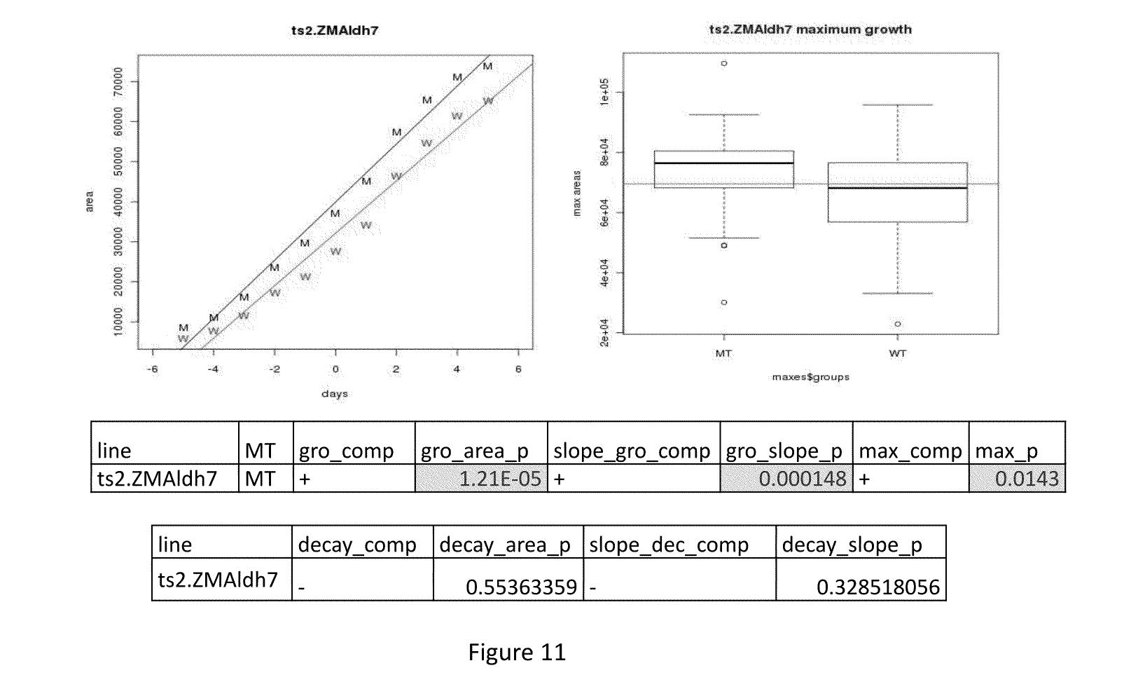

Greenhouse grown V5 stage B73 maize plants were treated with 0.1 mM ABA and leaves from six plants were harvested after 0, 24 and 48 hrs of treatment. Expression of ZmALDH7 was measured using MPSS. A 3-fold induction was observed after 24 h and induction persisted after 48 h of treatment (FIG. 9A).

Leaf discs (˜7 mm in diameter) were obtained from maize B73 leaf using a leaf puncher. Discs were floated on solution containing 0, 0.5, 5, or 10 μM ABA for 24 hours under constant light. Total RNA were extracted and used for Northern with a ZmALDH7 radioactive probe. Results confirmed that ZmALDH7 expression is induced by ABA. The data further indicate that the induction of ZmALDH7 expression in maize leaf by ABA is dose dependent as higher concentration of ABA results in stronger expression levels (FIG. 9B).

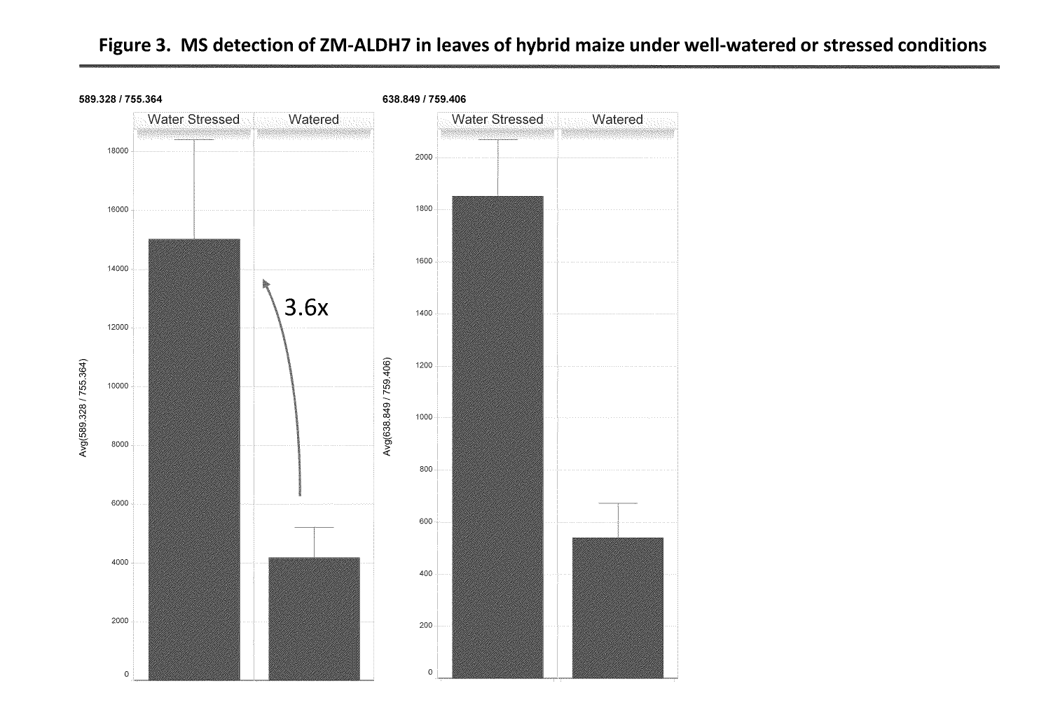

Stress-induced expression of ZmALDH7 in hybrid maize was further confirmed studied at the protein level using mass spectrometry measurement of two ZmALDH7 peptides as shown in FIG. 3. Plants at V6-V7 stage were subjected to 30 hours of drought stress. Data indicate increased levels of the native protein under drought conditions (“water stressed”) compared to well-watered conditions (“watered”). ZmALDH7 increase in leaves was greater than 3-fold.

### Example 2

### Over-Expression of ZmALDH7 in Maize

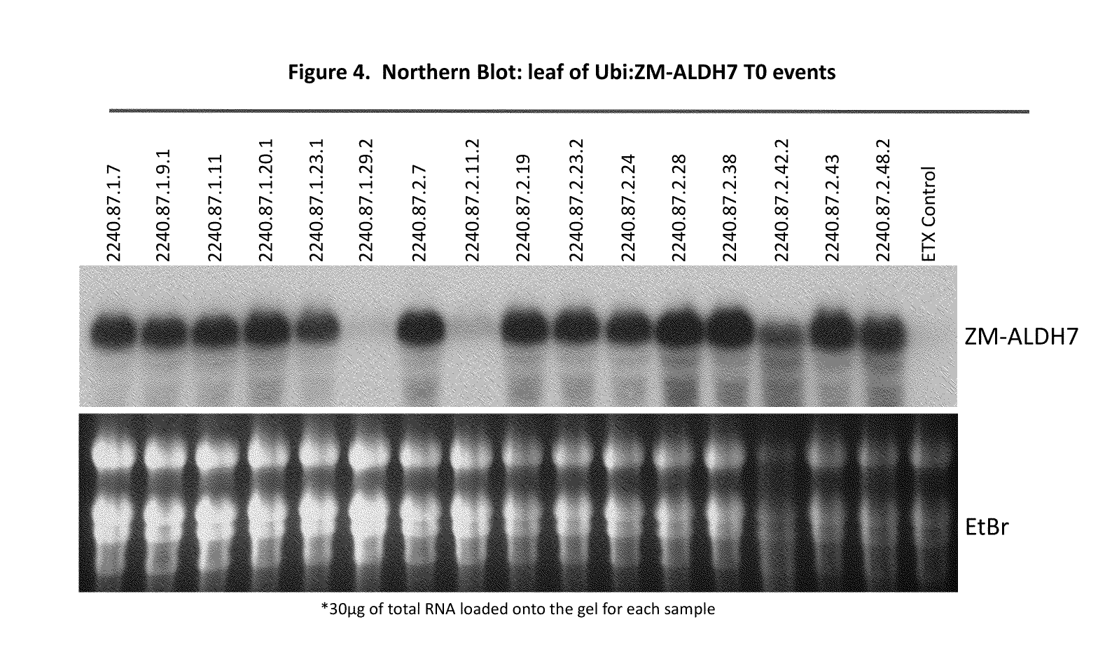

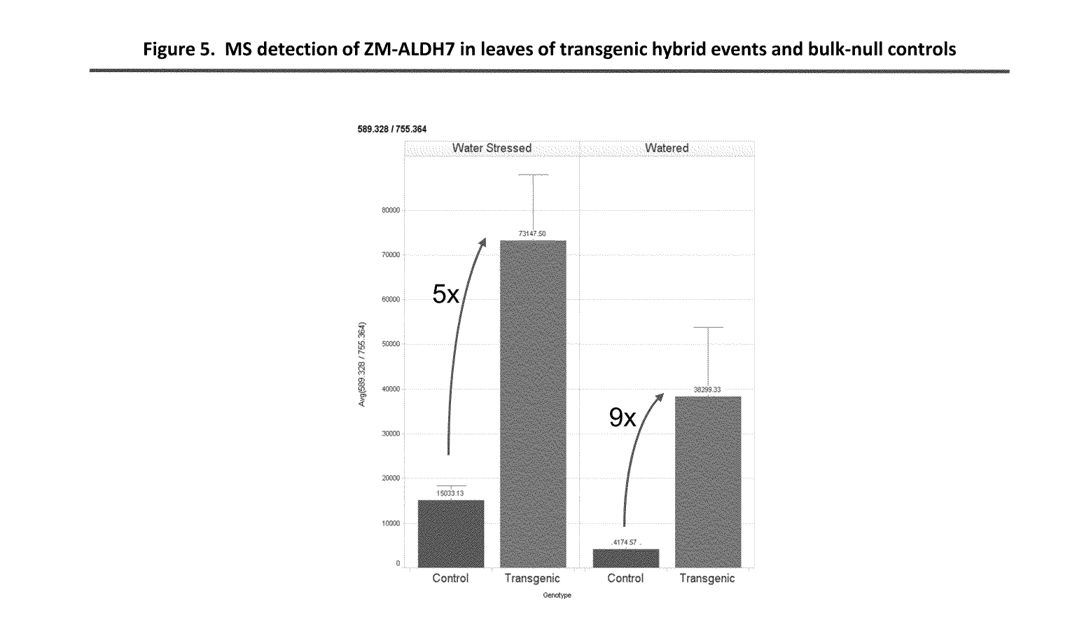

Maize embryos were transformed as described in Example 6 with a construct comprising the ubiquitin promoter driving ZmALDH7 (SEQ ID NO: 1). FIG. 4 shows elevated ZmALDH7 expression in 14 of 16 transgenic events, as determined by Northern blot.

Measurement of the ZmALDH7 protein in transgenic events via mass spectrometry showed a 5-fold increase with respect to expression in bulk-null control plants under water stress and a 9 fold increase in well-watered condition.

Under flowering stress, maize plants transgenic for Ubi:ZmALDH7 had a higher number of green leaves per plant than either control plants (bulk null) or plants comprising an alternative construct. On average, the Ubi:ZmALDH7 transgenic plants had an average of 2.63 green leaves compared to an average of 2 for controls. This equates to 2 out of 3 transgenic plants having one more green leaf than the bulk null in average, under flowering-stress conditions.

### Example 3

### Increased Grain Yield by Maize Overexpressinq ZmALDH7

Maize events overexpressing ZM-ALDH7 under the control of a constitutive maize UBI1 promoter were evaluated for improvement in drought tolerance.

Year one: Six out of 8 individual events tested in hybrid combination under mild drought stress showed a significant (p<0.1) yield increase (average of 6.1 bu/acre) over bulk null controls. One event showed significant yield improvement under grain fill stress. No significant differences with respect to controls were detected in two locations under flowering stress conditions.

Year two: Events were re-evaluated in the same hybrid combination. Five out of 8 events showed a significant increase in yield performance under flowering stress (average 4.3 bu/acre improvement (p<0.1) in one test location. No significant differences were identified under grain fill stress or in two other test locations.

Year three: Events were evaluated using in three hybrid combinations. A positive effect on yield was observed across testers and locations, with more noticeable effects in the high-yield and very-low-yield locations.

### Example 4

### Classification of ALDH7 into Subfamilies

Using proprietary materials, global homologs for ALDH7 were assembled and aligned to generate phylogenetic tree of the ALDH7 gene family. Briefly, the phylogenetic tree was generated using sequences that share <60% sequence identity. The sequences from each of the clusters in the tree containing ≧3 members with a bootstrap confidence of >60 were used to create HMM profiles specific to each cluster. Further, these HMM profiles were used for classifying homologs in ALDH gene family to corresponding subfamilies.

ALDH could be divided into 200 subgroups based on this classification. ZmALDH7 clustered in group 19 along with ALDH7B4 (A. thaliana) and LOC_Os09g26880 (Rice). Group 19 contains close to 30 members.

ALDH7 Subfamily Members from Stress Tolerant Plants and Moss.

In addition to plant and mammalian ALDH7 homologs which clustered under group 19, homologs from several species that tolerate adverse conditions have been identified. A brief description of the species and the corresponding ALDH7 members is listed below.
1. ALDH7 homolog from Thelungiella halophila (SEQ ID NO: 64-65) (E4MXX4_THEHA). Thellungiella species have been studied for their ability to function in extreme salt, cold and freezing conditions and for efficient mobilization of resources in poor or degraded soils. A comparative study of 11 Brassicas suggests that T. parvula may perform slightly better than T. salsuginea under salt and drought conditions, but the two are comparable in cold and freezing responses.
2. ALDH7 homolog from Euphorbia characias (SEQ ID NO: 61-62) (Q5EBY6_EUPCH). It grows in the form of a shrub or bush with many stems and characteristic black or dark brown nectar glands in the cyathia. The fruits are smooth capsules. It is a tough perennial plant, capable of resisting long periods of drought. It grows preferably in dry areas, often far away from the freatic sheet, both in flat as well as in mountainous terrain. This plant can also resist high salinity.
3. ALDH7 homolog from Picea sitchensis (SEQ ID NO: 65-66) (B8LS13_PICSI) Sitka spruce is of major importance in forestry for timber and paper production outside of its native range, it is particularly valued for its fast growth on poor soils and exposed sites where few other trees can be grown successfully. It is more tolerant to wind and saline ocean air, and grows faster,than the native Norway spruce.
4. ALDH7 homolog from Tortula ruralis (SEQ ID NO: 67-68) (Q8RYB7_TORRU) Tortula ruralis is a species of moss. Common names include twisted moss and star moss. It grows in many types of climate, including the Arctic, boreal areas, temperate areas and deserts. It tolerates a variety of elevations and levels of sunlight. It helps to stabilize soil and reduce erosion. It can dry out and become dormant for many years, becoming metabolically active again after many decades of desiccation.
5. ALDH7 homolog from Setaria italica (SEQ ID NO: 69-70)

The group 19 subfamily has no bacterial homologs. However, homologs from abiotic stress resistant species described above are potential drought-tolerance candidates.

### Example 5

### Biolistic Transformation and Regeneration of Transgenic Maize

Immature maize embryos from greenhouse donor plants are bombarded with a plasmid containing the ZmALDH7 gene operably linked to a promoter and the selectable marker gene PAT (Wohlleben, et al., (1988) Gene 70:25-37), which confers resistance to the herbicide bialaphos. Alternatively, the selectable marker gene is provided on a separate plasmid. Transformation is performed as follows. Media recipes follow below.

### Preparation of Target Tissue

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising a ZmALDH7 gene operably linked to a promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for ABA levels and/or drought tolerance.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose and 1.0 ml/l of 0.1 mM abscisic acid (brought to volume with polished D-I H₂O after adjusting to pH 5.6); 3.0 g/l Gelrite (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/l myo-inositol, and 40.0 g/l sucrose (brought to volume with polished D-I H₂O after adjusting pH to 5.6) and 6 g/l bacto-agar (added after bringing to volume with polished D-I H₂O), sterilized and cooled to 60° C.

### Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

Regenerated plants are referred to as the T0 (T-zero) generation. Subsequent generations are T1, T2, and so forth.

### Example 6

### Agrobacterium-Mediated Transformation

For Agrobacterium-mediated transformation of maize with a ZmALDH7 polynucleotide sequence of the invention, the method of Zhao is employed (U.S. Pat. No. 5,981,840, and PCT Patent Publication Number WO 1998/32326, the contents of which are hereby incorporated by reference; see, also, Zhao, et al., (1998) Maize Genetics Cooperation Newsletter 72:34-37). Briefly, immature embryos are isolated from maize and the embryos contacted with a suspension of Agrobacterium, where the bacteria are capable of transferring the ZmALDH7 polynucleotide of interest to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step) and calli grown on selective medium are cultured on solid medium to regenerate the plants.

### Example 7

### Soybean Embryo Transformation

### Culture Conditions

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein, et al., (1987) Nature 327:70).

### Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed is cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

### Preparation of DNA for Bombardment

Either an intact plasmid or a DNA plasmid fragment containing the genes of interest and the selectable marker gene are used for bombardment. Plasmid DNA for bombardment are routinely prepared and purified using the method described in the Promega™ Protocols and Applications Guide, Second Edition (page 106). Fragments of the plasmids carrying the ZmALDH7 polynucleotide of interest are obtained by gel isolation of double digested plasmids. In each case, 100 ug of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing ZmALDH7 polynucleotide of interest are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e. per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

### Selection of Transformed Embryos

Transformed embryos were selected either using hygromycin (when the hygromycin phosphotransferase, HPT, gene was used as the selectable marker) or chlorsulfuron (when the acetolactate synthase, ALS, gene was used as the selectable marker).

### Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

### Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos into Plants

In order to obtain whole plants from embryogenic suspension cultures, the tissue must be regenerated.

### Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 μE/m²s. After this time embryo clusters are removed to a solid agar media, SB166, for 1-2 weeks. Clusters are then subcultured to medium SB103 for 3 weeks. During this period, individual embryos can be removed from the clusters and screened for ABA accumulation. It should be noted that any detectable phenotype, resulting from the expression of the genes of interest, could be screened at this stage.

### Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins.

### Media Recipes

SB 196 - FN Lite liquid proliferation medium (per liter) -

| | | |
|---|---|---|
| MS FeEDTA - 100x Stock 1 | 10 | ml |
| MS Sulfate - 100x Stock 2 | 10 | ml |
| FN Lite Halides - 100x Stock 3 | 10 | ml |
| FN Lite P, B, Mo - 100x Stock 4 | 10 | ml |
| B5 vitamins (1 ml/L) | 1.0 | ml |
| 2,4-D (10 mg/L final concentration) | 1.0 | ml |
| KNO₃ | 2.83 | gm |
| (NH₄)₂ SO₄ | 0.463 | gm |
| Asparagine | 1.0 | gm |
| Sucrose (1%) | 10 | gm |

pH 5.8

FN Lite Stock Solutions

| Stock # | | 1000 ml | | 500 ml | |
|---|---|---|---|---|---|
| 1 | MS Fe EDTA 100x Stock | | | | |
| | Na₂ EDTA* | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO—2H₂O | 0.025 | g | 0.0125 | g |

*Add first, dissolve in dark bottle while stirring

SB1 solid medium (per liter) comprises: 1 pkg. MS salts (Gibco/BRL—Cat#11117-066); 1 ml B5 vitamins 1000× stock; 31.5 g sucrose; 2 ml 2,4-D (20 mg/L final concentration); pH 5.7 and 8 g TC agar.

SB 166 solid medium (per liter) comprises: 1 pkg. MS salts (Gibco/BRL—Cat#11117-066); 1 ml B5 vitamins 1000× stock; 60 g maltose; 750 mg MgC12 hexahydrate; 5 g activated charcoal; pH 5.7 and 2 g gelrite.

SB 103 solid medium (per liter) comprises: 1 pkg. MS salts (Gibco/BRL—Cat#11117-066); 1 ml B5 vitamins 1000× stock; 60 g maltose; 750 mg MgC12 hexahydrate; pH 5.7 and 2 g gelrite.

SB 71-4 solid medium (per liter) comprises: 1 bottle Gamborg's B5 salts w/ sucrose (Gibco/BRL—Cat#21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20 C comprises: 10 g myo-inositol; 100 mg nicotinic acid; 100 mg pyridoxine HCl; and, 1 g thiamine. If the solution does not dissolve quickly enough, apply a low level of heat via the hot stir plate. Chlorsulfuron Stock comprises 1 mg/ml in 0.01 N Ammonium Hydroxide.

### Example 8

### Sunflower Meristem Tissue Transformation

Sunflower meristem tissues are transformed with an expression cassette containing the ZmALDH7 polynucleotide operably linked to a promoter as follows (see also, European Patent Number EP 0 486233, herein incorporated by reference and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer, et al. (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al. (1962) Physiol. Plant. 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6 and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the ZmALDH7 gene operably linked to the promoter is introduced into Agrobacterium strain EHA105 via freeze-thawing as described by Holsters, et al., (1978) Mol. Gen. Genet. 163:181-187. The plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for ZmALDH7 activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by ZmALDH7 activity analysis of leaf extracts while transgenic seeds harvested from NPTII-positive T₀ plants are identified by ZmALDH7 activity analysis of small portions of dry seed cotyledon.

### Example 9

### High Throughput Drought-Tolerance Screens

Plant performance under drought may be evaluated in a high-throughput fashion using, for example, LemnaTec imaging (LemnaTec GmbH, Wurselen, Germany). Water use efficiency measurements are calculated by normalizing plant biomass or yield with respect to the amount of water used during the growing period of interest.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

We claim:

1. A method for increasing drought tolerance in a plant, said method comprising:
a) introducing into said plant a polynucleotide construct comprising a nucleotide sequence encoding a polypeptide selected from the group consisting of:
a. a polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
b. a polypeptide having at least 95% sequence identity to a polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
c. a polypeptide having at least 90% sequence identity to a polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
wherein said nucleotide sequence is operably linked to a heterologous promoter; and
b) expressing said nucleotide sequence in said plant,
whereby drought tolerance of said plant is increased relative to a control plant.

2. The method of claim 1, wherein said promoter is selected from the group consisting of a weak constitutive promoter, an organ-preferred or tissue-preferred promoter, a stress-inducible promoter, a chemical-inducible promoter, a light-responsive promoter, and a diurnally-regulated promoter.

3. The method of claim 2, wherein said tissue-preferred promoter is a leaf-preferred promoter, a root-preferred promoter, a vasculature-specific promoter or a promoter without expression in developing or mature ears.

4. The method of claim 2, wherein said stress-inducible promoter is a Rab17 promoter or an Rd29a promoter.

5. The method of claim 2, wherein said light-responsive promoter is an rbcS (ribulose-1,5-bisphosphate carboxylase) promoter, a Cab (chlorophyll a/b-binding) promoter or a phosphoenol-pyruvate carboxylase (PEPc) promoter.

6. The method of claim 1, wherein said nucleotide sequence encodes a polypeptide having at least 95% sequence identity to the full length of SEQ ID NO: 2.

7. A method for increasing yield of a seed crop plant exposed to drought stress, said method comprising increasing expression in said plant of a polypeptide selected from the group consisting of:
a. SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
b. a polypeptide at least 95% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74 and
c. a polypeptide at least 90% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72, and 74.

8. The method of claim 7, wherein lipid peroxidation under drought stress is reduced.

9. The method of claim 7, wherein accumulation of malondialdehyde under drought stress is reduced.

10. The method of claim 7, wherein increased expression occurs primarily during night.

11. The method of claim 7, wherein said seed crop plant is selected from the group consisting of a grain plant, an oil-seed plant and a leguminous plant.

12. The method of claim 11, wherein said grain plant is maize or wheat.

13. A plant comprising a polynucleotide construct comprising a nucleotide sequence encoding a polypeptide selected from the group consisting of:
a. SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
b. a polypeptide at least 95% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74 and
c. a polypeptide at least 90% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72, and 74,
wherein said nucleotide sequence is operably linked to a heterologous promoter selected from the group consisting of a weak constitutive promoter, an organ-preferred or tissue-preferred promoter, a stress-inducible promoter, a chemical-inducible promoter, a light-responsive promoter and a diurnally-regulated promoter.

14. The plant of claim 13, wherein said plant is a seed crop plant.

15. The plant of claim 13, wherein said plant exhibits an increase in drought tolerance relative to a control plant.

16. A transformed seed of the plant of claim 13.

17. The method of claim 7, wherein said plant is maize and wherein the grain yield of a plurality of said plants exceeds 150 bushels per acre.

18. The plant of claim 13, wherein said plant is maize and wherein the grain yield of a plurality of said plants is at least 3% greater than the yield of a plurality of control plants.

19. The plant of claim 18, wherein said yield increase occurs under drought conditions.

20. A method for increasing drought tolerance in a plant, said method comprising introducing into said plant a polynucleotide construct comprising a nucleotide sequence encoding a polypeptide which directs, expands, amplifies, or accelerates the degradation of aldehydes or results in the reduced accumulation of aldehydes in the plant, and expressing said polynucleotide in said plant, wherein drought tolerance of said plant is increased relative to a control plant.

21. The method of claim 20, wherein said polynucleotide is selected from the group consisting of:
a. SEQ ID NO: 1, 61, 63, 65, 67, 69, 71, 73, 75 and 76;
b. a polynucleotide at least 95% identical to any of SEQ ID NO: 1, 61, 63, 65, 67, 69, 71, 73, 75 and 76;
c. a polynucleotide at least 90% identical to any of SEQ ID NO: 1, 61, 63, 65, 67, 69, 71, 73, 75 and 76;
d. Operable fragments and variants of any of SEQ ID NO: 1, 61, 63, 65, 67, 69, 71, 73, 75 and 76;
e. A polynucleotide encoding a polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72 and 74;
f. A polynucleotide encoding a polypeptide at least 95% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72, and 74; and
g. A polynucleotide encoding a polypeptide at least 90% identical to any of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72, and 74.
